(19) United States
(12) Patent Application Publication
(10) Pub. No.: US 2011/0209253 A1
(43) Pub. Date: Aug. 25, 2011

(54) Method for Improving Plant Health

(75) Inventors: Klaus Grossmann, Neuhofen (DE); Petra Volz, Neustadt (DE); Matthias Witschel, Bad Duerkheim (DE)

(73) Assignee: BASF SE, Ludwigshafen (DE)

(21) Appl. No.: 13/126,673

(22) PCT Filed: Oct. 27, 2009
(86) PCT No.: PCT/EP09/64118

§ 371 Date: Apr. 28, 2011

(30) Foreign Application Priority Data
Oct. 31, 2008 (EP) 08168094.4

(51) Int. Cl. A01N 43/56; A01N 43/08; A01N 43/16; A01H 5/08; A01H 5/10; A01P 21/00

(52) U.S. Cl. 800/298; 504/280; 504/299; 504/292

(57) ABSTRACT

A method for improving the health of a plant, which method comprises treating the plant and/or the locus where the plant is growing or is intended to grow and/or the plant propagules with a plant health improving amount of at least one heteroaroyl-substituted alanine compound (A) of the formula I,

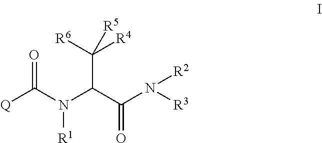

in which the variables are defined in the description.

The present invention relates to a method for improving the health of a plant, which method comprises treating the plant and/or the locus where the plant is growing or is intended to grow and/or the plant propagules with a plant health improving amount of at least one heteroaroyl-substituted alanine compound (A) of the formula I,

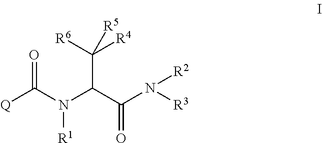

in which the variables are as defined below:
Q is 5- or 6-membered heteroaryl having one to four nitrogen atoms, or having one to three nitrogen atoms and one oxygen or sulfur atom, or having one oxygen or sulfur atom, wherein the heteroaryl may be partially or fully halogenated and/or may carry 1 to 3 radicals from the group consisting of hydroxyl, cyano, C₁-C₆-alkyl, C₃-C₆-cycloalkyl, C₁-C₆-haloalkyl, C₁-C₆-alkoxy, C₁-C₆-haloalkoxy or C₁-C₆-alkoxy-C₁-C₄-alkyl;
   R¹, R² are hydrogen or hydroxyl;
   R³ is C₁-C₆-alkyl, C₁-C₄-cyanoalkyl or C₁-C₆-haloalkyl;
   R⁴ is hydrogen, C₁-C₆-alkyl, haloalkyl or C₁-C₆-alkoxy-C₁-C₆-alkyl;
   R⁵ is hydrogen, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁-C₆-cyanoalkyl, C₂-C₆-cyanoalkenyl, C₂-C₆-cyanoalkynyl, C₁-C₆-hydroxyalkyl, C₂-C₆-hydroxyalkenyl, C₂-C₆-hydroxyalkynyl, C₃-C₆-cycloalkyl, C₃-C₆-cycloalkenyl, 3- to 6-membered heterocyclyl,
      wherein the cycloalkyl, cycloalkenyl or 3- to 6-membered heterocyclyl radicals mentioned above may be partially or fully halogenated and/or may carry one to three radicals from the group consisting of oxo, cyano, nitro, C₁-C₆-alkyl, C₁-C₆-haloalkyl, hydroxyl, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkoxycarbonyl-C₁-C₆-alkoxy, amino, C₁-C₆-alkylamino, di(C₁-C₆-alkyl)amino, C₁-C₆-alkylsulfonylamino, C₁-C₆-haloalkylsulfonylamino, aminocarbonylamino, (C₁-C₆-alkylamino)-carbonylamino, di(C₁-C₆-alkyl)aminocarbonylamino,
   C₁-C₆-alkoxy-C₁-C₄-alkyl, C₂-C₆-alkenyloxy-C₁-C₄-alkyl, C₂-C₆-alkynyloxy-C₁-C₄-alkyl, C₁-C₆-haloalkoxy-C₁-C₄-alkyl, C₂-C₆-haloalkenyloxy-C₁-C₄-alkyl, C₂-C₆-haloalkynyloxy-C₁-C₄-alkyl, C₁-C₆-alkoxy-C₁-C₄-alkoxy-C₁-C₄-alkyl, C₂-C₆-alkenylthio-C₁-C₄-alkyl, C₂-C₆-alkynylthio-C₁-C₄-alkyl, C₁-C₆-haloalkyl-C₁-C₄-thioalkyl, C₂-C₆-haloalkenyl-C₁-C₄-thioalkyl, C₂-C₆-haloalkynyl-C₁-C₄-thioalkyl, C₁-C₆-alkylsulfinyl-C₁-C₄-alkyl, C₁-C₆-haloalkylsulfinyl-C₁-C₄-alkyl, C₁-C₆-alkylsulfonyl-C₁-C₄-alkyl, C₁-C₆-haloalkylsulfonyl-C₁-C₄-alkyl, amino-C₁-C₄-alkyl, C₁-C₆-alkylamino-C₁-C₄-alkyl, di(C₁-C₆-alkyl)amino-C₁-C₄-alkyl, C₁-C₆-alkylsulfonylamino-C₁-C₄-alkyl, C₁-C₆-alkylsulfonyl(C₁-C₆-alkyl)amino-C₁-C₄-alkyl, C₁-C₆-alkylcarbonyl, hydroxycarbonyl, C₁-C₆-alkoxycarbonyl, aminocarbonyl, C₁-C₆-alkylaminocarbonyl, di(C₁-C₆-alkyl)aminocarbonyl, formylamino-C₁-C₄-alkyl, C₁-C₆-alkoxycarbonylamino-C₁-C₄-alkyl, C₁-C₆-alkylcarbonyl-C₁-C₆-alkyl, hydroxycarbonyl-C₁-C₄-alkyl, C₁-C₆-alkoxycarbonyl-C₁-C₄-alkyl, C₁-C₆-haloalkoxycarbonyl-C₁-C₄-alkyl, C₁-C₆-alkylcarbonyloxy-C₁-C₄-alkyl, aminocarbonyl-C₁-C₄-alkyl, C₁-C₆-alkylaminocarbonyl-C₁-C₄-alkyl, di(C₁-C₆-alkyl)aminocarbonyl-C₁-C₄-alkyl, C₁-C₆-alkylcarbonylamino-C₁-C₄-alkyl, C₁-C₆-alkylcarbonyl-(C₁-C₆-alkylamino)-C₁-C₄-alkyl, (C₁-C₆-alkyl)aminocarbonyloxy-C₁-C₄-alkyl, di(C₁-C₆-alkyl)aminocarbonyloxy-C₁-C₄-alkyl, [(C₁-C₆-alkyl)aminocarbonylamino]C₁-C₄-alkyl, [di(C₁-C₆-alkyl)aminocarbonylamino]C₁-C₄-alkyl;
   phenyl, phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl, phenyl-C₂-C₄-haloalkynyl, phenyl-C₁-C₄-hydroxyalkyl, phenyl-C₂-C₄-hydroxyalkenyl, phenyl-C₂-C₄-hydroxyalkynyl, phenylcarbonyl-C₁-C₄-alkyl, phenylcarbonyloxy-C₁-C₄-alkyl, phenyloxycarbonyl-C₁-C₄-alkyl, phenyloxy-C₁-C₄-alkyl, phenylthio-C₁-C₄-alkyl, phenylsulfinyl-C₁-C₄-alkyl, phenylsulfonyl-C₁-C₄-alkyl, heteroaryl, heteroaryl-C₁-C₄-alkyl, heteroaryl-C₂-C₄-alkenyl, heteroaryl-C₂-C₄-alkynyl, heteroaryl-C₁-C₄-haloalkyl, heteroaryl-C₂-C₄-haloalkenyl, heteroaryl-C₂-C₄-haloalkynyl, heteroaryl-C₁-C₄-hydroxyalkyl, heteroaryl-C₂-C₄-hydroxyalkenyl, heteroaryl-C₂-C₄-hydroxyalkynyl, heteroarylcarbonyl-C₁-C₄-alkyl, heteroarylcarbonyloxy-C₁-C₄-alkyl, heteroaryloxycarbonyl-C₁-C₄-alkyl, heteroaryloxy-C₁-C₄-alkyl, heteroarylthio-C₁-C₄-alkyl, heteroarylsulfinyl-C₁-C₄-alkyl, heteroarylsulfonyl-C₁-C₄-alkyl,
      wherein the phenyl and heteroaryl radicals mentioned above may be partially or fully halogenated and/or may carry one to three radicals from the group consisting of cyano, nitro, C₁-C₆-alkyl, C₁-C₆-haloalkyl, hydroxyl, C₁-C₆-hydroxyalkyl, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkoxycarbonyl-C₁-C₆-alkoxy, amino, C₁-C₆-alkylamino, di(C₁-C₆-alkyl)amino, C₁-C₆-alkylsulfonyl , C₁-C₆-alkylsulfinyl, C₁-C₆-alkylthio, C₁-C₆-alkylsulfonylamino, C₁-C₆-haloalkylsulfonylamino, (C₁-C₆-alkylamino)carbonylamino, di(C₁-C₆-alkyl)-aminocarbonylamino, aryl and aryl(C₁-C₆-alkyl); or
   R⁴ and R⁵ form together a 3-10-membered ring which may contain one to four nitrogen atoms, one to three nitrogen atoms and one oxygen or sulfur atom, one oxygen or sulfur atom, or 2 oxygen atoms whereas the ring may be partially or fully halogenated and/or may carry 1 to 3 radicals from the group consisting of cyano, C₁-C₆-alkyl, C₃-C₆-cycloalkyl, C₁-C₆-haloalkyl, C₁-C₆-alkoxy or C₁-C₆-haloalkoxy and C₁-C₆-alkoxy-C₁-C₄-alkyl;
   R⁶ is hydrogen, C₁-C₆-alkyl or OR⁷ or NR⁸R⁹
   R⁷ is hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, formyl, C₁-C₆-alkylcarbonyl, C₃-C₆-cycloalkylcarbonyl, C₂-C₆-alkenylcarbonyl, C₂-C₆-alkynylcarbonyl, C₁-C₆-alkoxycarbonyl, C₃-C₆-alkenyloxycarbonyl, C₃-C₆-alkynyloxycarbonyl, aminocarbonyl, C₁-C₆-alkylaminocarbonyl, C₃-C₆-alkenylaminocarbonyl, C₃-C₆-alkynylaminocarbonyl, C₁-C₆-alkylsulfonylaminocarbonyl, di(C₁-C₆-alkyl)aminocarbonyl, N—(C₃-C₆-alkenyl)-N—(C₁-C₆-alkyl)aminocarbonyl, N—(C₃-C₆-alkynyl)-N—(C₁-C₆-alkyl)aminocarbonyl, N—(C₁-C₆-alkoxy)-N—(C₁-C₆-alkyl)aminocarbonyl, N—(C₃-C₆-alkenyl)-N—(C₁-C₆-alkoxy)aminocarbonyl, N—(C₃-C₆-alkynyl)-N—(C₁-C₆-alkoxy)aminocarbonyl,C₁-C₆-alkylaminothiocarbonyl, di(C₁-C₆-alkyl)aminothiocarbonyl, (C₁-C₆-alkyl)cyanoimino, (amino)cyanoimino, [(C₁-C₆-alkyl)amino]cyanoimino, di(C₁-C₆-alkyl)aminocyanoimino, C₁-C₆-alkoxyimino-C₁-C₆-alkyl, N—(C₁-C₆-alkylamino)imino-C₁-C₆-alkyl, N-[di(C₁-C₆-alkyl)amino]imino-C₁-C₆-alkyl or tri-C₁-C₄-alkylsilyl,
      wherein the alkyl, cycloalkyl and alkoxy radicals mentioned above may be partially or fully halogenated and/or may carry one to three of the following groups: cyano, hydroxyl, C₃-C₆-cycloalkyl, C₁-C₆-alkoxy-C₁-C₄-alkyl, C₁-C₄-alkoxy-C₁-C₄-alkoxy-C₁-C₄-alkyl, C₁-C₄-alkoxy, C₁-C₄-alkylthio, di(C₁-C₄-alkyl)amino, C₁-C₄-alkyl-C₁-C₆-alkoxycarbonylamino, C₁-C₄-alkylcarbonyl, hydroxycarbonyl, C₁-C₄-alkoxycarbonyl, aminocarbonyl, C₁-C₄-alkylaminocarbonyl, di(C₁-C₄-alkyl)aminocarbonyl or C₁-C₄-alkylcarbonyloxy;
   phenyl, phenyl-C₁-C₆-alkyl, phenylcarbonyl-C₁-C₆-alkyl, phenoxycarbonyl, phenylaminocarbonyl, phenylsulfonylaminocarbonyl, N—(C₁-C₆-alkyl)-N-(phenyl)aminocarbonyl, phenyl-C₁-C₆-alkylcarbonyl, phenylcarbonyl,
      wherein the phenyl radical may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy;
   R⁸ is hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, formyl, C₁-C₆-alkylcarbonyl, C₃-C₆-cycloalkylcarbonyl, C₂-C₆-alkenylcarbonyl, C₂-C₆-alkynylcarbonyl, C₁-C₆-alkoxycarbonyl, C₃-C₆-alkenyloxycarbonyl, C₃-C₆-alkynyloxycarbonyl, aminocarbonyl, C₁-C₆-alkylaminocarbonyl, C₃-C₆-alkenylaminocarbonyl, C₃-C₆-alkynylaminocarbonyl, C₁-C₆-alkylsulfonylaminocarbonyl, di(C₁-C₆-alkyl)aminocarbonyl, N—(C₃-C₆-alkenyl)-N—(C₁-C₆-alkyl)aminocarbonyl, N—(C₃-C₆-alkynyl)-N—(C₁-C₆-alkyl)aminocarbonyl, N—(C₁-C₆-alkoxy)-N—(C₁-C₆-alkyl)aminocarbonyl, N—(C₃-C₆-alkenyl)-N—(C₁-C₆-alkoxy)aminocarbonyl, N—(C₃-C₆-alkynyl)-N—(C₁-C₆-alkoxy)aminocarbonyl, di(C₁-C₆-alkyl)aminothiocarbonyl, (C₁-C₆-alkyl)cyanoimino, (amino)cyanoimino, [(C₁-C₆-alkyl)amino]cyanoimino, di(C₁-C₆-alkyl)aminocyanoimino, C₁-C₆-alkylcarbonyl-C₁-C₆-alkyl, C₁-C₆-alkoxyimino-C₁-C₆-alkyl, N—(C₁-C₆-alkylamino)imino-C₁-C₆-alkyl, N-[di(C₁-C₆-alkyl)amino]imino-C₁-C₆-alkyl
      wherein, the alkyl, cycloalkyl and alkoxy radicals mentioned may be partially or fully halogenated and/or may carry one to three of the following groups: cyano, hydroxyl, C₃-C₆-cycloalkyl, C₁-C₆-alkoxy-C₁-C₄-alkyl, C₁-C₄-alkoxy-C₁-C₄-alkoxy-C₁-C₄-alkyl, C₁-C₄-alkoxy, C₁-C₄-alkylthio, di(C₁-C₄-alkyl)amino, C₁-C₄-alkyl-C₁-C₆-alkoxycarbonylamino, C₁-C₄-alkylcarbonyl, hydroxycarbonyl, C₁-C₄-alkoxycarbonyl, aminocarbonyl, C₁-C₄-alkylaminocarbonyl, di(C₁-C₄-alkyl)aminocarbonyl or C₁-C₄-alkylcarbonyloxy;
   phenyl, phenyl-C₁-C₆-alkyl, phenylcarbonyl-C₁-C₆-alkyl, phenoxycarbonyl, phenylaminocarbonyl, phenylsulfonylaminocarbonyl, N—(C₁-C₆-alkyl)-N-(phenyl)aminocarbonyl, phenyl-C₁-C₆-alkylcarbonyl, phenylcarbonyl wherein the phenyl radical may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy;
   R⁹ is hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, hydroxyl or C₁-C₆-alkoxy;

or an agriculturally useful salt thereof.

The present invention also relates to the use of at least one compound (A) of the formula I or an agriculturally useful salt thereof wherein the health of a plant and/or its propagules is improved.

The present invention is also directed to fruits produced from a plant treated with at least one compound (A) of the formula I or an agriculturally useful salt thereof.

The present invention is furthermore directed to seeds, treated with at least one compound (A) of the formula I or an agriculturally useful salt thereof.

In crop protection, there is a continuous need for compositions that improve the health of plants. Healthier plants are desirable since they result in higher yields and/or a better quality of a plant or its products. In addition, due to their increased vigor, healthier plants show a better resistance to biotic and/or abiotic stress.

A high resistance against biotic stress in turn allows the person skilled in the art to reduce the quantity of pesticides applied. Consequently, the potential development of resistances against the respective pesticides may be delayed or even prevented.

It was therefore an object of the present invention to provide a novel method which results in the positive effects, described above. In particular, the method should improve the health of plants (also referred to as plant health) resulting, among others, in an increased yield.

It has been found that this object is achieved by a method comprising treating the plant and/or the locus where the plant is growing or is intended to grow and/or the plant propagules with a plant health improving amount of a composition which comprises an effective and non-phytotoxic amount of at least one heteroaroyl-substituted alanine compound (A) of the formula I.

To date, heteroaroyl-substituted phenylalanine amides are known as herbicides from the literature, for example in WO 03/066576, WO 05/061464, WO 06/029829, WO 06/125687, WO 07/134984 and WO 07/093529.In JP 63 301 868 A an active ingredient is disclosed which encompasses a chlorinated pyridine substituted alanine derivative which is used for agricultural and horticultural fungicide compositions. Surprisingly it has been found that the action of compounds (A) of the formula I goes beyond the herbicidal and fungicidal activity. It could be shown that compounds (A) of the formula I exhibit plant health effects in the frame of the present invention.

Furthermore, it was surprisingly found that for achieving the above described object, only very low application rates of the composition comprising at least one heteroaroyl-substituted alanine compound (A) of the formula I are needed.

A reduction of the application rate not only displays environmental and economic advantages, it also leads to a better plant compatibility of the applied compositions.

Accordingly, the present invention relates to a method for improving the health of a plant, comprising treating the plant and/or the locus where the plant is growing or is intended to grow and/or the plant propagules with at least one compound (A) of the formula I or an agriculturally useful salt thereof.

In addition, the present invention relates to a method for increasing the yield of a plant, comprising treating the plant and/or the locus where the plant is growing or is intended to grow and/or the plant propagules with at least one compound (A) of the formula I or an agriculturally useful salt thereof.

Furthermore, the present invention relates to a method for increasing the vigor of a plant, comprising treating the plant and/or the locus where the plant is growing or is intended to grow and/or the plant propagules with at least one compound (A) of the formula I or an agriculturally useful salt thereof.

In addition, the present invention relates to a method for increasing the quality of a plant, comprising treating the plant and/or the locus where the plant is growing or is intended to grow and/or the plant propagules with at least one compound (A) of the formula I or an agriculturally useful salt thereof.

Moreover, the present invention relates to a method for increasing the stress tolerance of a plant, comprising treating the plant and/or the locus where the plant is growing or is intended to grow and/or the plant propagules with at least one compound (A) of the formula I or an agriculturally useful salt thereof.

In one embodiment the invention, the method comprises the application of a mixture comprising at least one compound (A) of the formula I or an agriculturally useful salt thereof, and at least one agriculturally active compound (B) or an agriculturally useful salt thereof.

Furthermore, the present invention relates to a method, which comprises applying in any desired sequence, simultaneously, that is, jointly or separately, or in succession, plant health effective amounts of a mixture comprising at least one compound (A) of the formula I or an agriculturally useful salt thereof, and at least one compound (B) or an agriculturally useful salt thereof.

In a preferred embodiment, the present invention relates to a method, which comprises applying in any desired sequence, simultaneously, that is, jointly or separately, or in succession, synergistically plant health effective amounts of a mixture comprising at least one compound (A) of the formula I or an agriculturally useful salt thereof, and at least one compound (B) or an agriculturally useful salt thereof.

This means that the purely additive (in mathematical terms) plant-health improving effect of at least one compound (A) of the formula I or an agriculturally useful salt thereof, and at least one compound (B) or an agriculturally useful salt thereof is surpassed by application of a mixture according to the invention. This synergistic effect is more than surprising, since normally it can be assumed that a herbicide and a further agriculturally active compound such a fungicide or insecticide have completely different mode of actions.

In addition, the present invention relates to a composition, comprising a plant health improving amount of at least one compound (A) of the formula I or an agriculturally useful salt thereof and auxiliaries customary for formulating crop protection agents.

In addition, the present invention is directed to the use of at least one compound (A) of the formula I or an agriculturally useful salt thereof for improving the health of a plant and/or its propagules.

The present invention is especially directed to the use of at least one compound (A) of the formula I or an agriculturally useful salt thereof for increasing the yield of a plant and/or its propagules.

The present invention is in particular directed to the use of at least one compound (A) of the formula I or an agriculturally useful salt thereof for increasing the stress tolerance of a plant and/or its propagules.

Furthermore, the present invention is directed to the use of at least one compound (A) of the formula I or an agriculturally useful salt thereof for increasing the yield and/or the vigor of a plant and/or its propagules.

In one embodiment of the invention, at least one compound (A) of the formula I or an agriculturally useful salt thereof is applied to plant propagules.

In another embodiment of the invention, at least one compound (A) of the formula I or an agriculturally useful salt thereof are used, wherein the yield of a plant and/or its propagules is increased.

In another embodiment of the invention, at least one compound (A) of the formula I or an agriculturally useful salt thereof are used, wherein the health of a plant and/or its propagules is improved.

In one embodiment of the invention, the present invention provides the use of the composition of the invention for increasing the yield of a plant or its product, preferably of an agricultural, silvicultural and/or horticultural plant. Thus, according to a preferred embodiment of the present invention, the inventive compositions are used for increasing the yield of a plant or its products.

In one embodiment of the invention, the composition of the invention is used for increasing the biomass and/or grain yield.

In one embodiment of the invention, the present invention provides the use of the composition of the invention for increasing the vigor of a plant or its products, preferably of an agricultural, silvicultural, and/or horticultural plant. Thus, according to a preferred embodiment of the present invention, the inventive compositions are used for increasing the vigor of a plant or its products.

In one embodiment of the invention, the composition of the invention is used for enhancing root and/or shoot growth.

In one embodiment of the invention, the present invention provides the use of the composition of the invention for increasing the quality of a plant or its products, preferably of an agricultural, silvicultural, and/or horticultural plant. Thus, according to a preferred embodiment of the present invention, the inventive compositions are used for increasing the quality of a plant or its products.

In one embodiment of the invention, the composition of the invention is used for improving metabolite composition and/or yield product quality, storability and/or processability.

In a preferred embodiment of the method according to the invention, the yield of a plant is increased.

In another preferred embodiment of the method according to the invention, the number of tillers is increased.

In yet another preferred embodiment of the method according to the invention, the shoot and/or root growth is increased

In one embodiment of the invention, the present invention provides the use of the composition of the invention for increasing the tolerance and/or resistance of a plant or its product against biotic and/or abiotic stress, preferably of an agricultural, silvicultural, and/or horticultural plant.

In one embodiment of the invention, the tolerance of and/or resistance against biotic stress factors is enhanced. Thus, according to a preferred embodiment of the present invention, the inventive compositions are used for stimulating the natural defensive reactions of a plant (plant strengthening effects) against biotic stress factors.

In one embodiment of the invention, the tolerance of and/or resistance against abiotic stress factors is enhanced. Thus, according to a further embodiment of the present invention, the inventive compositions are used for stimulating a plant's own defensive reactions against abiotic stress.

In one embodiment of the invention, the composition of the invention is used for stimulating a plant's own defensive reactions, enhancing their tolerance against biotic stress such as infections by fungi, bacteria, viruses, insects, arachnids and/or nematods.

In another embodiment of the invention, the inventive compositions are used for stimulating a plant's own defensive reactions against abiotic stress, where the abiotic stress factors are preferably selected from extremes in temperature, drought, salt and extreme wetness.

The compositions are used for improving the health of a plant when applied to a plant, parts of a plant, propagules of the plant or to its actual or intended locus of growth.

The organic moieties mentioned in the above definitions of the variables are—like the term halogen—collective terms for individual listings of the individual group members. The prefix Cₙ-Cₘ indicates in each case the possible number of carbon atoms in the group.

Unless indicated otherwise, halogenated substituents preferably carry one to five identical or different halogen atoms. The term halogen denotes in each case fluorine, chlorine, bromine or iodine.

The organic moieties mentioned for the substituents R¹-R⁹ or as radicals on phenyl, aryl, heteroaryl or heterocyclyl rings are collective terms for individual enumerations of the specific group members. All hydrocarbon chains, i.e. all alkyl, alkylsilyl, alkenyl, alkynyl, cyanoalkyl, haloalkyl, haloalkenyl, haloalkynyl, alkoxy, haloalkoxy, alkoxyalkyl, alkoxyalkoxyalkyl, alkylcarbonyl, alkenylcarbonyl, alkynylcarbonyl, alkoxycarbonyl, alkenyloxycarbonyl, alkynyloxycarbonyl, alkylamino, alkylsulfonylamino, haloalkylsulfonylamino, alkylalkoxycarbonylamino, alkylaminocarbonyl, alkenylaminocarbonyl, alkynylaminocarbonyl, alkylsulfonylaminocarbonyl, dialkylaminocarbonyl, N-alkenyl-N-alkylaminocarbonyl, N-alkynyl-N-alkylaminocarbonyl, N-alkoxy-N-alkylaminocarbonyl, N-alkenyl-N-alkoxyaminocarbonyl, N-alkynyl-N-alkoxyaminocarbonyl, dialkylaminothiocarbonyl, alkylcarbonylalkyl, alkoximinoalkyl, N-(alkylamino)iminoalkyl, N-(dialkylamino)iminoalkyl, formylamino-C₁-C₄-alkyl, C₁-C₆-alkoxycarbonylamino-C₁-C₄-alkyl, [(C₁-C₆-alkyl)aminocarbonylamino]C₁-C₄-alkyl, [di(C₁-C₆-alkyl)aminocarbonylamino]-C₁-C₄-alkyl, (C₁-C₆-alkyl)cyanoimino, [(C₁-C₆-alkyl)amino]cyanoimino, [di(C₁-C₆-alkyl)amino]cyanoimino, phenylalkyl, phenylcarbonylalkyl, N-alkyl-N-phenylaminocarbonyl, phenylalkylcarbonyl, arylalkyl, heterocyclylcarbonylalkyl, N-alkyl-N-heterocyclylaminocarbonyl, heterocyclylalkylcarbonyl, alkylthio and alkylcarbonyloxy moieties may be straight-chain or branched.

Examples of other meanings are:

C₁-C₄-alkyl and also the alkyl moieties of C₁-C₄-cyanoalkyl, tri-C₁-C₄-alkylsilyl, C₁-C₄-alkylcarbonyl, C₁-C₄-alkylcarbonyloxy, C₁-C₄-alkyl-C₁-C₆-alkoxycarbonylamino, C₁-C₄-alkoxy-C₁-C₄-alkoxy-C₁-C₄-alkyl, C₁-C₆-alkoxy-C₁-C₄-alkyl, C₂-C₆-alkenyloxy-C₁-C₄-alkyl, C₂-C₆-alkynyloxy-C₁-C₄-alkyl, C₂-C₆-haloalkenyloxy-C₁-C₄-alkyl, C₂-C₆-haloalkynyloxy-C₁-C₄-alkyl, alkoxy-C₁-C₄-alkyl, C₁-C₆-alkylthio-C₁-C₄-alkyl, C₂-C₆-alkenylthio-C₁-C₄-alkyl, C₂-C₆-alkynylthio-C₁-C₄-alkyl, C₁-C₆-alkylsulfinyl-C₁-C₄-alkyl, C₁-C₆-haloalkylsulfinyl-C₁-C₄-alkyl, C₁-C₆-alkylsulfonyl-C₁-C₄-alkyl, C₁-C₆-haloalkylsulfonyl-C₁-C₄-alkyl, alkyl, C₁-C₆-alkylamino-C₁-C₄-alkyl, di(C₁-C₆-alkyl)amino-C₁-C₄-alkyl, formylamino-C₁-C₄-alkyl, C₁-C₆-alkoxycarbonylamino-C₁-C₄-alkyl, C₁-C₆-alkylsulfonylamino-C₁-C₄-alkyl, C₁-C₆-alkylsulfonyl-(C₁-C₆-alkylamino)-C₁-C₄-alkyl, hydroxycarbonyl-C₁-C₄-alkyl, C₁-C₆-alkoxycarbonyl-C₁-C₄-alkyl, C₁-C₆-haloalkoxycarbonyl-C₁-C₄-alkyl, C₁-C₆-alkylcarbonyloxy-C₁-C₄-alkyl, aminocarbonyl-C₁-C₄-alkyl, C₁-C₆-alkylaminocarbonyl-C₁-di(C₁-C₆-alkyl)aminocarbonyl-C₁-C₄-alkyl, [(C₁-C₆-alkyl)aminocarbonylamino]C₁-C₄-alkyl, [di(C₁-C₆-alkyl)aminocarbonylamino]C₁-C₄-alkyl, C₁-C₆-alkylcarbonylamino-C₁-C₄-alkyl, C₁-C₆-alkylcarbonyl-(C₁-C₆-alkylamino)-C₁-C₄-alkyl, C₁-C₆-alkylaminocarbonyloxy-C₁-C₄-alkyl, [di(C₁-C₆-alkylamino)carbonyloxy]C₁-C₄-alkyl, phenyl-C₁-C₄-alkyl, phenylcarbonyl-C₁-C₄-alkyl, phenylcarbonyloxy-C₁-C₄-alkyl, phenyloxycarbonyl-C₁-C₄-alkyl, phenyloxy-C₁-C₄-alkyl, phenylthio-C₁-C₄-alkyl, phenylsulfinyl-C₁-C₄-alkyl, phenylsulfonyl-C₁-C₄-alkyl, heteroarylcarbonyl-C₁-C₄-alkyl, heteroarylcarbonyloxy-C₁-C₄-alkyl, heteroaryloxycarbonyl-C₁-C₄-alkyl, heteroaryloxy-C₁-C₄-alkyl, heteroarylthio-C₁-C₄-alkyl, heteroarylsulfinyl-C₁-C₄-alkyl and heteroarylsulfonyl-C₁-C₄-alkyl:

for example methyl, ethyl, n-propyl, 1-methylethyl, n-butyl, 1-methylpropyl, 2-methylpropyl and 1,1-dimethylethyl;

C₁-C₆-alkyl and also the alkyl moieties of C₁-C₆-cyanoalkyl, aryl(C₁-C₆-alkyl), phenyl-C₁-C₆-alkyl, phenylcarbonyl-C₁-C₆-alkyl, C₁-C₆-alkylsulfonylamino, C₁-C₆-alkylsulfonylaminocarbonyl, C₁-C₆-alkylcarbonyl-C₁-C₆-alkyl, alkyl, N-(di-C₁-C₆-alkylamino)imino-C₁-C₆-alkyl, (C₁-C₆-alkyl)cyanoimino and N—(C₁-C₆-alkyl)-N-phenylaminocarbonyl:

C₁-C₄-alkyl as mentioned above, and also, for example, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1-ethyl-1-methylpropyl and 1-ethyl-3-methylpropyl;

C₁-C₄-alkylcarbonyl: for example methylcarbonyl, ethylcarbonyl, propylcarbonyl, 1-methylethylcarbonyl, butylcarbonyl, 1-methylpropylcarbonyl, 2-methylpropylcarbonyl or 1,1-dimethylethylcarbonyl;

C₁-C₆-alkylcarbonyl and also the alkylcarbonyl radicals of C₁-C₆-alkylcarbonyl-C₁-C₆-alkyl, phenyl-C₁-C₆-alkylcarbonyl, C₁-C₆-alkylcarbonyl-(C₁-C₆-alkylamino)-C₁-C₄-alkyl:

C₁-C₄-alkylcarbonyl as mentioned above, and also, for example, pentylcarbonyl, 1-methylbutylcarbonyl, 2-methylbutylcarbonyl, 3-methylbutylcarbonyl, 2,2-dimethylpropylcarbonyl, 1-ethylpropylcarbonyl, hexylcarbonyl, 1,1-dimethylpropylcarbonyl, 1,2-dimethylpropylcarbonyl, 1-methylpentylcarbonyl, 2-methylpentylcarbonyl, 3-methylpentylcarbonyl, 4-methylpentylcarbonyl, 1,1-dimethylbutylcarbonyl, 1,2-dimethylbutylcarbonyl, 1,3-dimethylbutylcarbonyl, 2,2-dimethylbutylcarbonyl, 2,3-dimethylbutylcarbonyl, 3,3-dimethylbutylcarbonyl, 1-ethylbutylcarbonyl, 2-ethylbutylcarbonyl, 1,1,2-trimethylpropylcarbonyl, 1,2,2-trimethylpropylcarbonyl, 1-ethyl-1-methylpropylcarbonyl or 1-ethyl-2-methylpropylcarbonyl;

C₃-C₆-cycloalkyl and also the cycloalkyl moieties of C₃-C₆-cycloalkylcarbonyl: monocyclic saturated hydrocarbons having 3 to 6 ring members, such as cyclopropyl, cyclobutyl, cyclopentyl and cyclohexyl;

C₃-C₆-cycloalkenyl: for example 1-cyclopropenyl, 2-cyclopropenyl, 1-cyclobutenyl, 2-cyclobutenyl, 1-cyclopentenyl, 2-cyclopentenyl, 1,3-cyclopentadienyl, 1,4-cyclopentadienyl, 2,4-cyclopentadienyl, 1-cyclohexenyl, 2-cyclohexenyl, 3-cyclohexenyl, 1,3-cyclohexadienyl, 1,4-cyclohexadienyl, 2,5-cyclohexadienyl;

C₃-C₆-alkenyl and also the alkenyl moieties of C₃-C₆-alkenyloxycarbonyl, C₃-C₆-alkenylaminocarbonyl, N—(C₃-C₆-alkenyl)-N—(C₁-C₆-alkyl)aminocarbonyl and N—(C₃-C₆-alkenyl)-N—(C₁-C₆-alkoxy)aminocarbonyl: for example 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

C₂-C₆-alkenyl and also the alkenyl moieties of C₂-C₆-alkenylcarbonyl, C₂-C₆-alkenyloxy-C₁-C₄-alkyl, C₂-C₆-alkenylthio-C₁-C₄-alkyl, phenyl-C₂-C₄-alkenyl, heteroaryl-C₂-C₄-alkenyl: C₃-C₆-alkenyl as mentioned above, and also ethenyl;

C₃-C₆-alkynyl and also the alkynyl moieties of C₃-C₆-alkynyloxycarbonyl, C₃-C₆-alkynyl-aminocarbonyl, N—(C₃-C₆-alkynyl)-N—(C₁-C₆-alkyl)aminocarbonyl, N—(C₃-C₆-alkynyl)-N—(C₁-C₆-alkoxy)aminocarbonyl: for example 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

C₂-C₆-alkynyl and also the alkynyl moieties of C₂-C₆-alkynylcarbonyl, C₂-C₆-alkynyloxy-C₁-C₄-alkyl, C₂-C₆-alkynylthio-C₁-C₄-alkyl, phenyl-C₂-C₄-alkynyl, heteroaryl-C₂-C₄-alkynyl: C₃-C₆-alkynyl as mentioned above, and also ethynyl;

C₁-C₄-cyanoalkyl: for example cyanomethyl, 1-cyanoeth-1-yl, 2-cyanoeth-1-yl, 1-cyanoprop-1-yl, 2-cyanoprop-1-yl, 3-cyanoprop-1-yl, 1-cyanoprop-2-yl, 2-cyanoprop-2-yl, 1-cyanobut-1-yl, 2-cyanobut-1-yl, 3-cyanobut-1-yl, 4-cyanobut-1-yl, 1-cyanobut-2-yl, 2-cyanobut-2-yl, 1-cyanobut-3-yl, 2-cyanobut-3-yl, 1-cyano-2-methylprop-3-yl, 2-cyano-2-methylprop-3-yl, 3-cyano-2-methylprop-3-yl and 2-cyanomethylprop-2-yl;

C₁-C₄-hydroxyalkyl and also the C₁-C₄-hydroxyalkyl moieties of phenyl-C₁-C₄-hydroxyalkyl, heteroaryl-C₁-C₄-hydroxyalkyl: for example hydroxymethyl, 1-hydroxyeth-1-yl, 2-hydroxyeth-1-yl, 1-hydroxyprop-1-yl, 2-hydroxyprop-1-yl, 3-hydroxyprop-1-yl, 1-hydroxyprop-2-yl, 2-hydroxyprop-2-yl, 1-hydroxybut-1-yl, 2-hydroxybut-1-yl, 3-hydroxybut-1-yl, 4-hydroxybut-1-yl, 1-hydroxybut-2-yl, 2-hydroxybut-2-yl, 1-hydroxybut-3-yl, 2-hydroxybut-3-yl, 1-hydroxy-2-methylprop-3-yl, 2-hydroxy-2-methylprop-3-yl, 3-hydroxy-2-methylprop-3-yl and 2-hydroxymethylprop-2-yl, 1,2-dihydroxyethyl, 1,2-dihydroxyprop-3-yl, 2,3-dihydroxyprop-3-yl, 1,2-dihydroxyprop-2-yl, 1,2-dihydroxybut-4-yl, 2,3-dihydroxybut-4-yl, 3,4-dihydroxybut-4-yl, 1,2-dihydroxybut-2-yl, 1,2-dihydroxy-but-3-yl, 2,3-dihydroxybut-3-yl, 1,2-dihydroxy-2-methylprop-3-yl, 2,3-dihydroxy-2-methylprop-3-yl;

C₁-C₆-hydroxyalkyl: C₁-C₄-hydroxyalkyl as mentioned above and also, for example, 1-hydroxypent-5-yl, 2-hydroxypent-5-yl, 3-hydroxypent-5-yl, 4-hydroxypent-5-yl, 5-hydroxypent-5-yl, 1-hydroxypent-4-yl, 2-hydroxypent-4-yl, 3-hydroxypent-4-yl, 4-hydroxypent-4-yl, 1-hydroxypent-3-yl, 2-hydroxypent-3-yl, 3-hydroxypent-3-yl, 1-hydroxy-2-methylbut-3-yl, 2-hydroxy-2-methylbut-3-yl, 3-hydroxy-2-methylbut-3-yl, 1-hydroxy-2-methylbut-4-yl, 2-hydroxy-2-methylbut-4-yl, 3-hydroxy-2-methylbut-4-yl, 4-hydroxy-2-methylbut-4-yl, 1-hydroxy-3-methylbut-4-yl, 2-hydroxy-3-methylbut-4-yl, 3-hydroxy-3-methylbut-4-yl, 4-hydroxy-3-methylbut-4-yl, 1-hydroxyhex-6-yl, 2-hydroxyhex-6-yl, 3-hydroxyhex-6-yl, 4-hydroxyhex-6-yl, 5-hydroxyhex-6-yl, 6-hydroxyhex-6-yl, 1-hydroxy-2-methylpent-5-yl, 2-hydroxy-2-methylpent-5-yl, 3-hydroxy-2-methylpent-5-yl, 4-hydroxy-2-methylpent-5-yl, 5-hydroxy-2-methylpent-5-yl, 1-hydroxy-3-methylpent-5-yl, 2-hydroxy-3-methylpent-5-yl, 3-hydroxy-3-methylpent-5-yl, 4-hydroxy-3-methylpent-5-yl, 5-hydroxy-3-methylpent-5-yl, 1-hydroxy-4-methylpent-5-yl, 2-hydroxy-4-methylpent-5-yl, 3-hydroxy-4-methylpent-5-yl, 4-hydroxy-4-methylpent-5-yl, 5-hydroxy-4-methylpent-5-yl, 1-hydroxy-5-methylpent-5-yl, 2-hydroxy-5-methylpent-5-yl, 3-hydroxy-5-methylpent-5-yl, 4-hydroxy-5-methylpent-5-yl, 5-hydroxy-5-methylpent-5-yl, 1-hydroxy-2,3-dimethylbut-4-yl, 2-hydroxy-2,3-dimethylbut-4-yl, 3-hydroxy-2,3-dimethylbut-4-yl, 4-hydroxy-2,3-dimethylbut-4-yl, 1,2-dihydroxypent-5-yl, 2,3-dihydroxypent-5-yl, 3,4-dihydroxy-pent-5-yl, 4,5-dihydroxypent-5-yl, 1,2-dihydroxypent-4-yl, 2,3-dihydroxypent-4-yl, 3,4-dihydroxypent-4-yl, 4,5-dihydroxypent-4-yl, 1,2-dihydroxypent-3-yl, 2,3-dihydroxypent-3-yl, 1,2-dihydroxy-2-methylbut-3-yl, 2,3-dihydroxy-2-methylbut-3-yl, 3,4-dihydroxy-2-methylbut-3-yl, 2-hydroxy-2-hydroxymethylbut-3-yl, 1,2-dihydroxy-2-methylbut-4-yl, 2,3-dihydroxy-2-methylbut-4-yl, 3,4-dihydroxy-2-methylbut-4-yl, 1,2-dihydroxy-3-methylbut-4-yl, 2,3-dihydroxy-3-methylbut-4-yl, 3,4-dihydroxy-3-methylbut-4-yl, 3-hydroxy-3-hydroxymethylbut-4-yl, 1,2-dihydroxyhex-6-yl, 2,3-dihydroxyhex-6-yl, 3,4-dihydroxyhex-6-yl, 4,5-dihydroxyhex-6-yl, 5,6-dihydroxyhex-6-yl, 1,2-dihydroxy-2-methylpent-5-yl, 2,3-dihydroxy-2-methylpent-5-yl, 3,4-dihydroxy-2-methylpent-5-yl, 4,5-dihydroxy-2-methylpent-5-yl, 2-hydroxy-2-hydroxymethylpent-5-yl, 1,2-dihydroxy-3-methylpent-5-yl, 2,3-dihydroxy-3-methylpent-5-yl, 3,4-dihydroxy-3-methylpent-5-yl, 4,5-dihydroxy-3-methylpent-5-yl, 3-hydroxy-3-hydroxymethylpent-5-yl, 1,2-dihydroxy-4-methylpent-5-yl, 2,3-dihydroxy-4-methylpent-5-yl, 3,4-dihydroxy-4-methylpent-5-yl, 4,5-dihydroxy-4-methylpent-5-yl, 4-hydroxy-4-hydroxymethylpent-5-yl, 1,2-dihydroxy-5-methylpent-5-yl, 2,3-dihydroxy-5-methylpent-5-yl, 3,4-dihydroxy-5-methylpent-5-yl, 4,5-dihydroxy-5-methylpent-5-yl, 5-hydroxy-5-hydroxymethylpent-5-yl, 1,2-dihydroxy-2,3-dimethylbut-4-yl, 2,3-dihydroxy-2,3-dimethylbut-4-yl, 3,4-dihydroxy-2,3-dimethylbut-4-yl, 2-hydroxy-2-hydroxymethyl-3-methylbut-4-yl, 3-hydroxy-3-hydroxymethyl-2-methylbut-4-yl;

C₁-C₄-haloalkyl and also the haloalkyl moieties of phenyl-C₁-C₄-haloalkyl, heteroaryl-C₁-C₄-haloalkyl: a C₁-C₄-alkyl radical as mentioned above which is partially or fully substituted by fluorine, chlorine, bromine and/or iodine, i.e., for example, chloromethyl, dichloromethyl, trichloromethyl, fluoromethyl, difluoromethyl, trifluoromethyl, chlorofluoromethyl, dichlorofluoromethyl, chlorodifluoromethyl, bromomethyl, iodomethyl, 2-fluoroethyl, 2-chioroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichioropropyl, 2-bromopropyl, 3-bromopropyl, 3,3,3-trifluoropropyl, 3,3,3-trichioropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chiorobutyl, 4-bromobutyl, nonafluorobutyl, 1,1,2,2-tetrafluoroethyl and 1-trifluoromethyl-1,2,2,2,2-tetrafluoroethyl;

C₁-C₆-haloalkyl and also the haloalkyl moieties of C₁-C₆-haloalkylsulfonylamino, C₁-C₆-haloalkyl-C₁-C₄-thioalkyl: C₁-C₄-haloalkyl as mentioned above, and also, for example, 5-fluoropentyl, 5-chloropentyl, 5-bromopentyl, 5-iodopentyl, undecafluoropentyl, 6-fluorohexyl, 6-chlorohexyl, 6-bromohexyl, 6-iodohexyl and tridecafluorohexyl;

C₃-C₆-haloalkenyl: a C₃-C₆-alkenyl radical as mentioned above which is partially or fully substituted by fluorine, chlorine, bromine and/or iodine, for example 2-chloroprop-2-en-1-yl, 3-chloroprop-2-en-1-yl, 2,3-dichloroprop-2-en-1-yl, 3,3-dichloroprop-2-en-1-yl, 2,3,3-trichloro-2-en-1-yl, 2,3-dichlorobut-2-en-1-yl, 2-bromoprop-2-en-1-yl, 3-bromoprop-2-en-1-yl, 2,3-dibromoprop-2-en-1-yl, 3,3-dibromoprop-2-en-1-yl, 2,3,3-tribromo-2-en-1-yl or 2,3-dibromobut-2-en-1-yl;

C₂-C₆-haloalkenyl and also the C₂-C₆-haloalkenyl moieties of C₂-C₆-haloalkenyloxy-C₁-C₄-alkyl, C₂-C₆-haloalkenyl-C₁-C₄-thioalkyl, phenyl-C₂-C₄-haloalkenyl, heteroaryl-C₂-C₄-haloalkenyl: a C₂-C₆-alkenyl radical as mentioned above which is partially or fully substituted by fluorine, chlorine, bromine and/or iodine: for example 2-chlorovinyl, 2-chloroallyl, 3-chloroallyl, 2,3-dichloroallyl, 3,3-dichloroallyl, 2,3,3-trichloroallyl, 2,3-dichlorobut-2-enyl, 2-bromovinyl, 2-bromoallyl, 3-bromoallyl, 2,3-dibromoallyl, 3,3-di-bromoallyl, 2,3,3-tribromoallyl or 2,3-dibromobut-2-enyl;

C₂-C₆-cyanoalkenyl: for example 2-cyanovinyl, 2-cyanoallyl, 3-cyanoallyl, 2,3-dicyanoallyl, 3,3-dicyanoallyl, 2,3,3-tricyanoallyl, 2,3-dicyanobut-2-enyl;

C₂-C₆-hydroxyalkenyl and also the hydroxyl moieties of phenyl-C₂-C₄-hydroxyalkenyl: for example 2-hydroxyvinyl, 2-hydroxyallyl, 3-hydroxyallyl, 2,3-dihydroxyallyl, 3,3-dihydroxyallyl, 2,3,3-trihydroxyallyl, 2,3-di hydroxybut-2-enyl;

C₃-C₆-haloalkynyl: a C₃-C₆-alkynyl radical as mentioned above which is partially or fully substituted by fluorine, chlorine, bromine and/or iodine, for example 1,1-difluoroprop-2-yn-1-yl, 3-iodoprop-2-yn-1-yl, 4-fluorobut-2-yn-1-yl, 4-chlorobut-2-yn-1-yl, 1,1-difluorobut-2-yn-1-yl, 4-iodobut-3-yn-1-yl, 5-fluoropent-3-yn-1-yl, 5-iodopent-4-yn-1-yl, 6-fluorohex-4-yn-1-yl or 6-iodohex-5-yn-1-yl;

C₂-C₆-haloalkynyl and also the C₂-C₆-haloalkynyl moieties of C₂-C₆-haloalkynyloxy-C₁-C₄-alkyl, C₂-C₆-haloalkynyl-C₁-C₄-thioalkyl, phenyl-C₂-C₄-haloalkynyl, heteroaryl-C₂-C₄-haloalkynyl: a C₂-C₆-alkynyl radical as mentioned above which is partially or fully substituted by fluorine, chlorine, bromine and/or iodine, for example 1,1-difluoroprop-2-yn-1-yl, 3-iodoprop-2-yn-1-yl, 4-fluorobut-2-yn-1-yl, 4-chlorobut-2-yn-1-yl, 1,1-difluorobut-2-yn-1-yl, 4-iodobut-3-yn-1-yl, 5-fluoropent-3-yn-1-yl, 5-iodopent-4-yn-1-yl, 6-fluorohex-4-yn-1-yl or 6-iodohex-5-yn-1-yl;

C₂-C₆-cyanoalkynyl: for example 1,1-dicyanoprop-2-yn-1-yl, 3-cyanoprop-2-yn-1-yl, 4-cyanobut-2-yn-1-yl, 1,1-dicyanobut-2-yn-1-yl, 4-cyanobut-3-yn-1-yl, 5-cyanopent-3-yn-1-yl, 5-cyanopent-4-yn-1-yl, 6-cyanohex-4-yn-1-yl or 6-cyanohex-5-yn-1-yl;

C₂-C₆-hydroxyalkynyl and also the hydroxyl moieties of phenyl-C₂-C₄-hydroxyalkynyl: for example 1,1-dihydroxyprop-2-yn-1-yl, 3-hydroxyprop-2-yn-1-yl, 4-hydroxybut-2-yn-1-yl, 1,1-dihydroxybut-2-yn-1-yl, 4-hydroxybut-3-yn-1-yl, 5-hydroxypent-3-yn-1-yl, 5-hydroxypent-4-yn-1-yl, 6-hydroxyhex-4-yn-1-yl or 6-hydroxyhex-5-yn-1-yl;

C₁-C₆-alkylsulfinyl (C₁-C₆-alkyl-S(═O)—) and also the C₁-C₆-alkylsulfinyl moieties of C₁-C₆-alkylsulfinyl-C₁-C₄-alkyl: for example methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 2,2-dimethylpropylsulfinyl, 1-ethylpropylsulfinyl, 1,1-dimethylpropylsulfinyl, 1,2-dimethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl;

C₁-C₆-haloalkylsulfinyl and also the C₁-C₆-haloalkylsulfinyl moieties of C₁-C₆-haloalkylsulfinyl-C₁-C₄-alkyl: C₁-C₆-alkylsulfinyl radical as mentioned above which is partially or fully substituted by fluorine, chlorine, bromine and/or iodine, i.e. for example fluoromethylsulfinyl, difluoromethylsulfinyl, trifluoromethylsulfinyl, chlorodifluoromethylsulfinyl, bromodifluoromethylsulfinyl, 2-fluoroethylsulfinyl, 2-chloroethylsulfinyl, 2-bromoethylsulfinyl, 2-iodoethylsulfinyl, 2,2-difluoroethylsulfinyl, 2,2,2-trifluoroethylsulfinyl, 2,2,2-trichloroethylsulfinyl, 2-chloro-2-fluoroethylsulfinyl, 2-chloro-2,2-difluoroethylsulfinyl, 2,2-dichloro-2-fluoroethylsulfinyl, pentafluoroethylsulfinyl, 2-fluoropropylsulfinyl, 3-fluoropropylsulfinyl, 2-chloropropylsulfinyl, 3-chloropropylsulfinyl, 2-bromopropylsulfinyl, 3-bromopropylsulfinyl, 2,2-difluoropropylsulfinyl, 2,3-difluoropropylsulfinyl, 2,3-dichloropropylsulfinyl, 3,3,3-trifluoropropylsulfinyl, 3,3,3-trichloropropylsulfinyl, 2,2,3,3,3-pentafluoropropylsulfinyl, heptafluoropropylsulfinyl, 1-(fluoromethyl)-2-fluoroethylsulfinyl, 1-(chloromethyl)-2-chloroethylsulfinyl, 1-(bromomethyl)-2-bromoethylsulfinyl, 4-fluorobutylsulfinyl, 4-chlorobutylsulfinyl, 4-bromobutylsulfinyl, nonafluorobutylsulfinyl, 5-fluoropentylsulfinyl, 5-chloropentyl-sulfinyl, 5-bromopentylsulfinyl, 5-iodopentylsulfinyl, undecafluoropentylsulfinyl, 6-fluorohexylsulfinyl, 6-chlorohexylsulfinyl, 6-bromohexylsulfinyl, 6-iodohexylsulfinyl and tridecafluorohexylsulfinyl;

C₁-C₆-alkylsulfonyl (C₁-C₆-alkyl-S(O)₂—) and also the C₁-C₆-alkylsulfonyl moieties of C₁-C₆-alkylsulfonylamino and C₁-C₆-alkylsulfonyl-(C₁-C₆-alkylamino)-C₁-C₄-alkyl: for example methylsulfonyl, ethylsulfonyl, propylsulfonyl, 1-methylethylsulfonyl, butylsulfonyl, 1-methylpropylsulfonyl, 2-methylpropylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl;

C₁-C₆-haloalkylsulfonyl and also the C₁-0₅-haloalkylsulfonyl moieties of C₁-C₆-haloalkylsulfonylamino: a C₁-C₆-alkylsulfonyl radical as mentioned above which is partially or fully substituted by fluorine, chlorine, bromine and/or iodine, i.e. for example fluoromethylsulfonyl, difluoromethylsulfonyl, trifluoromethylsulfonyl, chlorodifluoromethylsulfonyl, bromodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl, pentafluoroethylsulfonyl, 2-fluoropropylsulfonyl, 3-fluoropropylsulfonyl, 2-chloropropylsulfonyl, 3-chloropropylsulfonyl, 2-bromopropylsulfonyl, 3-bromopropylsulfonyl, 2,2-difluoropropylsulfonyl, 2,3-difluoropropylsulfonyl, 2,3-dichloropropylsulfonyl, 3,3,3-trifluoropropylsulfonyl, 3,3,3-trichloropropylsulfonyl, 2,2,3,3,3-pentafluoropropylsulfonyl, heptafluoropropylsulfonyl, 1-(fluoromethyl)-2-fluoroethylsulfonyl, 1-(chloromethyl)-2-chloroethylsulfonyl, 1-(bromomethyl)-2-bromoethylsulfonyl, 4-fluorobutylsulfonyl, 4-chlorobutylsulfonyl, 4-bromobutylsulfonyl, nonafluorobutylsulfonyl, 5-fluoropentylsulfonyl, 5-chloropentylsulfonyl, 5-bromopentylsulfonyl, 5-iodopentylsulfonyl, 6-fluorohexylsulfonyl, 6-bromohexylsulfonyl, 6-iodohexylsulfonyl and tridecafluorohexylsulfonyl;

C₁-C₄-alkoxy: for example methoxy, ethoxy, propoxy, 1-methylethoxy, butoxy, 1-methylpropoxy, 2-methylpropoxy and 1,1-dimethylethoxy;

C₁-C₆-alkoxy and also the alkoxy moieties of C₁-C₆-alkoxy-C₁-C₆-alkyl, hydroxycarbonyl-C₁-C₆-alkoxy, C₁-C₆-alkoxycarbonyl-C₁-C₆-alkoxy, N—(C₁-C₆-alkoxy)-N—(C₁-C₆-alkyl)aminocarbonyl, N—(C₃-C₆-alkenyl)-N—(C₁-C₆-alkoxy)aminocarbonyl, N—(C₃-C₆-alkynyl)-N—(C₁-C₆-alkoxy)aminocarbonyl and C₁-C₆-alkoxyimino-C₁-C₆-alkyl: C₁-C₄-alkoxy as mentioned above, and also, for example, pentoxy, 1-methylbutoxy, 2-methylbutoxy, 3-methoxylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy;

C₁-C₄-haloalkoxy: a C₁-C₄-alkoxy radical as mentioned above which is partially or fully substituted by fluorine, chlorine, bromine and/or iodine, i.e., for example, fluoromethoxy, difluoromethoxy, trifluoromethoxy, chlorodifluoromethoxy, bromodifluoromethoxy, 2-fluoroethoxy, 2-chloroethoxy, 2-bromomethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2-chloropropoxy, 3-chloropropoxy, 2-bromopropoxy, 3-bromopropoxy, 2,2-difluoropropoxy, 2,3-difluoropropoxy, 2,3-dichloropropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy and nonafluorobutoxy;

C₁-C₆-haloalkoxy and also the C₁-C₆-haloalkoxy moieties of C₁-C₆-haloalkoxy-C₁-C₄-alkyl, C₁-C₆-haloalkoxycarbonyl-C₁-C₄-alkyl: C₁-C₄-haloalkoxy as mentioned above, and also, for example, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy and tridecafluorohexoxy;

C₁-C₆-alkoxy-C₁-C₄-alkyl and also the C₁-C₆-alkoxy-C₁-C₄-alkyl moieties of C₁-C₆-alkoxy-C₁-C₄-alkoxy-C₁-C₄-alkyl: C₁-C₄-alkyl which is substituted by C₁-C₆-alkoxy as mentioned above, i.e., for example, methoxymethyl, ethoxymethyl, propoxymethyl, (1-methylethoxy)methyl, butoxymethyl, (1-methylpropoxy)methyl, (2-methylpropoxy)methyl, (1,1-dimethylethoxy)methyl, 2-(methoxy)ethyl, 2-(ethoxy)ethyl, 2-(propoxy)ethyl, 2-(1-methylethoxy)ethyl, 2-(butoxy)ethyl, 2-(1-methylpropoxy)ethyl, 2-(2-methylpropoxy)ethyl, 2-(1,1-dimethylethoxy)ethyl, 2-(methoxy)propyl, 2-(ethoxy)propyl, 2-(propoxy)propyl, 2-(1-methylethoxy)propyl, 2-(butoxy)propyl, 2-(1-methylpropoxy)propyl, 2-(2-methylpropoxy)propyl, 2-(1,1-dimethylethoxy)propyl, 3-(methoxy)propyl, 3-(ethoxy)propyl, 3-(propoxy)propyl, 3-(1-methylethoxy)propyl, 3-(butoxy)propyl, 3-(1-methylpropoxy)propyl, 3-(2-methylpropoxy)propyl, 3-(1,1-dimethylethoxy)propyl, 2-(methoxy)butyl, 2-(ethoxy)butyl, 2-(propoxy)butyl, 2-(1-methylethoxy)butyl, 2-(butoxy)butyl, 2-(1-methylpropoxy)butyl, 2-(2-methylpropoxy)butyl, 2-(1,1-dimethylethoxy)butyl, 3-(methoxy)butyl, 3-(ethoxy)butyl, 3-(propoxy)butyl, 3-(1-methylethoxy)butyl, 3-(butoxy)butyl, 3-(1-methylpropoxy)butyl, 3-(2-methylpropoxy)butyl, 3-(1,1-dimethylethoxy)butyl, 4-(methoxy)butyl, 4-(ethoxy)butyl, 4-(propoxy)butyl, 4-(1-methylethoxy)butyl, 4-(butoxy)butyl, 4-(1-methylpropoxy)butyl, 4-(2-methylpropoxy)butyl and 4-(1,1-dimethylethoxy)butyl;

C₁-C₄-alkoxycarbonyl: for example methoxycarbonyl, ethoxycarbonyl, propoxycarbonyl, 1-methylethoxycarbonyl, butoxycarbonyl, 1-methylpropoxycarbonyl, 2-methylpropoxycarbonyl or 1,1-dimethylethoxycarbonyl;

C₁-C₆-alkoxycarbonyl and also the alkoxycarbonyl moieties of C₁-C₆-alkoxycarbonyl-C₁-C₆-alkoxy and C₁-C₆-alkoxycarbonylamino-C₁-C₄-alkyl: C₁-C₄-alkoxycarbonyl as mentioned above, and also, for example, pentoxycarbonyl, 1-methylbutoxycarbonyl, 2-methylbutoxycarbonyl, 3-methylbutoxycarbonyl, 2,2-dimethylpropoxycarbonyl, 1-ethylpropoxycarbonyl, hexoxycarbonyl, 1,1-dimethylpropoxycarbonyl, 1,2-dimethylpropoxycarbonyl, 1-methylpentoxycarbonyl, 2-methylpentoxycarbonyl, 3-methylpentoxycarbonyl, 4-methylpentoxycarbonyl, 1,1-dimethylbutoxycarbonyl, 1,2-dimethylbutoxycarbonyl, 1,3-dimethylbutoxycarbonyl, 2,2-dimethylbutoxycarbonyl, 2,3-dimethylbutoxycarbonyl, 3,3-dimethylbutoxycarbonyl, 1-ethylbutoxycarbonyl, 2-ethylbutoxycarbonyl, 1,1,2-trimethylpropoxycarbonyl, 1,2,2-trimethylpropoxycarbonyl, 1-ethyl-1-methylpropoxycarbonyl or 1-ethyl-2-methylpropoxycarbonyl;

C₁-C₄-alkylthio and also the C₁-C₄-alkylthio moieties of C₁-C₆-haloalkyl-C₁-C₄-thioalkyl, C₂-C₆-haloalkenyl-C₁-C₄-thioalkyl, C₂-C₆-haloalkynyl-C₁-C₄-thioalkyl: for example methylthio, ethylthio, propylthio, 1-methylethylthio, butylthio, 1-methylpropylthio, 2-methylpropylthio and 1,1-dimethylethylthio;

C₁-C₆-alkylthio and also the C₁-C₆-alkylthio moieties of C₁-C₆-alkylthio-C₁-C₄-alkyl: C₁-C₄-alkylthio as mentioned above, and also, for example, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutyl-thio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio;

C₁-C₆-alkylamino and also the C₁-C₆-alkylamino radicals of C₁-C₆-alkylamino-C₁-C₄-alkyl, C₁-C₆-alkylcarbonyl-(C₁-C₆-alkylamino)-C₁-C₄-alkyl, (C₁-C₆-alkylamino)-carbonylamino and [(C₁-C₆-alkyl)amino]cyanoimino: for example methylamino, ethylamino, propylamino, 1-methylethylamino, butylamino, 1-methylpropylamino, 2-methylpropylamino, 1,1-dimethylethylamino, pentylamino, 1-methylbutylamino, 2-methylbutylamino, 3-methylbutylamino, 2,2-dimethylpropylamino, 1-ethylpropylamino, hexylamino, 1,1-dimethylpropylamino, 1,2-dimethylpropylamino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 2,2-dimethylbutylamino, 2,3-dimethylbutylamino, 3,3-dimethylbutylamino, 1-ethylbutylamino, 2-ethylbutylamino, 1,1,2-trimethylpropylamino, 1,2,2-trimethylpropylamino, 1-ethyl-1-methylpropylamino or 1-ethyl-2-methylpropylamino;

di(C₁-C₄-alkyl)amino: for example N,N-dimethylamino, N,N-diethylamino, N,N-dipropylamino, N,N-di-(1-methylethyl)amino, N,N-dibutylamino, N,N-di-(1-methylpropyl)amino, N,N-di-(2-methylpropyl)amino, N,N-di-(1,1-dimethylethyl)amino, N-ethyl-N-methylamino, N-methyl-N-propylamino, N-methyl-N-(1-methylethyl)amino, N-butyl-N-methylamino, N-methyl-N-(1-methylpropyl)amino, N-methyl-N-(2-methylpropyl)amino, N-(1,1-dimethylethyl)-N-methylamino, N-ethyl-N-propylamino, N-ethyl-N-(1-methylethyl)amino, N-butyl-N-ethylamino, N-ethyl-N-(1-methylpropyl)amino, N-ethyl-N-(2-methylpropyl)amino, N-ethyl-N-(1,1-dimethylethyl)amino, N-(1-methylethyl)-N-propylamino, N-butyl-N-propylamino, N-(1-methylpropyl)-N-propylamino, N-(2-methylpropyl)-N-propylamino, N-(1,1-dimethylethyl)-N-propylamino, N-butyl-N-(1-methylethyl)amino, N-(1-methylethyl)-N-(1-methylpropyl)amino, N-(1-methylethyl)-N-(2-methylpropyl)amino, N-(1,1-dimethylethyl)-N-(1-methylethyl)amino, N-butyl-N-(1-methylpropyl)amino, N-butyl-N-(2-methylpropyl)amino, N-butyl-N-(1,1-dimethylethyl)amino, N-(1-methylpropyl)-N-(2-methylpropyl)amino, N-(1,1-dimethylethyl)-N-(1-methylpropyl)amino and N-(1,1-dimethylethyl)-N-(2-methylpropyl)amino;

di(C₁-C₆-alkyl)amino and also the dialkylamino radicals of di(C₁-C₆-alkyl)amino-C₁-C₄-alkyl, di(C₁-C₆-alkyl)aminocyanoimino and [di(C₁-C₆-alkylamino)carbonyloxy]-C₁-C₄-alkyl: di(C₁-C₄-alkyl)amino as mentioned above, and also, for example, N,N-dipentylamino, N,N-dihexylamino, N-methyl-N-pentylamino, N-ethyl-N-pentylamino, N-methyl-N-hexylamino and N-ethyl-N-hexylamino;

(C₁-C₄-alkylamino)carbonyl: for example methylaminocarbonyl, ethylaminocarbonyl, propylaminocarbonyl, 1-methylethylaminocarbonyl, butylaminocarbonyl, 1-methylpropylaminocarbonyl, 2-methylpropylaminocarbonyl or 1,1-dimethylethylaminocarbonyl;

di(C₁-C₄-alkyl)aminocarbonyl: for example N,N-dimethylaminocarbonyl, N,N-diethylaminocarbonyl, N,N-di-(1-methylethyl)aminocarbonyl, N,N-dipropylaminocarbonyl, N,N-dibutylaminocarbonyl, N,N-di-(1-methylpropyl)aminocarbonyl, N,N-di-(2-methylpropyl)aminocarbonyl, N,N-di-(1,1-dimethylethyl)aminocarbonyl, N-ethyl-N-methylaminocarbonyl, N-methyl-N-propylaminocarbonyl, N-methyl-N-(1-methylethyl)aminocarbonyl, N-butyl-N-methylaminocarbonyl, N-methyl-N-(1-methylpropyl)aminocarbonyl, N-methyl-N-(2-methylpropyl)aminocarbonyl, N-(1,1-dimethylethyl)-N-methylaminocarbonyl, N-ethyl-N-propylaminocarbonyl, N-ethyl-N-(1-methylethyl)aminocarbonyl, N-butyl-N-ethylaminocarbonyl, N-ethyl-N-(1-methylpropyl)aminocarbonyl, N-ethyl-N-(2-methylpropyl)aminocarbonyl, N-ethyl-N-(1,1-dimethylethyl)aminocarbonyl, N-(1-methylethyl)-N-propylaminocarbonyl, N-butyl-N-propylaminocarbonyl, N-(1-methylpropyl)-N-propylaminocarbonyl, N-(2-methylpropyl)-N-propylaminocarbonyl, N-(1,1-dimethylethyl)-N-propylaminocarbonyl, N-butyl-N-(1-methylethyl)aminocarbonyl, N-(1-methylethyl)-N-(1-methylpropyl)aminocarbonyl, N-(1-methylethyl)-N-(2-methylpropyl)aminocarbonyl, N-(1,1-dimethylethyl)-N-(1-methylethyl)aminocarbonyl, N-butyl-N-(1-methylpropyl)aminocarbonyl, N-butyl-N-(2-methylpropyl)aminocarbonyl, N-butyl-N-(1,1-dimethylethyl)aminocarbonyl, N-(1-methylpropyl)-N-(2-methylpropyl)aminocarbonyl, N-(1,1-dimethylethyl)-N-(1-methylpropyl)aminocarbonyl or N-(1,1-dimethylethyl)-N-(2-methylpropyl)aminocarbonyl;

(C₁-C₆-alkylamino)carbonyl and also the (C₁-C₆-alkylamino)carbonyl moieties of (C₁-C₆-alkylamino)carbonylamino, and [(C₁-C₆-alkyl)aminocarbonylamino]C₁-C₄-alkyl: (C₁-C₄-alkylamino)carbonyl as mentioned above, and also, for example, pentylaminocarbonyl, 1-methylbutylaminocarbonyl, 2-methylbutylaminocarbonyl, 3-methylbutylaminocarbonyl, 2,2-dimethylpropylaminocarbonyl, 1-ethylpropylaminocarbonyl, hexylaminocarbonyl, 1,1-dimethylpropylaminocarbonyl, 1,2-dimethylpropylaminocarbonyl, 1-methylpentylaminocarbonyl, 2-methylpentylaminocarbonyl, 3-methylpentylaminocarbonyl, 4-methylpentylaminocarbonyl, 1,1-dimethylbutylaminocarbonyl, 1,2-dimethylbutylaminocarbonyl, 1,3-dimethylbutylaminocarbonyl, 2,2-dimethylbutylaminocarbonyl, 2,3-dimethylbutylaminocarbonyl, 3,3-dimethylbutylaminocarbonyl, 1-ethylbutylaminocarbonyl, 2-ethylbutylaminocarbonyl, 1,1,2-trimethylpropylaminocarbonyl, 1,2,2-trimethylpropylaminocarbonyl, 1-ethyl-1-methylpropylaminocarbonyl or 1-ethyl-2-methylpropylaminocarbonyl;

di(C₁-C₆-alkyl)aminocarbonyl and also the di(C₁-C₆-alkyl)aminocarbonyl moieties of di(C₁-C₆-alkyl)aminocarbonylamino, and [di(C₁-C₆-alkyl)aminocarbonylamino]C₁-C₄-alkyl: di(C₁-C₄-alkyl)aminocarbonyl as mentioned above, and also, for example, N-methyl-N-pentylaminocarbonyl, N-methyl-N-(1-methylbutyl)aminocarbonyl, N-methyl-N-(2-methylbutyl)aminocarbonyl, N-methyl-N-(3-methylbutyl)aminocarbonyl, N-methyl-N-(2,2-dimethylpropyl)aminocarbonyl, N-methyl-N-(1-ethylpropyl)aminocarbonyl, N-methyl-N-hexylaminocarbonyl, N-methyl-N-(1,1-dimethylpropyl)aminocarbonyl, N-methyl-N-(1,2-dimethylpropyl)aminocarbonyl, N-methyl-N-(1-methylpentyl)aminocarbonyl, N-methyl-N-(2-methylpentyl)aminocarbonyl, N-methyl-N-(3-methylpentyl)aminocarbonyl, N-methyl-N-(4-methylpentyl)aminocarbonyl, N-methyl-N-(1,1-dimethylbutyl)aminocarbonyl, N-methyl-N-(1,2-dimethylbutyl)aminocarbonyl, N-methyl-N-(1,3-dimethylbutyl)aminocarbonyl, N-methyl-N-(2,2-dimethylbutyl)aminocarbonyl, N-methyl-N-(2,3-dimethylbutyl)aminocarbonyl, N-methyl-N-(3,3-dimethylbutyl)aminocarbonyl, N-methyl-N-(1-ethylbutyl)aminocarbonyl, N-methyl-N-(2-ethylbutyl)aminocarbonyl, N-methyl-N-(1,1,2-trimethylpropyl)aminocarbonyl, N-methyl-N-(1,2,2-trimethylpropyl)aminocarbonyl, N-methyl-N-(1-ethyl-1-methylpropyl)aminocarbonyl, N-methyl-N-(1-ethyl-2-methylpropyl)aminocarbonyl, N-ethyl-N-pentylaminocarbonyl, N-ethyl-N-(1-methylbutyl)aminocarbonyl, N-ethyl-N-(2-methylbutyl)aminocarbonyl, N-ethyl-N-(3-methylbutyl)aminocarbonyl, N-ethyl-N-(2,2-dimethylpropyl)aminocarbonyl, N-ethyl-N-(1-ethylpropyl)aminocarbonyl, N-ethyl-N-hexylaminocarbonyl, N-ethyl-N-(1,1-dimethylpropyl)aminocarbonyl, N-ethyl-N-(1,2-dimethylpropyl)aminocarbonyl, N-ethyl-N-(1-methylpentyl)aminocarbonyl, N-ethyl-N-(2-methylpentyl)aminocarbonyl, N-ethyl-N-(3-methylpentyl)aminocarbonyl, N-ethyl-N-(4-methylpentyl)aminocarbonyl, N-ethyl-N-(1,1-dimethylbutyl)aminocarbonyl, N-ethyl-N-(1,2-dimethylbutyl)aminocarbonyl, N-ethyl-N-(1,3-dimethylbutyl)aminocarbonyl, N-ethyl-N-(2,2-dimethylbutyl)aminocarbonyl, N-ethyl-N-(2,3-dimethylbutyl)aminocarbonyl, N-ethyl-N-(3,3-dimethylbutyl)aminocarbonyl, N-ethyl-N-(1-ethylbutyl)aminocarbonyl, N-ethyl-N-(2-ethylbutyl)aminocarbonyl, N-ethyl-N-(1,1,2-trimethylpropyl)aminocarbonyl, N-ethyl-N-(1,2,2-trimethylpropyl)aminocarbonyl, N-ethyl-N-(1-ethyl-1-methylpropyl)aminocarbonyl, N-ethyl-N-(1-ethyl-2-methylpropyl)aminocarbonyl, N-propyl-N-pentylaminocarbonyl, N-butyl-N-pentylaminocarbonyl, N,N-dipentylaminocarbonyl, N-propyl-N-hexylaminocarbonyl, N-butyl-N-hexylaminocarbonyl, N-pentyl-N-hexylaminocarbonyl or N,N-dihexylaminocarbonyl;

di(C₁-C₆-alkyl)aminothiocarbonyl: for example N,N-dimethylaminothiocarbonyl, N,N-diethylaminothiocarbonyl, N,N-di-(1-methylethyl)aminothiocarbonyl, N,N-dipropylaminothiocarbonyl, N,N-dibutylaminothiocarbonyl, N,N-di-(1-methyl-propyl)aminothiocarbonyl, N,N-di-(2-methylpropyl)aminothiocarbonyl, N,N-di-(1,1-dimethylethyl)aminothiocarbonyl, N-ethyl-N-methylaminothiocarbonyl, N-methyl-N-propylaminothiocarbonyl, N-methyl-N-(1-methylethyl)aminothiocarbonyl, N-butyl-N-methylaminothiocarbonyl, N-methyl-N-(1-methylpropyl)aminothiocarbonyl, N-methyl-N-(2-methylpropyl)aminothiocarbonyl, N-(1,1-dimethylethyl)-N-methylaminothiocarbonyl, N-ethyl-N-propylaminothiocarbonyl, N-ethyl-N-(1-methylethyl)-aminothiocarbonyl, N-butyl-N-ethylaminothiocarbonyl, N-ethyl-N-(1-methylpropyl)-aminothiocarbonyl, N-ethyl-N-(2-methylpropyl)aminothiocarbonyl, N-ethyl-N-(1,1-dimethylethyl)aminothiocarbonyl, N-(1-methylethyl)-N-propylaminothiocarbonyl, N-butyl-N-propylaminothiocarbonyl, N-(1-methylpropyl)-N-propylaminothiocarbonyl, N-(2-methylpropyl)-N-propylaminothiocarbonyl, N-(1,1-dimethylethyl)-N-propylaminothiocarbonyl, N-butyl-N-(1-methylethyl)aminothiocarbonyl, N-(1-methylethyl)-N-(1-methylpropyl)aminothiocarbonyl, N-(1-methylethyl)-N-(2-methylpropyl)aminothiocarbonyl, N-(1,1-dimethylethyl)-N-(1-methylethyl)aminothiocarbonyl, N-butyl-N-(1-methylpropyl)aminothiocarbonyl, N-butyl-N-(2-methyl-propyl)aminothiocarbonyl, N-butyl-N-(1,1-dimethylethypaminothiocarbonyl, N-(1-methylpropyl)-N-(2-methylpropyl)aminothiocarbonyl, N-(1,1-dimethylethyl)-N-(1-methylpropyl)aminothiocarbonyl, N-(1,1-dimethylethyl)-N-(2-methylpropyl)-aminothiocarbonyl, N-methyl-N-pentylaminothiocarbonyl, N-methyl-N-(1-methylbutyl)aminothiocarbonyl, N-methyl-N-(2-methylbutyl)aminothiocarbonyl, N-methyl-N-(3-methylbutyl)aminothiocarbonyl, N-methyl-N-(2,2-dimethylpropyl)aminothiocarbonyl, N-methyl-N-(1-ethylpropyl)aminothiocarbonyl, N-methyl-N-hexylaminothiocarbonyl, N-methyl-N-(1,1-dimethylpropyl)aminothiocarbonyl, N-methyl-N-(1,2-dimethylpropyl)aminothiocarbonyl, N-methyl-N-(1-methylpentyl)-aminothiocarbonyl, N-methyl-N-(2-methyl pentyl)aminothiocarbonyl, N-methyl-N-(3-methylpentyl)aminothiocarbonyl, N-methyl-N-(4-methylpentyl)aminothiocarbonyl, N-methyl-N-(1,1-dimethylbutyl)aminothiocarbonyl, N-methyl-N-(1,2-dimethylbutyl)aminothiocarbonyl, N-methyl-N-(1,3-dimethylbutyl)aminothiocarbonyl, N-methyl-N-(2,2-dimethylbutyl)aminothiocarbonyl, N-methyl-N-(2,3-dimethylbutyl)aminothiocarbonyl, N-methyl-N-(3,3-dimethylbutyl)aminothiocarbonyl, N-methyl-N-(1-ethylbutyl)aminothiocarbonyl, N-methyl-N-(2-ethylbutyl)-aminothiocarbonyl, N-methyl-N-ethyl-N-(1,1,2-trimethylpropyl)aminothiocarbonyl, N-methyl-N-(1,2,2-trimethylpropyl)aminothiocarbonyl, N-methyl-N-(1-ethyl-1-methylpropyl)aminothiocarbonyl, N-methyl-N-(1-ethyl-2-methylpropyl)aminothiocarbonyl, N-ethyl-N-pentylaminothiocarbonyl, N-ethyl-N-(1-methylbutyl)aminothiocarbonyl, N-ethyl-N-(2-methylbutyl)aminothiocarbonyl, N-ethyl-N-(3-methylbutyl)aminothiocarbonyl, N-ethyl-N-(2,2-dimethylpropyl)aminothiocarbonyl, N-ethyl-N-(1-ethylpropyl)aminothiocarbonyl, N-ethyl-N-hexylaminothiocarbonyl, N-ethyl-N-(1,1-dimethylpropyl)aminothiocarbonyl, N-ethyl-N-(1,2-dimethylpropyl)aminothiocarbonyl, N-ethyl-N-(1-methylpentyl)aminothiocarbonyl, N-ethyl-N-(2-methylpentyl)aminothiocarbonyl, N-ethyl-N-(3-methylpentyl)aminothiocarbonyl, N-ethyl-N-(4-methylpentyl)aminothiocarbonyl, N-ethyl-N-(1,1-dimethylbutyl)aminothiocarbonyl, N-ethyl-N-(1,2-dimethylbutyl)aminothiocarbonyl, N-ethyl-N-(1,3-dimethylbutyl)-aminothiocarbonyl, N-ethyl-N-(2,2-dimethylbutyl)aminothiocarbonyl, N-ethyl-N-(2,3-dimethylbutyl)aminothiocarbonyl, N-ethyl-N-(3,3-dimethylbutyl)aminothiocarbonyl, N-ethyl-N-(1-ethylbutyl)aminothiocarbonyl, N-ethyl-N-(2-ethylbutyl)aminothiocarbonyl, N-ethyl-N-(1,1,2-trimethylpropyl)aminothiocarbonyl, N-ethyl-N-(1,2,2-trimethylpropyl)aminothiocarbonyl, N-ethyl-N-(1-ethyl-1-methylpropyl)-aminothiocarbonyl, N-ethyl-N-(1-ethyl-2-methylpropyl)aminothiocarbonyl, N-propyl-N-pentylaminothiocarbonyl, N-butyl-N-pentylaminothiocarbonyl, N,N-dipentyl-aminothiocarbonyl, N-propyl-N-hexylaminothiocarbonyl, N-butyl-N-hexylaminothiocarbonyl, N-pentyl-N-hexylaminothiocarbonyl or N,N-dihexylaminothiocarbonyl;

three- to six-membered heterocyclyl: monocyclic saturated or partially unsaturated hydrocarbons having three to six ring members as mentioned above which, in addition to carbon atoms, may contain one to four nitrogen atoms or one to three nitrogen atoms and one oxygen or sulfur atom or one to three oxygen atoms or one to three sulfur atoms and which may be attached via a carbon atom or a nitrogen atom,

for example 2-oxiranyl, 2-oxetanyl, 3-oxetanyl, 2-aziridinyl, 3-thiethanyl, 1-azetidinyl, 2-azetidinyl,

for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 1,2,3,4-tetrazolidin-5-yl;

for example 1-pyrrolidinyl, 2-isothiazolidinyl, 2-isothiazolidinyl, 1-pyrazolidinyl, 3-oxazolidinyl, 3-thiazolidinyl, 1-imidazolidinyl, 1,2,4-triazolidin-1-yl, 1,2,4-oxadiazolidin-2-yl, 1,2,4-oxadiazolidin-4-yl, 1,2,4-thiadiazolidin-2-yl, 1,2,4-thiadiazolidin-4-yl, 1,2,3,4-tetrazolidin-1-yl,

for example 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 4,5-dihydropyrrol-2-yl, 4,5-dihydropyrrol-3-yl, 2,5-dihydropyrrol-2-yl, 2,5-dihydropyrrol-3-yl, 4,5-dihydroisoxazol-3-yl, 2,5-dihydroisoxazol-3-yl, 2,3-dihydroisoxazol-3-yl, 4,5-dihydroisoxazol-4-yl, 2,5-dihydroisoxazol-4-yl, 2,3-dihydroisoxazol-4-yl, 4,5-dihydroisoxazol-5-yl, 2,5-dihydroisoxazol-5-yl, 2,3-dihydroisoxazol-5-yl, 4,5-dihydroisothiazol-3-yl, 2,5-dihydroisothiazol-3-yl, 2,3-dihydroisothiazol-3-yl, 4,5-dihydroisothiazol-4-yl, 2,5-dihydroisothiazol-4-yl, 2,3-dihydroisothiazol-4-yl, 4,5-dihydroisothiazol-5-yl, 2,5-dihydroisothiazol-5-yl, 2,3-dihydroisothiazol-5-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-3-yl, 3,4-dihydropyrazol-4-yl, 3,4-dihydropyrazol-5-yl, 4,5-dihydropyrazol-3-yl, 4,5-dihydropyrazol-4-yl, 4,5-dihydropyrazol-5-yl, 2,3-dihydroimidazol-2-yl, 2,3-dihydroimidazol-3-yl, 2,3-dihydroimidazol-4-yl, 2,3-dihydroimidazol-5-yl, 4,5-dihydroimidazol-2-yl, 4,5-dihydroimidazol-4-yl, 4,5-dihydroimidazol-5-yl, 2,5-dihydroimidazol-2-yl, 2,5-dihydroimidazol-4-yl, 2,5-dihydroimidazol-5-yl, 2,3-dihydrooxazol-3-yl, 2,3-dihydrooxazol-4-yl, 2,3-dihydrooxazol-5-yl, 3,4-dihydrooxazol-3-yl, 3,4-dihydrooxazol-4-yl, 3,4-dihydrooxazol-5-yl, 2,3-dihydrothiazol-3-yl, 2,3-dihydrothiazol-4-yl, 2,3-dihydrothiazol-5-yl, 3,4-dihydrothiazol-3-yl, 3,4-dihydrothiazol-4-yl, 3,4-dihydrothiazol-5-yl, 3,4-dihydrothiazol-2-yl, 3,4-dihydrothiazol-3-yl, 3,4-dihydrothiazol-4-yl,

for example 4,5-dihydropyrrol-1-yl, 2,5-dihydropyrrol-1-yl, 4,5-dihydroisoxazol-2-yl, 2,3-dihydroisoxazol-1-yl, 4,5-dihydroisothiazol-1-yl, 2,3-dihydroisothiazol-1-yl, 2,3-dihydropyrazol-1-yl, 4,5-dihydropyrazol-1-yl, 3,4-dihydropyrazol-1-yl, 2,3-dihydroimidazol-1-yl, 4,5-dihydroimidazol-1-yl, 2,5-dihydroimidazol-1-yl, 2,3-dihydrooxazol2-yl, 3,4-dihydrooxazol-2-yl, 2,3-dihydrothiazol-2-yl, 3,4-dihydrothiazol-2-yl;

for example 2-piperidinyl, 3-piperidinyl, 4-piperidinyl, 1,3-dioxan-2-yl, 1, 3-dioxan-4-yl, 1,3-dioxan-5-yl, 1,4-dioxan-2-yl, 1,3-dithian-2-yl, 1,4-dithian-3-yl, 1,3-dithian-4-yl, 1-4,dithian-2-yl, 2-tetrahydropyranyl, 3-tetrahydropyranyl, 4-tetrahydropyranyl, 2-tetrahydrothiopyranyl, 3-tetrahydrothiopyranyl, 4-tetrahydrothiopyranyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl, 1,2,4-hexahydrotriazin-3-yl, tetrahydro-1,3-oxazin-2-yl, tetrahydro-1,3-oxazin-6-yl, 2-morpholinyl, 3-morpholinyl, 1,3,5-trioxan-2-yl;

for example 1-piperidinyl, 1-hexahydropyridazinyl, 1-hexahydropyrimidinyl, 1-piperazinyl, 1,3,5-hexahydrotriazin-1-yl, 1,2,4-hexahydrotriazin-1-yl, tetrahydro-1,3-oxazin-1-yl, 1-morpholinyl;

for example 2H-pyran-2-yl, 2H-pyran-3-yl, 2H-pyran-4-yl, 2H-pyran-5-yl, 2H-pyran-6-yl, 3,6-dihydro-2H-pyran-2-yl, 3,6-dihydro-2H-pyran-3-yl, 3,6-dihydro-2H-pyran-4-yl, 3,6-dihydro-2H-pyran-5-yl, 3,6-dihydro-2H-pyran-6-yl, 3,4-dihydro-2H-pyran-3-yl, 3,4-dihydro-2H-pyran-4-yl, 3,4-dihydro-2H-pyran-6-yl, 2H-thiopyran-2-yl, 2H-thiopyran-3-yl, 2H-thiopyran-4-yl, 2H-thiopyran-5-yl, 2H-thiopyran-6-yl, 5,6-dihydro-4H-1,3-oxazin-2-yl;

aryl and the aryl moiety of aryl-(C₁-C₄-alkyl): a monocyclic to tricyclic aromatic carbocycle having 6 to 14 ring members, such as, for example, phenyl, naphthyl and anthracenyl;

heteroaryl and also the heteroaryl radicals in heteroaryl-C₁-C₄-alkyl, heteroaryl-C₁-C₄-alkyl, heteroaryl-C₂-C₄-alkenyl, heteroaryl-C₂-C₄-alkynyl, heteroaryl-C₁-C₄-haloalkyl, heteroaryl-C₂-C₄-haloalkenyl, heteroaryl-C₂-C₄-haloalkynyl, heteroaryl-C₁-C₄-hydroxyalkyl, heteroaryl-C₂-C₄-hydroxyalkenyl, heteroaryl-C₂-C₄-hydroxyalkynyl, heteroarylcarbonyl-C₁-C₄-alkyl, heteroarylcarbonyloxy-C₁-C₄-alkyl, heteroaryloxycarbonyl-C₁-C₄-alkyl, heteroaryloxy-C₁-C₄-alkyl, heteroarylthio-C₁-C₄-alkyl, heteroarylsulfinyl-C₁-C₄-alkyl, heteroarylsulfonyl-C₁-C₄-alkyl:

mono- or bicyclic aromatic heteroaryl having 5 to 10 ring members which, in addition to carbon atoms, contains 1 to 4 nitrogen atoms, or 1 to 3 nitrogen atoms and an oxygen or sulfur atom, or an oxygen or a sulfur atom, for example monocycles, such as furyl (for example 2-furyl, 3-furyl), thienyl (for example 2-thienyl, 3-thienyl), pyrrolyl (for example pyrrol-2-yl, pyrrol-3-yl), pyrazolyl (for example pyrazol-3-yl, pyrazol-4-yl), isoxazolyl (for example isoxazol-3-yl, isoxazol-4-yl, isoxazol-5-yl), isothiazolyl (for example isothiazol-3-yl, isothiazol-4-yl, isothiazol-5-yl), imidazolyl (for example imidazol-2-yl, imidazol-4-yl), oxazolyl (for example oxazol-2-yl, oxazol-4-yl, oxazol-5-yl), thiazolyl (for example thiazol-2-yl, thiazol-4-yl, thiazol-5-yl), oxadiazolyl (for example 1,2,3-oxadiazol-4-yl, 1,2,3-oxadiazol-5-yl, 1,2,4-oxadiazol-3-yl, 1,2,4-oxadiazol-5-yl, 1,3,4-oxadiazol-2-yl), thiadiazolyl (for example 1,2,3-thiadiazol-4-yl, 1,2,3-thiadiazol-5-yl, 1,2,4-thiadiazol-3-yl, 1,3,4-thiadiazolyl-2-yl), triazolyl (for example 1,2,3-triazol-4-yl, tetrazol-5-yl, pyridyl (for example pyridin-2-yl, pyridin-3-yl, pyridin-4-yl), pyrazinyl (for example pyridazin-3-yl, pyridazin-4-yl), pyrimidinyl (for example pyrimidin-2-yl, pyrimidin-4-yl, pyrimidin-5-yl), pyrazin-2-yl, triazinyl (for example 1,3,5-triazin-2-yl, 1,2,4-triazin-3-yl, 1,2,4-triazin-5-yl, 1,2,4-triazin-6-yl), tetrazinyl (for example 1,2,4,5-tetrazin-3-yl); and also bicycles such as the benzo-fused derivatives of the abovementioned monocycles, for example quinolinyl, isoquinolinyl, indolyl, benzothienyl, benzofuranyl, benzoxazolyl, benzothiazolyl, benzisothiazolyl, benzimidazolyl, benzopyrazolyl, benzothiadiazolyl, benzotriazolyl;

5- or 6-membered heteroaryl having one to four nitrogen atoms, or having one to three nitrogen atoms and one oxygen or sulfur atom, or having one oxygen or sulfur atom: for example aromatic 5-membered heterocycles which are attached via a carbon atom and which, in addition to carbon atoms, may contain one to four nitrogen atoms, or one to three nitrogen atoms and one oxygen or sulfur atom, or having one oxygen or sulfur atom as ring members, for example, 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;

for example aromatic 6-membered heterocycles which are attached via a carbon atom and which, in addition to carbon atoms, may contain one to four, preferably one to three nitrogen atoms as ring members, for example, 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

The invention relates to a method for improving the health of plants, which comprises treating the plant, a part of the plant, the locus where the plant is growing or is expected to grow, and/or the propagules from which the plant grows with the composition according to the invention comprising at least one compound (A) of formula I or an agriculturally useful salt thereof. According to the inventive method, the plant, the locus where the plant is growing or is expected to grow, and/or the propagules from which the plant is grown may also be treated with a mixture.

In the terms of the present invention “a mixture” means a combination of at least two active ingredients. In the present case, a mixture comprises at least one compound (A) of the formula I or an agriculturally useful salt thereof and at least another compound (B) or an agriculturally useful salt thereof.

In the terms of the present invention “composition” is not restricted to a physical mixture containing at least one compound (A) of formula I or an agriculturally useful salt thereof and/or a mixture of at least one compound (A) and at least one compound (B) but refers to any preparation form of components (A) and/or (B) the use of which is time- and locus-related. In one embodiment of the invention “composition” refers to a physical mixture of at least one compound (A) and at least one compound (B). In another embodiment of the invention, “composition” refers to the compounds (A) and (B) formulated separately but applied to the same plant, propagule or locus in a temporal relationship, i.e. simultaneously or subsequently, the subsequent application having a time interval which allows a combined action of the two compounds. One example for a composition wherein compound (A) and compound (B) are formulated separately is a two-component-kit. Accordingly, in the terms of the present invention, “composition” can also refer to a two-component-kit comprising a first compound which contains a liquid or solid carrier and optionally at least one surface-active compound and/or at least one conventional auxiliary and a second compound which contains a liquid or solid carrier and optionally at least one surface-active compound and/or at least one conventional auxiliary. Suitable liquid and solid carriers, surface-active compounds and auxiliaries are described below.

The compounds (A) of formula I, formula I.a, formula I.b and formula I.c mentioned above as well as compounds (B) as listed below can also be employed in the form of their agriculturally useful salts.

The below remarks as to preferred embodiments of compounds (A) and (B), to their preferred use and methods of using them are to be understood either each on their own or preferably in combination with each other.

In a particular embodiment, the variables of the heteroaroyl-substituted alanines of the formula I have the following meanings which, both on their own and in combination with one another are particular embodiments of the compounds of the formula I:

Preference is given to the heteroaroyl-substituted alanine compounds (A) of the formula I in which the variables are as defined below:
Q is 5-or 6-membered heteroaryl having one to four nitrogen atoms, or having one to three nitrogen atoms and one oxygen or sulfur atom, or having one oxygen or sulfur atom, wherein the heteroaryl may carry 1 to 3 radicals from the group consisting of C₁-C₆-alkyl or C₁-C₆-haloalkyl;
R¹, R² are hydrogen or hydroxyl;
R³ is C₁-C₆-alkyl or C₁-C₄-cyanoalkyl;
R⁵ is hydrogen, C₁-C₆-alkyl, C₂-C₆-alkenyl, C₂-C₆-alkynyl, C₁-C₆-haloalkyl, C₃-C₆-cycloalkyl, 3- to 6-membered heterocyclyl,
   where the cycloalkyl, cycloalkenyl or 3- to 6-membered heterocyclyl radicals mentioned above may carry one to three radicals from the group consisting of C₁-C₆-alkyl, C₁-C₆-haloalkyl, hydroxyl, C₁-C₆-alkoxy, C₁-C₆-haloalkoxy, di(C₁-C₆-alkyl)amino,
C₂-C₆-alkenyloxy-C₁-C₄-alkyl, C₂-C₆-alkynyloxy-C₁-C₄-alkyl, C₁-C₆-haloalkoxy-C₁-C₄-alkyl, C₁-C₆-alkoxy-C₁-C₄-alkoxy-C₁-C₄-alkyl, C₁-C₆-alkylthio-C₁-C₄-alkyl, C₁-C₆-alkylsulfonyl-C₁-C₄-alkyl,
Phenyl, phenyl-C₁-C₄-alkyl, phenyl-C₁-C₄-hydroxyalkyl, phenyloxy-C₁-C₄-alkyl, heteroaryl,
where the phenyl and heteroaryl radicals mentioned above may be partially or fully halogenated and/or may carry one to three radicals from the group consisting of cyano, nitro, C₁-C₆-alkyl, C₁-C₆-haloalkyl, hydroxyl, C₁-C₆-hydroxy alkyl, C₁-C₆-alkoxy or C₁-C₆-haloalkoxy,
or
R⁴ and R⁵ form together a 3-10-membered ring which may contain one to four nitrogen atoms, one nitrogen atom and one oxygen or sulfur atom, one oxygen atom, or 2 oxygen atoms and/or may carry 1 to 3 radicals from the group consisting of C₁-C₆-alkyl, C₁-C₆-haloalkyl, C₁-C₆-alkoxy, C₁-C₆-haloalkoxy and C₁-C₆-alkoxy-C₁-C₄-alkyl;
R⁶ is hydrogen or OR⁷ or NR⁸
R⁷ is hydrogen, C₁-C₆-alkyl, C₃-C₆-alkenyl, formyl, C₁-C₆-alkylcarbonyl, C₃-C₆-cycloalkylcarbonyl, C₂-C₆-alkenylcarbonyl, C₂-C₆-alkynylcarbonyl, C₁-C₆-alkoxycarbonyl, C₃-C₆-alkenyloxycarbonyl, C₃-C₆-alkynyloxycarbonyl, aminocarbonyl, C₁-C₆-alkylaminocarbonyl, C₃-C₆-alkenylaminocarbonyl, C₃-C₆-alkynylaminocarbonyl, di(C₁-C₆-alkyl)aminocarbonyl, N—(C₃-C₆-alkenyl)-N—(C₁-C₆-alkyl)aminocarbonyl, N—(C₃-C₆-alkynyl)-N—(C₁-C₆-alkyl)aminocarbonyl, N—(C₁-C₆-alkoxy)-N—(C₁-C₆-alkyl)aminocarbonyl, C¹-C⁶-alkylaminothocarbonyl ,di(C₁-C₆-alkyl)aminothiocarbonyl,
   where the alkyl, cycloalkyl and alkoxy radicals mentioned may carry one to three of the following groups: C₁-C₄-alkoxy, C₁-C₄-alkylthio;
   phenoxycarbonyl, N—(C₁-C₆-alkyl)-N-(phenyl)aminocarbonyl, phenylcarbonyl;
R⁸ is hydrogen, C₁-C₆-alkyl, formyl, C₁-C₆-alkylcarbonyl, aminocarbonyl, C₁-C₆-alkylaminocarbonyl, di(C₁-C₆-alkyl)aminocarbonyl, phenyl, phenyl carbonyl where the phenyl radical may be partially or fully halogenated and/or may carry one to three of the following groups: nitro, cyano, C₁-C₄-haloalkyl, C₁-C₄-alkoxy or C₁-C₄-haloalkoxy;
R⁹ is hydrogen or C₁-C₆-alkyl

or an agriculturally useful salt thereof.

Special preference is given to the heteroaroyl-substituted alanine compounds (A) of the formula I in which the variables are as defined below:
Q is 5-membered heteroaryl having one to four nitrogen atoms, or having one to three nitrogen atoms and one oxygen or sulfur atom, or having one oxygen or sulfur atom, which heteroaryl may carry 1 to 3 radicals from the group consisting of C₁-C₆-alkyl or C₁-C₆-haloalkyl;
R¹, R² are hydrogen;
R³ is C₁-C₆-alkyl;
R⁵ is C₁-C₆-alkyl, C₂-C₆-alkenyl, C₂-C₆-alkynyl, C₁-C₆-haloalkyl, C₃-C₆-cycloalkyl, 3- to 6-membered heterocyclyl,
   where the cycloalkyl, cycloalkenyl or 3- to 6-membered heterocyclyl radicals mentioned above may carry one to three radicals from the group consisting of C₁-C₆-alkyl, C₁-C₆-alkoxy,C₁-C₆-alkoxy-C₁-C₄-alkyl, C₂-C₆-alkenyloxy-C₁-C₄-alkyl, phenyl, heteroaryl,
      where the phenyl and heteroaryl radicals mentioned above may be partially or fully halogenated and/or may carry one to three radicals from the group consisting of C₁-C₆-alkyl, C₁-C₆-haloalkyl,
or
R⁴ and R⁵ form together a 4 to 6-membered ring that can be carbocyclic or contains one to four nitrogen atoms, or contains one nitrogen atom and one oxygen or sulfur atom, or contains one oxygen atom, or contains 2 oxygen atoms and/or may carry 1 to 3 radicals from the group consisting of C₁-C₆-alkyl, C₁-C₆-alkoxy,
R⁶ is hydrogen or OR⁷ or NR⁸R⁹
R⁷ is hydrogen, formyl, C₁-C₆-alkylcarbonyl, aminocarbonyl, C₁-C₆-alkylamino carbonyl, di(C₁-C₆-alkyl)aminocarbonyl, N—(C₁-C₆-alkyl)-N-(phenyl)amino carbonyl;
R⁸ is formyl, C₁-C₆-alkylcarbonyl, aminocarbonyl, C₁-C₆-alkylaminocarbonyl, di(C₁-C₆-alkyl)aminocarbonyl, phenylcarbonyl
   where the phenyl radical may be partially or fully halogenated;

or an agriculturally useful salt thereof.

The preferences given in connection with compounds (A) of formula I also apply to compounds of formulae I.a, I.b and I.c as defined below.

Particular preference is given to the compounds of formula I.a, I.b and I.c compiled in the tables 1 to 21 below, wherein the definitions for the substituents R⁴ and R⁵ are selected from Y-1 to Y-37 in table Y below.

The groups mentioned in the tables for a substituent are furthermore, independently of the combination wherein they are mentioned, a particularly preferred embodiment of the substituent in question.

In one embodiment, preference is given to the compounds of the formula I.a which correspond to formula I where Q is 1-methyl-3-trifluoromethyl-1H-pyrazol-4-yl:

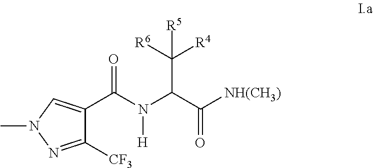

Table 1: Compounds of formula I.a, wherein R⁶ is H and the meaning of R⁴ and R⁵ for each individual compound corresponds in each case to one line of Table Y.

Table 2: Compounds of formula I.a, wherein R⁶ is OH and the meaning of R⁴ and R⁵ for each individual compound corresponds in each case to one line of Table Y.

Table 3: Compounds of formula I.a, wherein R⁶ is OCOCH₃ and the meaning of R⁴ and R⁵ for each individual compound corresponds in each case to one line of Table Y.

Table 4: Compounds of formula I.a, wherein R⁶ is OCON(CH₃)₂ and the meaning of R⁴ and R⁵ for each individual compound corresponds in each case to one line of Table Y.

Table 5: Compounds of formula I.a, wherein R⁶ is OCONHCH₃ and the meaning of R⁴ and R⁵ for each individual compound corresponds in each case to one line of Table Y.

Table 6: Compounds of formula I.a, wherein R⁶ is NHCON(CH₃)₂ and the meaning of R⁴ and R⁵ for each individual compound corresponds in each case to one line of Table Y.

Table 7: Compounds of formula I.a, wherein R⁶ is NHCO(o-F—C₆H₄) and the meaning of R⁴ and R⁵ for each individual compound corresponds in each case to one line of Table Y.

In one embodiment, preference is given to the compounds of the formula I.b which correspond to formula I where Q=5-fluoro-1-methyl-3-trifluoromethyl-1H-pyrazol-4-yl:

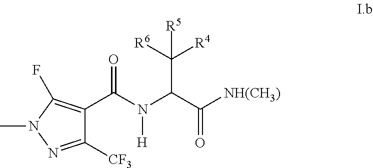

Table 8: Compounds of formula I.b, wherein R⁶ is H and the meaning of R⁴ and R⁵ for each individual compound corresponds in each case to one line of Table Y.

Table 9: Compounds of formula I.b, wherein R⁶ is OH and the meaning of R⁴ and R⁵ for each individual compound corresponds in each case to one line of Table Y.

Table 10: Compounds of formula I.b, wherein R⁶ is OCOCH₃ and the meaning of R⁴ and R⁵ for each individual compound corresponds in each case to one line of Table Y.

Table 11: Compounds of formula I.b, wherein R⁶ is OCON(CH₃)₂ and the meaning of R⁴ and R⁵ for each individual compound corresponds in each case to one line of Table Y.

Table 12: Compounds of formula I.b, wherein R⁶ is OCONHCH₃ and the meaning of R⁴ and R⁵ for each individual compound corresponds in each case to one line of Table Y.

Table 13: Compounds of formula I.b, wherein R⁶ is NHCON(CH₃)₂ and the meaning of R⁴ and R⁵ for each individual compound corresponds in each case to one line of Table Y.

Table 14: Compounds of formula I.b, wherein R⁶ is NHCO(o-F—C₆H₄) and the meaning of R⁴ and R⁵ for each individual compound corresponds in each case to one line of Table Y.

In one embodiment, preference is given to the compounds of the formula I.c which correspond to formula I where Q=5-methyl-2-trifluoromethyl-furan-3-yl:

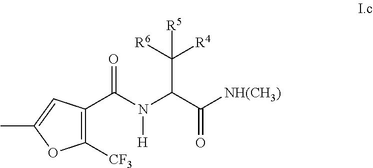

Table 15: Compounds of formula I.c, wherein R⁶ is H and the meaning of R⁴ and R⁵ for each individual compound corresponds in each case to one line of Table Y.

Table 16: Compounds of formula I.c, wherein R⁶ is OH and the meaning of R⁴ and R⁵ for each individual compound corresponds in each case to one line of Table Y.

Table 17: Compounds of formula I.c, wherein R⁶ is OCOCH₃ and the meaning of R⁴ and R⁵ for each individual compound corresponds in each case to one line of Table Y.

Table 18: Compounds of formula I.c, wherein R⁶ is OCON(CH₃)2 and the meaning of R⁴ and R⁵ for each individual compound corresponds in each case to one line of Table Y.

Table 19: Compounds of formula I.c, wherein R⁶ is OCONHCH₃ and the meaning of R⁴ and R⁵ for each individual compound corresponds in each case to one line of Table Y.

Table 20: Compounds of formula I.c, wherein R⁶ is NHCON(CH₃)₂ and the meaning of R⁴ and R⁵ for each individual compound corresponds in each case to one line of Table Y.

Table 21: Compounds of formula I.c, wherein R⁶ is NHCO(o-F—C₆H₄) and the meaning of R⁴ and R⁵ for each individual compound corresponds in each case to one line of Table Y.

TABLE Y

| No. | R⁴ | R⁵ |
|---|---|---|
| Y-1 | H | C₆H₄ |
| Y-2 | H | 2-Cl—C₆H₄ |
| Y-3 | H | 2-CH₃—C₆H₄ |
| Y-4 | H | 3-F-2-CH₃—C₆H₄ |
| Y-5 | H | CH₃ |
| Y-6 | H | C₂H₆ |
| Y-7 | H | CF₃ |
| Y-8 | H | CHCH₂ |
| Y-9 | H | CH₂OCH₃ |
| Y-10 | H | CH₂CH₂OCH₃ |
| Y-11 | H | 4-pyranyl |
| Y-12 | CH₃ | C₆H₄ |
| Y-13 | CH₃ | 2-Cl—C₆H₄ |
| Y-14 | CH₃ | 2-CH₃—C₆H₄ |
| Y-15 | CH₃ | 3-F-2-CH₃—C₆H₄ |
| Y-16 | CH₃ | CH₃ |
| Y-17 | CH₃ | C₂H₆ |
| Y-18 | CH₃ | CF₃ |
| Y-19 | CH₃ | CHCH₂ |
| Y-20 | CH₃ | CH₂OCH₃ |
| Y-21 | CH₃ | CH₂CH₂OCH₃ |
| Y-22 | CH₃ | 4-pyranyl |
| Y-23 | C₂H₆ | C₆H₄ |
| Y-24 | C₂H₆ | 2-Cl—C₆H₄ |
| Y-25 | C₂H₆ | 2-CH₃—C₆H₄ |
| Y-26 | C₂H₆ | 3-F-2-CH₃—C₆H₄ |
| Y-27 | C₂H₆ | CH₃ |
| Y-28 | C₂H₆ | C₂H₆ |
| Y-29 | C₂H₆ | CF₃ |
| Y-30 | C₂H₆ | CHCH₂ |
| Y-31 | C₂H₆ | CH₂OCH₃ |
| Y-32 | C₂H₆ | CH₂CH₂OCH₃ |
| Y-33 | C₂H₆ | 4-pyranyl |

| | |
|---|---|
| Y-34 | 3-oxetanyl |
| Y-35 | cyclobutyl |
| Y-36 | 2-CH₃-3-tetrahydrofuryl |
| Y-37 | 4-tetrahydropyranyl |

Especially preferred embodiments of the method according to the invention comprise the application of one compound (A) of formula I selected from the group of heteroaroyl-substituted alanines consisting of:

5-Methyl-2-trifluoromethyl-furan-3-carboxylic acid (1-methylcarbamoyl-2-phenyl-ethyl)-amide; 2-Methyl-5-trifluoromethyl-oxazole-4-carboxylic acid (1-methylcarbamoyl-2-phenyl-ethyl)-amide; 1-Methyl-3-trifluoromethyl-1H-pyrazole-4-carboxylic acid (1-methylcarbamoyl-2-phenyl-ethyl)-amide;

2-Trifluoromethyl-thiophene-3-carboxylic acid (1-methylcarbamoyl-2-phenyl-ethyl)-amide;

Methyl-carbamic acid 1-methyl-2-methylcarbamoyl-2-[(5-methyl-2-trifluoromethyl-furan-3-carbonyl)-amino]-ethyl ester;

Methyl-carbamic acid 1-methyl-2-methylcarbamoyl-2-[(1-methyl-3-trifluoromethyl-1H-pyrazole-4-carbonyl)-amino]-ethyl ester;

Methyl-carbamic acid 2-methylcarbamoyl-2-[(1-methyl-3-trifluoromethyl-1H-pyrazole-4-carbonyl)-amino]-1-(tetrahydro-pyran-4-yl)-ethyl ester;

Dimethyl-carbamic acid 2-methylcarbamoyl-2-[(1-methyl-3-trifluoromethyl-1H-pyrazole-4-carbonyl)-amino]-1-(tetrahydro-pyran-4-yl)-ethyl ester;

Methyl-carbamic acid 1,1-dimethyl-2-methylcarbamoyl-2-[(1-methyl-3-trifluoromethyl-1H-pyrazole-4-carbonyl)-amino]-ethyl ester;

Methyl-carbamic acid 1,1-dimethyl-2-methylcarbamoyl-2-[(5-methyl-2-trifluoromethyl-furan-3-carbonyl)-amino]-ethyl ester.

Another especially preferred embodiment of the method according to the invention comprises the application of one compound (A) of formula I selected from the group of heteroaroyl-substituted alanines consisting of:

Dimethyl-carbamic acid 2-methylcarbamoyl-2-[(1-methyl-3-trifluoromethyl-1H-pyrazole-4-carbonyl)-amino]-1-(tetrahydro-pyran-4-yl)-ethyl ester (compound 1);

5-Methyl-2-trifluoromethyl-furan-3-carboxylic acid (1-methylcarbamoyl-2-phenyl-ethyl)-amide (compound 2);

1-Methyl-3-trifluoromethyl-1H-pyrazole-4-carboxylic acid (1-methylcarbamoyl-2-phenyl-ethyl)-amide (compound 3);

Methyl-carbamic acid-2-methylcarbamoyl-2-[(1-methyl-3-trifluoromethyl-1H-pyrazole-4-carbonyl)-amino]-ethyl ester (compound 4);

Methyl-carbamic acid 2-methylcarbamoyl-2-[(1-methyl-3-trifluoromethyl-1H-pyrazole-4-carbonyl)-amino]-1-(tetrahydro-pyran-4-yl)-ethyl ester (compound 5);

Methyl-carbamic acid 1,1-dimethyl-2-methylcarbamoyl-2-[(1-methyl-3-trifluoromethyl-1H-pyrazole-4-carbonyl)-amino]-ethyl ester (compound 6);

1-Methyl-3-trifluoromethyl-1H-pyrazole-4-carboxylic acid (2-hydroxy-3-methoxy-2-methoxymethyl-1-methylcarbamoyl-propyl)-amide (compound 7);

Methyl-carbamic acid 1,1-bis-methoxymethyl-2-methylcarbamoyl-2-[(1-methyl-3-trifluoromethyl-1H-pyrazole-4-carbonyl)-amino]-ethyl ester (compound 8);

Methyl-carbamic acid (E)-1-methyl-1-{methylcarbamoyl-[(1-methyl-3-trifluoromethyl-1H-pyrazole-4-carbonyl)-amino]-methyl}-but-2-enyl ester (compound 9);

2-Methyl-5-trifluoromethyl-oxazole-4-carboxylic acid (1-methylcarbamoyl-2-phenylethyl)-amide;

2-Trifluoromethyl-thiophene-3-carboxylic acid (1-methylcarbamoyl-2-phenyl-ethyl)-amide;

Methyl-carbamic acid 1-methyl-2-methylcarbamoyl-2-[(5-methyl-2-trifluoromethyl-furan-3-carbonyl)-amino]-ethyl ester;

Methyl-carbamic acid 1-methyl-2-methylcarbamoyl-2-[(1-methyl-3-trifluoromethyl-1H-pyrazole-4-carbonyl)-amino]-ethyl ester; and

Methyl-carbamic acid 1,1-dimethyl-2-methylcarbamoyl-2-[(5-methyl-2-trifluoromethyl-furan-3-carbonyl)-amino]-ethyl ester.

In one embodiment according to the present invention, compound (A) is applied alone.

In the method according to the invention the application of one compound (A) of formula I usually is sufficient, however, it may be advantageously to use more than one compound (A) of formula I.

In one embodiment of the invention, it may also be an advantage to apply mixtures of at least one compound (A) of formula I and at least one active compound (B) as listed below to the plant and/or the locus where the plant is growing or is intended to grow and/or the plant propagules. Mixing the compounds (A) or the compositions comprising them with a further compound (A) and/or at least one further compound (B) results in many cases in an expansion of the plant health effect(s) being obtained.

Accordingly, the compound(s) (B) can be used as a synergist according to the invention for different active compounds (A) of formula I. When applied simultaneously that is by a joint or separate application of at least one compound (A) with at least one compound (B), the health of plants may be increased in a synergistic manner.

When preparing the mixtures, preference is given to using the pure active compounds (A) which, if required, may be mixed with further active compounds (B) for improving the health of a plant.

The following list of active compounds (B), in conjunction with which the compounds (A) according to the invention can be used, is intended to illustrate the possible combinations but does not limit them. Compounds (B), independently of the groups wherein they are mentioned, according to the invention are those selected from:

B.1) Strobilurins

azoxystrobin, dimoxystrobin, enestroburin, fluoxastrobin, kresoxim-methyl, metominostrobin, orysastrobin, picoxystrobin, pyraclostrobin, pyribencarb, trifloxystrobin, 2-(2-(6-(3-chloro-2-methyl-phenoxy)-5-fluoro-pyrimidin-4-yloxy)-phenyl)-2-methoxyimino-N-methyl-acetamide, 3-methoxy-2-(2-(N-(4-methoxy-phenyl)-cyclopropane-carboximidoylsulfanylmethyl)-phenylyacrylic acid methyl ester, methyl(2-chloro-5-[1-(3-methylbenzyloxyimino)ethyl]benzyl)carbamate and 2-(2-(3-(2,6-dichlorophenyl)-1-methyl-allylideneaminooxymethyl)-phenyl)-2-methoxyimino-N-methyl-acetamide;

B.2) Carboxamides

B.2.1) carboxanilides: benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M (mefenoxam), ofurace, oxadixyl, oxycarboxin, penthiopyrad, tecloftalam, thifluzamide, tiadinil, 2-amino-4-methyl-thiazole-5-carboxanilide, 2-chloro-N-(1,1,3-trimethyl-indan-4-yl)-nicotinamide, N-(2′,4′-difluorobiphenyl-2-yl)-3-difluoromethyl-1-methyl-1H-pyrazole-4-carboxamide, N-(2′,4′-dichlorobiphenyl-2-yl)-3-difluoromethyl-1-methyl-1H-pyrazole-4-carboxamide, N-(2′,5′-difluorobiphenyl-2-yl)-3-difluoromethyl-1-methyl-1H-pyrazole-4-carboxamide, N-(2′,5′-dichlorobiphenyl-2-yl)-3-difluoromethyl-1-methyl-1H-pyrazole-4-carboxamide, N-(3′,5′-difluorobiphenyl-2-yl)-3-difluoromethyl-1-methyl-1H-pyrazole-4-carboxamide, N-(3′-fluorobiphenyl-2-yl)-3-difluoromethyl-1-methyl-1H-pyrazole-4-carboxamide, N-(3′-chlorobiphenyl-2-yl)-3-difluoromethyl-1-methyl-1H-pyrazole-4-carboxamide, N-(2′-fluorobiphenyl-2-yl)-3-difluoromethyl-1-methyl-1H-pyrazole-4-carboxamide, N-(2′-chlorobiphenyl-2-yl)-3-difluoromethyl-1-methyl-1H-pyrazole-4-carboxamide, N-(3′,5′-dichlorobiphenyl-2-yl)-3-difluoromethyl-1-methyl-1H-pyrazole-4-carboxamide, N-(3′,4′,5′-trifluorobiphenyl-2-yl)-3-difluoromethyl-1-methyl-1H-pyrazole-4-carboxamide, N-(2′,4′,5′-trifluorobiphenyl-2-yl)-3-difluoromethyl-1-methyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[2-(1,1,2,2-tetrafluoroethoxy)-phenyl]-3-difluoromethyl-1-methyl-1H-pyrazole-4-carboxamide, N-(4′-trifluoromethylthiobiphenyl-2-yl)-3-difluoromethyl-1-methyl-1H-pyrazole-4-carboxamide, N-(2-(1,3-dimethyl-butyl)-phenyl)-1,3-dimethyl-5-fluoro-1H-pyrazole-4-carboxamide, N-(2-(1,3,3-trimethyl-butyl)-phenyl)-1,3-dimethyl-5-fluoro-1H-pyrazole-4-carboxamide, N-(4′-chloro-3′,5′-difluoro-biphenyl-2-yl)-3-difluoromethyl-1-methyl-1H-pyrazole-4-carboxamide, N-(4′-chloro-3′,5′-difluoro-biphenyl-2-yl)-3-trifluoromethyl-1-methyl-1H-pyrazole-4-carboxamide, N-(3′,4′-dichloro-5′-fluoro-biphenyl-2-yl)-3-trifluoromethyl-1-methyl-1H-pyrazole-4-carboxamide, N-(3′,5′-difluoro-4′-methyl-biphenyl-2-yl)-3-difluoromethyl-1-methyl-1H-pyrazole-4-carboxamide, N-(3′,5′-difluoro-4′-methyl-biphenyl-2-yl)-3-trifluoromethyl-1-methyl-1H-pyrazole-4-carboxamide, N-(2-bicyclopropyl-2-yl-phenyl)-3-difluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoromethyl-1-methyl-1H-pyrazole-4-carboxamide, N-[1,2,3,4-tetrahydro-9-(1-methylethyl)-1,4-methano-aphthalen-5-yl]-3-(difluoromethyl)-1-methyl-1 H-pyrazole-4-carboxamide;

B.2.2) carboxylic morpholides: dimethomorph, flumorph;

B.2.3) benzoic acid amides: flumetover, fluopicolde, fluopyram, zoxamide, N-(3-Ethyl-3,5,5-trimethyl-cyclohexyl)-3-formylamino-2-hydroxy-benzamide;

B.2.4) other carboxamides: carpropamid, dicyclomet, mandiproamid, oxytetracyclin, silthiofarm and N-(6-methoxy-pyridin-3-yl) cyclopropanecarboxylic acid amide;

B.3) Azoles

B.3.1) triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, 1-(4-chloro-phenyl)-2-([1,2,4]triazol-1-yl)-cycloheptanol;

B.3.2) imidazoles: cyazofamid, imazalil, pefurazoate, prochloraz, triflumizol;

B.3.3) benzimidazoles: benomyl, carbendazim, fuberidazole, thiabendazole;

B.3.4) others: ethaboxam, etridiazole, hymexazole and 2-(4-chloro-phenyl)-N-[4-(3,4-dimethoxy-phenyl)-isoxazol-5-yl]-2-prop-2-ynyloxy-acetamide;

B.4) Heterocyclic compounds

B.4.1) pyridines: fluazinam, pyrifenox, 3-[5-(4-chloro-phenyl)-2,3-dimethyl-isoxazolidin-3-yl]-pyridine, 3-[5-(4-methyl-phenyl)-2,3-dimethyl-isoxazolidin-3-yl]-pyridine, 2,3,5,6-tetra-chloro-4-methanesulfonyl-pyridine, 3,4,5-trichloropyridine-2,6-di-carbonitrile, N-(1-(5-bromo-3-chloro-pyridin-2-yl)-ethyl)-2,4-dichloronicotinamide, N-[(5-bromo-3-chloro-pyridin-2-yl)-methyl]-2,4-dichloro-nicotinamide;

B.4.2) pyrimidines: bupirimate, cyprodinil, diflumetorim, fenarimol, ferimzone, mepanipyrim, nitrapyrin, nuarimol, pyrimethanil;

B.4.3) piperazines: triforine;

B.4.4) morpholines: aldimorph, dodemorph, dodemorph-acetate, fenpropimorph, tridemorph;

B.4.5) piperidines: fenpropidin;

B.4.6) dicarboximides: fluoroimid, iprodione, procymidone, vinclozolin;

B.4.7) non-aromatic 5-membered heterocycles: famoxadone, fenamidone, flutianil, octhilinone, probenazole, 5-amino-2-isopropyl-3-oxo-4-ortho-tolyl-2,3-dihydro-pyrazole-1-carbothioic acid S-allyl ester;

B.4.8) others: acibenzolar-S-methyl, amisulbrom, anilazin, blasticidin-S, captafol, captan, chinomethionat, dazomet, debacarb, diclomezine, difenzoquat, difenzoquatmethylsulfate, fenoxanil, Folpet, oxolinic acid, piperalin, proquinazid, pyroquilon, quinoxyfen, triazoxide, tricyclazole, 2-butoxy-6-iodo-3-propylchromen-4-one, 5-chloro-1-(4,6-dimethoxy-pyrimidin-2-yl)-2-methyl-1H-benzoimidazole, 5-chloro-7-(4-methyl-piperidin-1-yl)-6-(2,4,6-trifluorophenyl)-[1,2,4]triazolo[1,5-a]pyrimidine, 6-(3,4-dichloro-phenyl)-5-methyl-[1,2,4]triazolo[1,5-a]pyrimidine-7-ylamine, 6-(4-tert-butylphenyl)-5-methyl-[1,2,4]triazolo[1,5-a]pyrimidine-7-ylamine, 5-methyl-6-(3,5,5-trimethyl-hexyl)-[1,2,4]triazolo[1,5-a]pyrimidine-7-ylamine, 5-methyl-6-octyl-[1,2,4]triazolo[1,5-a]pyrimidine-7-ylamine, 6-methyl-5-octyl-[1,2,4]triazolo[1,5-a]pyrimidine-7-ylamine, 6-ethyl-5-octyl-[1,2,4]triazolo[1,5-a]pyrimidine-7-ylamine, 5-ethyl-6-octyl-[1,2,4]triazolo[1,5-a]pyrimidine-7-ylamine, 5-ethyl-6-(3,5,5-trimethyl-hexyl)-[1,2,4]triazolo[1,5-a]pyrimidine-7-ylamine, 6-octyl-5-propyl-[1,2,4]triazolo[1,5-a]pyrimidine-7-ylamine, 5-methoxymethyl-6-octyl-[1,2,4]triazolo[1,5-a]pyrimidine-7-ylamine, 6-octyl-5-trifluoromethyl-[1,2,4]triazolo[1,5-a]pyrimidine-7-ylamine and 5-trifluoromethyl-6-(3,5,5-trimethylhexyl)-[1,2,4]triazolo[1,5-a]pyrimidine-7-ylamine;

B.5) Carbamates

B.5.1) thio- and dithiocarbamates: ferbam, mancozeb, maneb, metam, methasuiphocarb, metiram, propineb, thiram, zineb, ziram;

B.5.2) carbamates: benthiavalicarb, diethofencarb, flubenthiavalicarb, iprovalicarb, propamocarb, propamocarb hydrochlorid, valiphenal and N-(1-(1-(4-cyano-phenyl)-ethanesulfonyl)-but-2-yl) carbamic acid-(4-fluorophenyl) ester;

B.6) Other active substances

B.6.1) guanidines: guanidine, dodine, dodine free base, guazatine, guazatine-acetate, iminoctadine, iminoctadine-triacetate, iminoctadine-tris(albesilate);

B.6.2) antibiotics: kasugamycin, kasugamycin hydrochloride-hydrate, streptomycin, polyoxine, validamycin A;

B.6.3) nitrophenyl derivates: binapacryl, dinobuton, dinocap, nitrthal-isopropyl, tecnazen,

organometal compounds: fentin salts, such as fentin-acetate, fentin chloride or fentin hydroxide;

B.6.4) sulfur-containing heterocyclyl compounds: dithianon, isoprothiolane;

B.6.5) organophosphorus compounds: edifenphos, fosetyl, fosetyl-aluminum, iprobenfos, phosphorous acid and its salts, pyrazophos, tolclofos-methyl;

B.6.6) organochlorine compounds: chlorothalonil, dichlofluanid, dichlorophen, flusulfamide, hexachlorobenzene, pencycuron, pentachlorphenole and its salts, phthalide, quintozene, thiophanate-methyl, tolylfluanid, N-(4-chloro-2-nitro-phenyl)-N-ethyl-4-methyl-benzenesulfonamide;

B.6.7) inorganic active substances: Bordeaux mixture, copper acetate, copper hydroxide, copper oxychloride, basic copper sulfate, sulfur;

B.6.8) others: biphenyl, bronopol, cyflufenamid, cymoxanil, diphenylamin, metrafenone, mildiomycin, oxin-copper, prohexadione-calcium, spiroxamine, tolylfluanid, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2-methyl-4-(3-trimethylsilanyl-propoxy)-phenyl)-N-ethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetylypiperidin-4-yl}-thiazole-4-carboxylic acid methyl-(R)-1,2,3,4-tetrahydro-naphthalen-1-yl-amide, acetic acid 6-tert.-butyl-8-fluoro-2,3-dimethyl-quinolin-4-ylester and methoxy-acetic acid 6-tert-butyl-8-fluoro-2,3-dimethyl-quinolin-4-ylester.

B.7) Growth regulators

abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid , maleic hydrazide, mefluidide, mepiquat (mepiquat chloride), naphthaleneacetic acid, N-6-benzyladenine, paclobutrazol, prohexadione (prohexadione-calcium), prohydrojasmon, thidiazuron, triapenthenol, tributyl phosphorotrithioate, 2,3,5-tri-iodobenzoic acid , trinexapac-ethyl and uniconazole;

B.8) Herbicides

B.8.1) acetamides: acetochlor, alachlor, butachlor, dimethachlor, dimethenamid, flufenacet, mefenacet, metolachlor, metazachlor, napropamide, naproanilide, pethoxamid, pretilachior, propachior, thenyichior;

B.8.2) amino acid derivatives: bilanafos, glyphosate, glufosinate, sulfosate;

B.8.3) aryloxyphenoxypropionates: clodinafop, cyhalofop-butyl, fenoxaprop, fluazifop, haloxyfop, metamifop, propaquizafop, quizalofop, quizalofop-P-tefuryl;

B.8.4) Bipyridyls: diquat, paraquat;

B.8.5) (thio)carbamates: asulam, butylate, carbetamide, desmedipham, dimepiperate, eptam (EPTC), esprocarb, molinate, orbencarb, phenmedipham, prosulfocarb, pyributicarb, thiobencarb, triallate;

B.8.6) cyclohexanediones: butroxydim, clethodim, cycloxydim, profoxydim, sethoxydim, tepraloxydim, tralkoxydim;

B.8.7) dinitroanilines: benfluralin, ethalfuralin, oryzalin, pendimethalin, prodiamine, trifluralin;

B.8.8) diphenyl ethers: acifluorfen, aclonifen, bifenox, diclofop, ethoxyfen, fomesafen, lactofen, oxyfluorfen;

B.8.9) hydroxybenzonitriles: bomoxynil, dichlobenil, ioxynil;

B.8.9) imidazolinones: imazamethabenz, imazamox, imazapic, imazapyr, imazaquin, imazethapyr;

B.8.10) phenoxy acetic acids: clomeprop, 2,4-dichlorophenoxyacetic acid (2,4-D), 2,4-DB, dichiorprop, MCPA, MCPA-thioethyl, MCPB, Mecoprop;

B.8.11) pyrazines: chloridazon, flufenpyr-ethyl, fluthiacet, norfiurazon, pyridate;

B.8.12) pyridines: aminopyralid, clopyralid, diflufenican, dithiopyr, fluridone, fluroxypyr, picloram, picolinafen, thiazopyr;

B.8.13) sulfonyl ureas: amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, 1-((2-chloro-6-propyl-imidazo[1,2-b]pyridazin-3-yl)sulfonyl)-3-(4,6-dimethoxy-pyrimidin-2-yl)urea;

B.8.14) triazines: ametryn, atrazine, cyanazine, dimethametryn, ethiozin, hexazinone, metamitron, metribuzin, prometryn, simazine, terbuthylazine, terbutryn, triaziflam;

B.8.15) ureas: chlorotoluron, daimuron, diuron, fluometuron, isoproturon, linuron, methabenzthiazuron, tebuthiuron;

B.8.16) other acetolactate synthase inhibitors: bispyribac-sodium, cloransulam-methyl, diclosulam, florasulam, flucarbazone, fiumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, pyroxsulam;

B.8.17) others: amicarbazone, aminotriazole, anilofos, beflubutamid, benazolin, bencarbazone,benfluresate, benzofenap, bentazone, benzobicyclon, bromacil, bromobutide, butafenacil, butamifos, cafenstrole, carfentrazone, cinidon-ethlyl, chiorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, flurochioridone, flurtamone, indanofan, isoxaben, isoxaflutole, lenacil, propanil, propyzamide, quinclorac, quinmerac, mesotrione, methyl arsonic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamine, saflufenacil, sulcotrione, sulfentrazone, terbacil, tefuryltrione, tembotrione, thiencarbazone, topramezone, 4-hydroxy-3-[2-(2-methoxy-ethoxymethyl)-6-trifluoromethyl-pyridine-3-carbonyl]-bicyclo[3.2.1]oct-3-en-2-one, (3-[2-chloro-4-fluoro-5-(3-methyl-2,6-dioxo-4-trifluoromethyl-3,6-dihydro-2H-pyrimidin-1-yl)-phenoxy]-pyridin-2-yloxy)-acetic acid ethyl ester, 6-amino-5-chloro-2-cyclopropyl-pyrimidine-4-carboxylic acid methyl ester, 6-chloro-3-(2-cyclopropyl-6-methyl-phenoxy)-pyridazin-4-ol, 4-amino-3-chloro-6-(4-chloro-phenyl)-5-fluoro-pyridine-2-carboxylic acid, 4-amino-3-chloro-6-(4-chloro-2-fluoro-3-methoxy-phenyl)-pyridine-2-carboxylic acid methyl ester, and 4-amino-3-chloro-6-(4-chloro-3-dimethylamino-2-fluoro-phenyl)-pyridine-2-carboxylic acid methyl ester.

B.9) Insecticides

B.9.1) organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, suiprophos, tetrachlorvinphos, terbufos, triazophos, trichlorfon;

B.9.2) carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;

B.9.3) pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;

B.9.4) insect growth regulators: a) chitin synthesis inhibitors: benzoylureas: chlorfluazuron, cyramazin, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramat;

B.9.5) nicotinic receptor agonists/antagonists compounds: clothianidin, dinotefuran, imidacloprid, thiamethoxam, nitenpyram, acetamiprid, thiacloprid, 1-(2-chloro-thiazol-5-ylmethyl)-2-nitrimino-3,5-dimethyl-[1,3,5]triazinane;

B.9.6) GABA antagonist compounds: endosulfan, ethiprole, fipronil, vaniliprole, pyrafluprole, pyriprole, 5-amino-1-(2,6-dichloro-4-methyl-phenyl)-4-sulfinamoyl-1H-pyrazole-3-carbothioic acid amide;

B.9.7) macrocyclic lactone insecticides: abamectin, emamectin, milbemectin, lepimectin, spinosad, spinetoram;

B.9.8) mitochondrial electron transport inhibitor (METI) I acaricides: fenazaquin, pyridaben, tebufenpyrad, tolfenpyrad, flufenerim;

B.9.9) METI II and III compounds: acequinocyl, fluacyprim, hydramethylnon;

B.9.10) Uncouplers: chlorfenapyr;

B.9.11) oxidative phosphorylation inhibitors: cyhexatin, diafenthiuron, fenbutatin oxide, propargite;

B.9.12) moulting disruptor compounds: cryomazine;

B.9.13) mixed function oxidase inhibitors: piperonyl butoxide;

B.9.14) sodium channel blockers: indoxacarb, metaflumizone;

B.9.15) others: benclothiaz, bifenazate, cartap, flonicamid, pyridalyl, pymetrozine, sulfur, thiocyclam, flubendiamide, chlorantraniliprole, cyazypyr (HGW86), cyenopyrafen, flupyrazofos, cyflumetofen, amidoflumet, imicyafos, bistrifluron, and pyrifluquinazon.

B.10) Ethylen modulators

B.10.1) ethylene biosynthesis inhibitors which inhibit the conversion of S-adenosyl-L-methionine into 1-aminocyclopropane-1-carboxylic acid (ACC), such as derivatives of vinyiglycine, hydroxylamines, oxime ether derivatives;

B.10.2) inhibitors of ethylene biosynthesis which block the conversion of ACC into ethylene selected from Co++ or Ni++ ions, radical-scavenging phenolic substances such as n-propyl gallate, polyamines such as putrescine, spermine, spermidine, structural ACC analogs such as α-aminoisobutyric acid, L-aminocyclopropene-1-carboxylic acid, salicylic acid including its synthetic analogon acibenzolar-S-methyl, and also triazolyl compounds as inhibitors of cytochrome P-450-dependent monooxygenase;

B.10.3) inhibitors of the action of ethylene selected of the group consisting of: structural analogues of ethylene such as cyclopropene and its derivatives (i.e. U.S. Pat. Nos. 5,518,988, 6,194,350), specifically 1-methylcyclopropene (1-MCP), or 2,5-norbornadiene, and 3-amino-1,2,4-triazole or Ag+ ions.

A preferred embodiment of the method according to the invention, comprises the application of a mixture comprising at least one compound (A) of the formula I or an agriculturally useful salt thereof, and at least one agriculturally active compound (B) or an agriculturally useful salt thereof selected from the group consisting of

N-(3′,4′,5′-trifluorobiphenyl-2-yl)-1-methyl-3-difluoromethyl-1H-pyrazole-4-carboxamide, azoxystrobin, dimoxystrobin, fluoxastrobin, kresoxim-methyl, orysastrobin, picoxystrobin, pyraclostrobin, trifloxystrobin, bixafen, boscalid, isopyrazam, isotianil, metalaxyl, metalaxyl-M (mefenoxam), penthiopyrad, dimetomorph, zoxamide, mandipropamid, silthiofarm, cyproconazol, difenoconazole, epoxiconazole, fluquinconazole, flusilazole, flutriafol, metconazole, propiconazole, prothioconazole, tebuconazole, triticonazole, prochloraz, cyprodinil, pyrimethanil, fludioxonil, fenpropimorph, fenpropidin, iprodione, famoxadone, probenazole, proquinazid, quinoxifen, acibenzolar-S-methyl, metiram, thiram, guazatine, dithianon, fosetyl-aluminium, chlorothalonil, phthalide, thiophanate-methyl, cymoxanil, metrafenone, spiroxamine, chlormequat (chlormequat chloride), cyclanilide, ethephon, mepiquat (mepiquat chloride), N-6-benzyladenine, prohexadione (prohexadione-calcium), trinexapac-ethyl, 1-naphtaleneacetamide, dimethachlor, dimethenamid, flufenacet, mefenacet, meta-zachlor, glyphosate, glufosinate, fenoxaprop, fluazifop, propaquizafop, quizalofop-P-tefuryl, desmedipham, phenmedipham, prosulfocarb, clethodim, cycloxidim, sethoxidim, tepraloxidim, tralkoxydim, pendimethalin, acifluorfen, bifenox, bromoxynil, ioxynil, imazamethabenz, imazamox, imazapic, imazapyr, imazaquin, imazethapyr, 2,4 D, dichlorprop, MCPA, mecoprop, chloridazon, clopyralid, diflufenican, fluroxypyr, picloram, picolinafen, amidosulfuron, flupyrsulfuron, foramsulfuron, iodosulfuron, metsulfuron-methyl, nicosulfuron, prosulfuron, rimsulfuron, sulfosulfuron, thifensulfuron, triasulfuron, tribenuron, tritosulfuron, atrazine, metamitron, metribuzin, simazine, terbuthylazine, chlortoluron, isoproturon, florasulam, bentazone, clomazone, dicamba, diflufenzopyr, ethofumesate, isoxaflutole, propyzamide, quinclorac, quinmerac, mesotrione, sulcotrione, topremazone, triclopyr, pyridat, thiencarbazone, tefluryltrione, dimethoate, methiocarb, pirimicarb, bifenthrin, cyfluthrin, cyhalothrin, lambda-cyhalothrin, cypermethrin, alpha-cypermetrin, beta-cypermethrin, zeta-cypermethrin, deltamethrin, esfenvalerate, tefluthrin, clothianidin, imidacloprid, thiamethoxam, acetamiprid, thiacloprid, ethiprole, fipronil, metaflumizone, flonicamid, pymetrozine, chlorantriliprole, derivatives of vinylglycine, hydroxylamines, oxime ether derivatives, n-propyl gallate, polyamines such as putrescine, spermine, spermidine, salicylic acid including its synthetic analogon acibenzolar-S-methyl, and also triazolyl compounds, cyclopropene and its derivatives, 1-methylcyclopropene (1-MCP), 2,5-norbornadiene, and 3-amino-1,2,4-triazole or Ag+ ions.

Especially preferred compounds (B) according to the invention are those selected from the group consisting of:

N-(3′,4′,5′-trifluorobiphenyl-2-yl)-1-methyl-3-difluoromethyl-1H-pyrazole-4-carboxamide, azoxystrobin, dimoxystrobin, fluoxastrobin, kresoxim-methyl, orysastrobin, picoxystrobin, pyraclostrobin, trifloxystrobin, bixafen, boscalid, isopyrazam, penthiopyrad, dimethomorph, cyproconazol, difenoconazole, epoxiconazole, metconazole, propiconazole, prothioconazole, tebuconazole, metrafenone, chlormequat (chlormequat chloride), cyclanilide, ethephon, mepiquat (mepiquat chloride), trinexapac-ethyl, meta-zachlor, glyphosate, glufosinate, cyclohexanediones: cycloxidim, tepraloxidim, imidazolinones: imazamethabenz, imazamox, imazapic, imazapyr, imazaquin, imazethapyr, 2,4 D, dichlorprop, MCPA, Mecoprop, dicamba, diflufenzopyr, clothianidin, imidacloprid, thiamethoxam, fipronil, salicylic acid including its synthetic analogon acibenzolar-S-methyl, prohexadione-Ca or trinexapac-ethyl, structural analogues of ethylene such as cyclopropene and its derivatives, specifically 1-methylcyclopropene (1-MCP).

Most preferred compounds (B) according to the invention are those selected from the group consisting of:

N-(3′,4′,5′-trifluorobiphenyl-2-yl)-1-methyl-3-difluoromethyl-1H-pyrazole-4-carboxamide, azoxystrobin, kresoxim-methyl, orysastrobin, pyraclostrobin, bixafen, boscalid, isopyrazam, penthiopyrad, dimethomorph, epoxiconazole, metconazole, prothioconazole, tebuconazole, chlormequat (chlorme-quat chloride), ethephon, mepiquat (mepiquat chloride), prohexadione (prohexadione-calcium), trinexapac-ethyl, glyphosate, dicamba, diflufenzopyr, clothianidin, imidacloprid, thiamethoxam, fipronil, cyclopropene and its derivatives (such as 1-methylcyclopropene (1-MCP).

The active substances referred to as compound (B) as well as their prepartion are known from prior art (cf.: http://www.alanwood.net/pesticides/ as well as cf.: Tomlin, The Pesticide Manual, 14^{th} Ed., BCPC Publications 2006) and commercially available. The compounds described by IUPAC nomenclature are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624).

The composition used according to the invention can be a binary composition or a ternary or an even higher composition.

In the terms of the present invention, “binary compositions” are understood to comprise one compound (A) of the formula I and either a second compound (A) of formula I or one compound (B).

In the terms of the present invention, “ternary compositions” are understood to comprise one, two or three compounds (A) of formula I or two compounds (A) of the formula I and one compound (B) or one compound (A) of the formula 1 and two compounds (B).

In one embodiment, the composition used according to the invention comprises one compound (A) of formula I selected from the group consisting of Dimethyl-carbamic acid 2-methylcarbamoyl-2-[(1-methyl-3-trifluoromethyl-1H-pyrazole-4-carbonyl)-amino]-1-(tetrahydro-pyran-4-yl)-ethyl ester (compound 1), 5-Methyl-2-trifluoromethyl-furan-3-carboxylic acid (1-methylcarbamoyl-2-phenyl-ethyl)-amide (compound 2), 1-Methyl-3-trifluoromethyl-1H-pyrazole-4-carboxylic acid (1-methylcarbamoyl-2-phenyl-ethyl)-amide (compound 3), Methyl-carbamic acid-2-methylcarbamoyl-2-[(1-methyl-3-trifluoromethyl-1H-pyrazole-4-carbonyl)-amino]-ethyl ester (compound 4), Methyl-carbamic acid 2-methylcarbamoyl-2-[(1-methyl-3-trifluoromethyl-1H-pyrazole-4-carbonyl)-amino]-1-(tetrahydro-pyran-4-yl)-ethyl ester (compound 5), Methyl-carbamic acid 1,1-dimethyl-2-methylcarbamoyl-2-[(1-methyl-3-trifluoromethyl-1H-pyrazole-4-carbonyl)-amino]-ethyl ester (compound 6), 1-Methyl-3-trifluoromethyl-1H-pyrazole-4-carboxylic acid (2-hydroxy-3-methoxy-2-methoxymethyl-1-methylcarbamoyl-propyl)-amide (compound 7), Methyl-carbamic acid 1,1-bis-methoxymethyl-2-methylcarbamoyl-2-[(1-methyl-3-trifluoromethyl-1H-pyrazole-4-carbonyl)-amino]-ethyl ester (compound 8), Methyl-carbamic acid (E)-1-methyl-1-{methylcarbamoyl-[(1-methyl-3-trifluoromethyl-1H-pyrazole-4-carbonyl)-amino]-methyl}-but-2-enyl ester (compound 9), 2-Methyl-5-trifluoromethyl-oxazole-4-carboxylic acid (1-methylcarbamoyl-2-phenyl-ethyl)-amide, 2-Trifluoromethyl-thiophene-3-carboxylic acid (1-methylcarbamoyl-2-phenyl-ethyl)-amide, Methyl-carbamic acid 1-methyl-2-methylcarbamoyl-2-[(5-methyl-2-trifluoromethyl-furan-3-carbonyl)-amino]-ethyl ester, Methyl-carbamic acid 1-methyl-2-methylcarbamoyl-2-[(1-methyl-3-trifluoromethyl-1H-pyrazole-4-carbonyl)-amino]-ethyl ester, and Methyl-carbamic acid 1,1-dimethyl-2-methylcarbamoyl-2-[(5-methyl-2-trifluoromethyl-furan-3-carbonyl)-amino]-ethyl ester and one compound (B) selected from the group consisting of N-(3′,4′,5′-trifluorobiphenyl-2-yl)-1-methyl-3-difluoromethyl-1H-pyrazole-4-carboxamide, azoxystrobin, kresoxim-methyl, orysastrobin, pyraclostrobin, bixafen, boscalid, isopyrazam, penthiopyrad, dimetomorph, epoxiconazole, metconazole, prothioconazole, tebuconazole, chlormequat (chlormequat chloride), ethephon, mepiquat (mepiquat chloride), prohexadione (prohexadione-calcium), trinexapac-ethyl, glyphosate, dicamba, diflufenzopyr, clothianidin, imidacloprid, thiamethoxam, fipronil and cyclopropene and its derivatives (such as 1-methylcyclopropene (1-MCP).

In one embodiment, the method according to the invention comprises applying a mixture comprising at least one compound (A) of the formula I or an agriculturally useful salt thereof, and at least one compound (B) or an agriculturally useful salt thereof selected from N-(3′,4′,5′-trifluorobiphenyl-2-yl)-1-methyl-3-difluoromethyl-1H-pyrazole-4-carboxamide, azoxystrobin, kresoxim-methyl, orysastrobin, pyraclostrobin, bixafen, boscalid, isopyrazam, penthiopyrad, dimethomorph, epoxiconazole, metconazole, prothioconazole, tebuconazole, chlormequat (chlormequat chloride), ethephon, mepiquat (mepiquat chloride), prohexadione (prohexadione-calcium), trinexapac-ethyl, glyphosate; dicamba, diflufenzopyr, clothianidin, imidacloprid, thiamethoxam, fipronil and cyclopropene and its derivatives (such as 1-methylcyclopropene (1-MCP).

In one embodiment, the compound(s) (A) of formula I and the further active compound(s) (B) are applied in a plant health effective amount, preferably in a weight ratio of from 1:1000 to 1000:1.

In another embodiment, the compound(s) (A) of formula I and the further active compound(s) (B) are applied in a plant health effective amount, preferably in a weight ratio of from 1:100 to 100:1.

In another embodiment, the compound(s) (A) of formula I and the further active compound(s) (B) are applied in a plant health effective amount, preferably in a weight ratio of from 1:50 to 50:1.

In another embodiment, the compound(s) (A) of formula I and the further active compound(s) (B) are applied in a plant health effective amount, preferably in a weight ratio of from 1:10 to 10:1.

The compositions are used for improving the health of plants when applied to plants, parts of plants, propagules of the plants or to their actual or intended locus of growth.

Thus, the invention relates to a method for improving the health of plants, which comprises treating the plant, a part of the plant, the locus where the plant is growing or is expected to grow, and/or the propagules from which the plant grows with the composition used according to the invention. According to this inventive method, the plant, the locus where the plant is growing or is expected to grow, and/or the propagules from which the plant grows are preferably treated simultaneously (together or separately) or subsequently with the compounds (A) and (B). The subsequent application is carried out with a time interval which allows a combined action of the two compounds. Preferably, the time interval for a subsequent application of compounds (A) and (B) ranges from a few seconds up to 3 months, preferably, from a few seconds up to 1 month, more preferably from a few seconds up to 2 weeks, even more preferably from a few seconds up to 3 days and in particular from a few seconds up to 24 hours.

In one embodiment of the invention, the compound(s) (A) of formula I are applied to the plant propagules as seed treatment (BBCH principle growth stage 00).

In one embodiment of the invention, the compound(s) (A) of formula I are applied at the BBCH principal growth stage 6 (flowering).

In one embodiment of the invention, the compound(s) (A) of formula I are applied at the BBCH principal growth stage 7 (development of fruit).

In one embodiment of the invention, the compound(s) (A) of formula I are applied at least two times to the plant and/or its propagules.

In one embodiment of the invention, the compound(s) (A) of formula I are applied at the BBCH principal growth stage 00 (seed treatment) and at the BBCH principal growth stage 2 (formation of side shoots/tillering).

In one embodiment of the invention, the compound(s) (A) of formula I are applied at the BBCH principal growth stage 00 (seed treatment) and at the BBCH principal growth stage 6 (flowering).

In one embodiment of the invention, the compound(s) (A) of formula I are applied at the

BBCH principal growth stage 00 (seed treatment) and at the BBCH principal growth stage 7 (development of fruit).

In one embodiment of the invention, the compound(s) (A) of formula I are applied at the BBCH principal growth stage 2 (formation of side shoots/tillering) as well as at the BBCH principal growth stage 3 (stem elongation).

In one embodiment of the invention, the compound(s) (A) of formula I are applied at the BBCH principal growth stage 2 (formation of side shoots/tillering) as well as at the BBCH principal growth stage 4 (development of harvestable vegetative plant parts).

In one embodiment of the invention, the compound(s) (A) of formula I are applied at the BBCH principal growth stage 00 (seed treatment), at the BBCH principal growth stage 2 (formation of side shoots/tillering) as well as at the BBCH principal growth stage 4 (development of harvestable vegetative plant parts).

In one embodiment of the invention, the compound(s) (A) of formula I are applied at the BBCH principal growth stage 00 (seed treatment), at the BBCH principal growth stage 6 (flowering) as well as at the BBCH principal growth stage 7 (development of fruit).

In one embodiment of the invention, the compound(s) (A) of formula I and a further active compound(s) (B) are applied to the plant propagules as seed treatment (BBCH principle growth stage 00).

In one embodiment of the invention, the compound(s) (A) of formula I and a further active compound(s) (B) are applied at the BBCH principal growth stage 6 (flowering).

In one embodiment of the invention, the compound(s) (A) of formula I and a further active compound(s) (B) are applied at the BBCH principal growth stage 7 (development of fruit).

In one embodiment of the invention the compound(s) (A) of formula I and a further active compound(s) (B) are applied at least two times to the plant and/or its propagules.

In one embodiment of the invention, the compound(s) (A) of formula I and a further active compound(s) (B) are applied at the BBCH principal growth stage 00 (seed treatment) and at the BBCH principal growth stage 2 (formation of side shoots/tillering).

In one embodiment of the invention, the compound(s) (A) of formula I and a further active compound(s) (B) are applied at the BBCH principal growth stage 00 (seed treatment) and at the BBCH principal growth stage 6 (flowering).

In one embodiment of the invention, the compound(s) (A) of formula I and a further active compound(s) (B) are applied at the BBCH principal growth stage 00 (seed treatment) and at the BBCH principal growth stage 7 (development of fruit).

In one embodiment of the invention, the compound(s) (A) of formula I and a further active compound(s) (B) are applied at the BBCH principal growth stage 2 (formation of side shoots/tillering) as well as at the BBCH principal growth stage 3 (stem elongation).

In one embodiment of the invention, the compound(s) (A) of formula I and a further active compound(s) (B) are applied at the BBCH principal growth stage 2 (formation of side shoots/tillering) as well as at the BBCH principal growth stage 4 (development of harvestable vegetative plant parts).

In one embodiment of the invention, the compound(s) (A) of formula I and a further active compound(s) (B) are applied at the BBCH principal growth stage 00 (seed treatment), at the BBCH principal growth stage 2 (formation of side shoots/tillering) as well as at the BBCH principal growth stage 4 (development of harvestable vegetative plant parts).

In one embodiment of the invention, the compound(s) (A) of formula I and a further active compound(s) (B) are applied at the BBCH principal growth stage 00 (seed treatment), at the BBCH principal growth stage 6 (flowering) as well as at the BBCH principal growth stage 7 (development of fruit).

The term “BBCH principal growth stage” refers to the extended BBCH-scale which is a system for a uniform coding of phenologically similar growth stages of all mono- and dicotyldedonous plant species in which the entire developmental cycle of the plants is subdivided into clearly recoginizable and distinguishable longer-lasting developmental phases. The abbreviation BBCH dervies from Biologische Bundesanstalt, Bundessortenamt and CHemical industry.

The term “later developmental stage” means that the plants have reached at least the BBCH principal growth stage 2 (formation of side shoots/tillering).

The term “effective and non-phytotoxic amount” means that certain compounds are used in a quantity which allows to obtain the desired effect but which does not give rise to any phytotoxic symptoms on the treated plant or on the plant grown from the treated propagule or treated soil.

The term “plant health effective amount” denotes an amount of compound (A) of the formula I or of the mixtures of compound (A) of formula I and a compound (B), which is sufficient for achieving plant health effects as defined hereinbelow (for example an increase in yield). More exemplary information about amounts, ways of application and suitable ratios to be used is given below. The skilled artisan is well aware of the fact that such an amount can vary in a broad range and is dependent on various factors, e.g. the treated cultivated plant or material and the climatic conditions.

The term “synergistically” means that the purely additive (in mathematical terms) plant health-increasing effects of a simultaneous, that is joint or separate application of compound (A) of the formula I and compound (B) or successive application of compound (A) of the formula I and compound (B) is surpassed by the application of a mixture according to the invention.

In the terms of the present invention, “agriculturally useful salts” are especially those cations and anions which do not have any adverse effect on the action of the compounds according to the invention such as a) suitable cations, which are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium as well as b) suitable anions of useful acid addition salts, which are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formiate, acetate, propionate and butyrate.

The term “germination” is defined as observable root growth development from the embryo.

The term “emergence” is defined as observable growth above the rooting medium surface (typically above soil surface).

The term “plant propagules” is to be understood as all types of plant propagation material. The term embraces seeds, grains, fruit, tubers, rhizomes, spores, cuttings, offshoots, meristem tissues, single and multiple plant cells and any other plant tissue from which a complete plant can be obtained. One particular propagule is seed.

The term “plant propagation material” is to be understood to denote all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil.

The term “cultivated plants” is to be understood as including plants which have been modified by breeding, mutagenesis or genetic engineering including but not limiting to agricultural biotech products on the market or in development.

The term “genetically modified plants” is to be understood as plants, which genetic material has been modified by the use of recombinant DNA techniques in a way that under natural circumstances it cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transtional modification of protein(s), oligo- or polypeptides e. g. by glycosylation or polymer additions such as prenylated, acetylated or farnesylated moieties or PEG moieties. Plants that have been modified by breeding, mutagenesis or genetic engineering, e.g. have been rendered tolerant to applications of specific classes of herbicides, such as hydroxyphenylpyruvate dioxygenase (HPPD) inhibitors; acetolactate synthase (ALS) inhibitors, such as sulfonyl ureas (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e. g. U.S. Pat. No. 6,222,100, WO 01/82685, WO 00/026390, WO 97/41218, WO 98/002526, WO 98/02527, WO 04/106529, WO 05/20673, WO 03/014357, WO 03/13225, WO 03/14356, WO 04/16073); enolpyruvylshikimate-3-phosphate synthase (EPSPS) inhibitors, such as glyphosate (see e.g. WO 92/00377); glutamine synthetase (GS) inhibitors, such as glufosinate (see e.g. EP-A 242 236, EP-A 242 246) or oxynil herbicides (see e. g. U.S. Pat. No. 5,559,024) as a result of conventional methods of breeding or genetic engineering. Several cultivated plants have been rendered tolerant to herbicides by conventional methods of breeding (mutagenesis), e. g. Clearfield® summer rape (Canola, BASF SE, Germany) being tolerant to imidazolinones, e. g. imazamox. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.) and LibertyLink® (glufosinate-tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as δ-endotoxins, e.g. CryIA(b), CryIA(c), CryIF, CryIF(a2), CryIIA(b), CryIIIA, CryIIIB(b1) or Cry9c; vegetative insecticidal proteins (VIP), e. g. VIP1, VIP2, VIP3 or VIP3A; insecticidal proteins of bacteria colonizing nematodes, e. g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e. g. WO 02/015701). Further examples of such toxins or genetically modified plants capable of synthesizing such toxins are disclosed, e. g., in EP-A 374 753, WO 93/007278, WO 95/34656, EP-A 427529, EP-A 451878, WO 03/18810 and WO 03/52073. The methods for producing such genetically modified plants are generally known to the person skilled in the art and are described, e. g. in the publications mentioned above. These insecticidal proteins contained in the genetically modified plants impart to the plants producing these proteins tolerance to harmful pests from all taxonomic groups of athropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme Phosphinothricin-N-Acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e. g. Agrisure® CB) and Bt176 from Syngenta Seeds SAS, France, (corn cultivars producing the Cry1Ab toxin and PAT enyzme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e.g. EP-A 392 225), plant disease resistance genes (e. g. potato cultivars, which express resistance genes acting against Phytophthora infestans derived from the mexican wild potato Solanum bulbocastanum) or T4-lysozym (e.g. potato cultivars capable of synthesizing these proteins with increased resistance against bacteria such as Erwinia amylvora). The methods for producing such genetically modified plants are generally known to the person skilled in the art and are described, e.g. in the publications mentioned above.

Furthermore, plants are also covered that are by the use of recombinant DNA techniques capable to synthesize one or more proteins to increase the productivity (e.g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of substances of content or new substances of content, specifically to improve human or animal nutrition, e. g. oil crops that produce health-promoting long-chain omega-3 fatty acids or unsaturated omega-9 fatty acids (e. g. Nexera® rape, DOW Agro Sciences, Canada).

Furthermore, plants are also covered that contain by the use of recombinant DNA techniques a modified amount of substances of content or new substances of content, specifically to improve raw material production, e.g. potatoes that produce increased amounts of amylopectin (e.g. Amfiora® potato, BASF SE, Germany).

The term “locus” is to be understood as any type of environment, soil, area or material where the plant is growing or intended to grow as well as the environmental conditions (such as temperature, water availability, radiation) that have an influence on the growth and development of the plant and/or its propagules.

The term “plant health” (health of a plant) is defined as a condition of the plant and/or its products which is determined by several aspects alone or in combination with each other such as yield (for example increased biomass and/or increased content of valuable ingredients), plant vigor (for example improved plant growth and/or greener leaves (“greening effect”)), quality (for example improved content or composition of certain ingredients) and tolerance to abiotic and/or biotic stress.

The above identified indicators for the health condition of a plant may be interdependent and may result from each other.

One indicator for the condition of the plant is its yield. “Yield” is to be understood as any plant product of economic value that is produced by the plant such as grains, fruits in the proper sense, vegetables, nuts, grains, seeds, wood (e.g. in the case of silviculture plants) or even flowers (e.g. in the case of gardening plants, ornamentals). The plant products may in addition be further utilized and/or processed after harvesting.

According to the present invention, “increased yield” of a plant, in particular of an agricultural, silvicultural and/or ornamental plant means that the yield of a product of the respective plant is increased by a measurable amount over the yield of the same product of the plant produced under the same conditions, but without the application of the composition of the invention. Increased yield can be characterized, among others, by following improved properties of the plant:
increased plant weight,
increased plant height,
increased biomass such as higher fresh and/or dry weight
higher grain yield
more tillers
larger leaves
increased shoot growth
increased protein content
increased oil content
increased starch content
increased pigment content

According to one embodiment of the present invention, the yield is increased by at least 5%.

According to another embodiment of the present invention, the yield is increased by least 10%.

According to another embodiment of the present invention, the yield is increased by least 15%.

According to another embodiment of the present invention, the yield is increased by least 30%.

According to another embodiment of the present invention, the yield is increased by least 40%.

Another indicator for the condition of the plant is the “plant vigor”. The plant vigor becomes manifest in several aspects such as the general visual appearance. Improved plant vigor can be characterized, among others, by following improved properties of the plant:
improved vitality of the plant,
improved plant growth,
improved plant development,
improved visual appearance,
improved plant stand (less plant verse/lodging),
improved emergence,
enhanced root growth and/or more developed root system,
enhanced nodulation, in particular rhizobial nodulation,
bigger leaf blade,
bigger size,
increased plant weight,
increased plant height,
increased tiller number,
increased shoot growth,
increased root growth (extensive root system),
increased yield when grown on poor soils or unfavorable climate,
enhanced photosynthetic activity
enhanced pigment content (e.g. Chlorophyll content)
earlier flowering,
earlier fruiting,
earlier and improved germination,
earlier grain maturity,
improved self-defence mechanisms
improved stress tolerance and resistance of the plants against biotic and abiotic stress factors such as fungi, bacteria, viruses, insects, heat stress, cold stress, drought stress, UV stress and/or salt stress
less non-productive tillers,
less dead basal leaves,
less input needed (such as fertilizers or water)
greener leaves
complete maturation under shortened vegetation periods
less fertilizers needed,
less seeds needed,
easier harvesting
faster and more uniform ripening
longer shelf-life
longer panicles,
delay of senescence,
stronger and/or more productive tillers,
better extractability of ingredients
improved quality of seeds (for being seeded in the following seasons for seed production)
reduced production of ethylene and/or the inhibition of its reception by the plant

The improvement of the plant vigor according to the present invention particularly means that the improvement of any one or several or all of the above mentioned plant characteristics are improved independently of the pesticidal action of the composition or active ingredients.

Another indicator for the condition of the plant is the “quality” of a plant and/or the products of the respective plant. According to the present invention, enhanced quality means that certain crop characteristics such as the content or composition of certain ingredients are increased or improved by a measurable or noticeable amount over the same factor of the plant produced under the same conditions, but without the application of the composition of the present invention. The quality of a product of the respective plant becomes manifest in several aspects. Enhanced quality can be characterized, among others, by following improved properties of the plant or its product:
increased nutrient content
increased protein content
increased content of fatty acids.
increased metabolite content
increased carotenoid content
increased sugar content
increased amount of essential amino acids
improved nutrient composition
improved protein composition
improved composition of fatty acids
improved metabolite composition
improved carotenoid composition
improved sugar composition
improved amino acids composition
improved or optimal fruit color
improved leaf color
higher storage capacity
higher processability of the harvested products

Another indicator for the condition of the plant is the plant's tolerance or resistance to biotic and/or abiotic stress factors. Biotic and abiotic stress, especially over longer terms, can have harmful effects on plants. Biotic stress is caused by living organisms while abiotic stress is caused for example by environmental extremes. According to the present invention, “enhanced tolerance or resistance to biotic and/or abiotic stress factors” means (1.) that certain negative factors caused by biotic and/or abiotic stress are diminished in a measurable or noticeable amount as compared to plants exposed to the same conditions, but without being treated with the composition of the invention and (2.) that the negative effects are not diminished by a direct action of the composition on the stress factors, e.g. by its fungicidal or insecticidal action which directly destroys the microorganisms or pests, but rather by a stimulation of the plants' own defensive reactions against said stress factors.

Negative factors caused by biotic stress such as pathogens and pests are widely known and range from dotted leaves to total destruction of the plant. Biotic stress can be caused by living organisms, such as:
pests (for example insects, arachnides, nematodes),
competing plants (for example weeds),
microorganisms such as bacteria,
phythopathogenic fungi
viruses

Negative factors caused by abiotic stress are also well-known and can often be observed as reduced plant vigor (see above), for example dotted leaves, “burned leaves”, reduced growth, less flowers, less biomass, less crop yields, reduced nutritional value of the crops, later crop maturity, to give just a few examples. Abiotic stress can be caused for example by:
extremes in temperature such as heat or cold (heat stress/cold stress),
strong variations in temperature,
temperatures unusual for the specific season,
drought (drought stress),
extreme wetness,
high salinity (salt stress),
radiation (for example by increased UV radiation due to the decreasing ozone layer),
increased ozone levels (ozone stress)
organic pollution (for example by phythotoxic amounts of pesticides)
inorganic pollution (for example by heavy metal contaminants).

As a result of biotic and/or abiotic stress factors, the quantity and the quality of the stressed plants, their crops and fruits decrease. As far as quality is concerned, reproductive development is usually severely affected with consequences on the crops which are important for fruits or seeds. Synthesis, accumulation and storage of proteins are mostly affected by temperature; growth is slowed by almost all types of stress; polysaccharide synthesis, both structural and storage is reduced or modified: these effects bring to a decrease in biomass (yield) and to changes in the nutritional value of the product.

Advantageous properties, obtained especially from treated seeds, are e.g. improved germination and field establishment, better vigor and/or more homogen field establishment leeding finally to an increase of plant health such as an increased yield.

The above identified indicators for the health condition of a plant may be interdependent and may result from each other. For example, an increased resistance to biotic and/or abiotic stress may lead to a better plant vigor, e.g. to better and bigger crops, and thus to an increased yield. Inversely, a more developed root system may result in an increased resistance to biotic and/or abiotic stress. However, these interdependencies and interactions are neither all known nor fully understood and therefore the different indicators will be described separately.

The term “plants” is to be understood as plants of economic importance and/or men—grown plants such as crop and agricultural plants. Thus, they are preferably selected from agricultural, silvicultural and horticulural (including ornamental) plants.

In one embodiment according to the invention, the plant is selected from agricultural, silvicultural and horticultural plants, each in natural or genetically modified form.

In one embodiment of the invention, the plant health of which is to be improved by the treatment with the composition of the invention is an agricultural plant.

The term “agricultural plants” is to be understood as plants of which a part or all is harvested or cultivated on a commercial scale or which serve as an important source of feed, food, fibers (e.g. cotton, linen), combustibles (e.g. wood, bioethanol, biodiesel, iomass) or other chemical compounds. Agricultural plants also horticultural plants, i.e. plants grown in gardens (and not on fields), such as certain fruits and vegetables. Examples for agricultural plants are soybean, corn (maize), wheat, triticale, barley, oats, Brassica species, rye, rape, such as canola/oilseed rape, millet (sorghum), rice, sunflower, cotton, sugar beets, pome fruit, stone fruit, citrus, bananas, strawberries, blueberries, almonds, grapes, mango, papaya, peanuts, potatoes, tomatoes, peppers, cucurbits, cucumbers, melons, watermelons, garlic, onions, carrots, cabbage, beans, peas, lentils, alfalfa (lucerne), trefoil, clovers, flax, elephant grass (Miscanthus), grass, lettuce, sugar cane, tea, tobacco and coffee.

In another embodiment of the invention, the plant health of which is to be improved by the treatment with the composition of the invention is a silvicultural plant.

The term “silvicultural plants” is to be understood as trees, more specifically trees used in reforestation or industrial plantations. Industrial plantations generally serve for the commercial production of forest products, such as wood, pulp, paper, rubber tree, Christmas trees, or young trees for gardening purposes. Examples for silvicultural plants are conifers, like pines, in particular Pinus spp., fir and spruce, eucalyptus, tropical trees like teak, rubber tree, oil palm, willow (Salix), in particular Salix spp., poplar (cottonwood), in particular Populus spp., beech, in particular Fagus spp., birch, oil palm, and oak.

In another embodiment of the invention, the plant health of which is to be improved by the treatment with the composition of the invention is a horticultural plant.

The term “horticultural plants” are to be understood as plants which are commonly used in horticulture—e.g. the cultivation of ornamentals, vegetables and/or fruits. Ex-amples for ornamentals are turf, geranium, pelargonia, petunia, begonia, and fuchsia, to name just a few among the vast number of ornamentals. Examples for vegetables potatoes, tomatoes, peppers, cucurbits, cucumbers, melons, watermelons, garlic, onions, carrots, cabbage, beans, peas and lettuce and more preferably from tomatoes, onions, peas and lettuce, to name just a few among the vast number of vegetables. Examples for fruits are apples, pears, cherries, stawberry, citrus, peaches, apricots, blueberries, to name just a few among the vast number of fruits.

In one embodiment according to the invention, the plant is selected from alfalfa, annual grass, bamboo, barley, cacao, canola, castor-oil plant, coconut, coffee, cotton, eggplant, eucalyptus, flax, forage crops, grape, Jatropha, linseed, maize, manihot, manioc/cassava, Miscanthus species, oat, oil palm, Panicum species, pea, peanut, perennial grass, pine, poplar/cottonwood, potato, rapeseed, rice, rye, safflower, Salix species, silver maple, soybean, spruce, sugar beet, sugarcane, sunflower, sweet potato, tea, tobacco, tomato, triticale, Vicia species, grapes and wheat.

In another embodiment according to the invention, the plant is selected from apple, pear, peach, stawberry, potato, tomato, cururbits, grapes and Brassica species.

In another embodiment according to the invention, the plant is selected from barley, canola, cotton, Jatropha, maize, Miscanthus species, potato, rapeseed, rice, soybean, sugar beet, sugarcane, sunflower, grapes and wheat.

In another embodiment according to the invention, the plant is selected from pines, oil palms, rubber tree, poplar and Salix species.

The plants can be non-transgenic plants or can be plants that have at least one transgenic event. In one embodiment, the plant is a transgenic plant having a transgenic event that confers resistance to a pesticide. Examples for transgenic plants having a pesticide resistance are transgenic crops which are resistant to herbicides from the group consisting of the sulfonylureas (see for example EP-A-0257993, U.S. Pat. No. 5,013,659), imidazolinones (see for example U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073), glufosinate-type (see for example EP-A-0242236, EP-A-242246) or glyphosate-type (see for example WO 92/00377) or plants resistant towards herbicides selected from the group of cyclohexadienone/aryloxyphenoxypropionic acid herbicides (see for example U.S. Pat. No. 5,162,602, U.S. Pat No. 5,290,696, U.S. Pat. No. 5,498,544, U.S. Pat. No. 5,428,001, U.S. Pat. No. 6,069,298, U.S. Pat. No. 6,268,550, U.S. Pat. No. 6,146,867, U.S. Pat. No. 6,222,099, U.S. Pat. No. 6,414,222) or transgenic crop plants, for example cotton, with the capability of producing Bacillus thuringiensis toxins (Bt toxins) which make the plants resistant to certain pests (see for example EP-A-0142924, EP-A-0193259).

It is to be understood, however, that when the plant is a transgenic plant, the transgenic events that are present in the plant are by no means limited to those that provide pesticide resistance, but can include any transgenic event. In fact, the use of “stacked” transgenic events in a plant is also contemplated.

The compounds of formula I can be present in different crystal modifications whose biological activity may differ.

Plant propagation materials may be treated with at least one compound (A) of the formula I or a mixture of at least one compound (A) and at least one compound (B) as such or a composition comprising at least one compound (A) prophylactically either at or before planting or transplanting.

The invention also relates to agrochemical compositions comprising a solvent or solid carrier and at least one compound (A) and to the use for improving the health of a plant.

The compounds (A), and their salts can be converted into customary types of agro-chemical compositions, e.g. solutions, emulsions, suspensions, dusts, powders, pastes and granules. The composition type depends on the particular intended purpose; in each case, it should ensure a fine and uniform distribution of the compound according to the invention.

Examples for composition types are suspensions (SC, OD, FS), pastes, pastilles, wet-table powders or dusts (WP, SP, SS, WS, DP, DS) or granules (GR, FG, GG, MG), which can be water-soluble or wettable, as well as gel formulations for the treatment of plant propagation materials such as seeds (GF). Usually the composition types (e.g. SC, OD, FS, WG, SG, WP, SP, SS, WS, GF) are employed diluted. Composition types such as DP, DS, GR, FG, GG and MG are usually used undiluted. The compositions are prepared in a known manner (cf. U.S. Pat. No. 3,060,084, EP-A 707 445 (for liquid concentrates), Browning: “Agglomeration”, Chemical Engineering, Dec. 4, 1967, 147-48, Perry's Chemical Engineer's Handbook, 4th Ed., McGraw-Hill, New York, 1963, S. 8-57 and ff. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman: Weed Control as a Science (J. Wiley & Sons, New York, 1961), Hance et al.: Weed Control Handbook (8th Ed., Blackwell Scientific, Oxford, 1989) and Mollet, H. and Grubemann, A.: Formulation technology (Wiley VCH Verlag, Weinheim, 2001).

The agrochemical compositions may also comprise auxiliaries which are customary in agrochemical compositions. The auxiliaries used depend on the particular application form and active substance, respectively. Examples for suitable auxiliaries are solvents, solid carriers, dispersants or emulsifiers (such as further solubilizers, protective colloids, surfactants and adhesion agents), organic and anorganic thickeners, bactericides, anti-freezing agents, anti-foaming agents, if appropriate colorants and tackifiers or binders (e.g. for seed treatment formulations).

Suitable solvents are water, organic solvents such as mineral oil fractions of medium to high boiling point, such as kerosene or diesel oil, furthermore coal tar oils and oils of vegetable or animal origin, aliphatic, cyclic and aromatic hydrocarbons, e. g. toluene, xylene, paraffin, tetrahydronaphthalene, alkylated naphthalenes or their derivatives, alcohols such as methanol, ethanol, propanol, butanol and cyclohexanol, glycols, ketones such as cyclohexanone and gamma-butyrolactone, fatty acid dimethylamides, fatty acids and fatty acid esters and strongly polar solvents, e. g. amines such as N-methylpyrrolidone.

Solid carriers are mineral earths such as silicates, silica gels, talc, kaolins, limestone, lime, chalk, bole, loess, clays, dolomite, diatomaceous earth, calcium sulfate, magneslum sulfate, magnesium oxide, ground synthetic materials, fertilizers, such as, e.g., ammonium sulfate, ammonium phosphate, ammonium nitrate, ureas, and products of vegetable origin, such as cereal meal, tree bark meal, wood meal and nutshell meal, cellulose powders and other solid carriers.

Suitable surfactants (adjuvants, wtters, tackifiers, dispersants or emulsifiers) are alkali metal, alkaline earth metal and ammonium salts of aromatic sulfonic acids, such as ligninsoulfonic acid (Borresperse® types, Borregard, Norway) phenolsulfonic acid, naphthalenesulfonic acid (Morwet® types, Akzo Nobel, U.S.A.), dibutylnaphthalenesulfonic acid (Nekal® types, BASF, Germany),and fatty acids, alkylsulfonates, alkyl-arylsulfonates, alkyl sulfates, laurylether sulfates, fatty alcohol sulfates, and sulfated hexa-, hepta- and octadecanolates, sulfated fatty alcohol glycol ethers, furthermore condensates of naphthalene or of naphthalenesulfonic acid with phenol and formaldehyde, polyoxy-ethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and proteins, denatured proteins, polysaccharides (e.g. methylcellulose), hydrophobically modified starches, polyvinyl alcohols (Mowiol® types, Clariant, Switzerland), polycarboxylates (Sokolan® types, BASF, Germany), polyalkoxylates, polyvinylamines (Lupasol® types, BASF, Germany), polyvinylpyrrolidone and the copolymers thereof.

Examples for thickeners (i.e. compounds that impart a modified flowability to compositions, i. e. high viscosity under static conditions and low viscosity during agitation) are polysaccharides and organic and anorganic clays such as Xanthan gum (Kelzan®, CP Kelco, U.S.A.), Rhodopol®23 (Rhodia, France), Veegum® (R.T. Vanderbilt, U.S.A.) or Attaclay® (Engelhard Corp., NJ, USA).

Bactericides may be added for preservation and stabilization of the composition. Examples for suitable bactericides are those based on dichlorophene and benzylalcohol hemi formal (Proxel® from ICI or Acticide® RS from Thor Chemie and Kathon® MK from Rohm & Haas) and isothiazolinone derivatives such as alkylisothiazolinones and benzisothiazolinones (Acticide® MBS from Thor Chemie).

Examples for suitable anti-freezing agents are ethylene glycol, propylene glycol, urea and glycerin. Examples for anti-foaming agents are silicone emulsions (such as e.g. Silikon® SRE, Wacker, Germany or Rhodorsil®, Rhodia, France), long chain alcohols, fatty acids, salts of fatty acids, fluoroorganic compounds and mixtures thereof.

Suitable colorants are pigments of low water solubility and water-soluble dyes. Examples to be mentioned and the designations rhodamin B, C.I. pigment red 112, C.I. solvent red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples for tackifiers or binders are polyvinylpyrrolidons, polyvinylacetates, polyvinyl alcohols and cellulose ethers (Tylose®, Shin-Etsu, Japan).

Powders, materials for spreading and dusts can be prepared by mixing or concomitantly grinding the compounds I and, if appropriate, further active substances, with at least one solid carrier.

Granules, e.g. coated granules, impregnated granules and homogeneous granules, can be prepared by binding the active substances to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e. g., ammonium sulfate, ammonium phosphate, ammonium nitrate, ureas, and products of vegetable origin, such as cereal meal, tree bark meal, wood meal and nutshell meal, cellulose powders and other solid carriers.

Examples for composition types are:

1. Composition types for dilution with water

i) Water-soluble concentrates (SL, LS)

10 parts by weight of a compound I according to the invention are dissolved in 90 parts by weight of water or in a water-soluble solvent. As an alternative, wetting agents or other auxiliaries are added. The active substance dissolves upon dilution with water. In this way, a composition having a content of 10% by weight of active substance is obtained.

ii) Dispersible concentrates (DC)

20 parts by weight of a compound I according to the invention are dissolved in 70 parts by weight of cyclohexanone with addition of 10 parts by weight of a dispersant, e. g. polyvinylpyrrolidone. Dilution with water gives a dispersion. The active substance content is 20% by weight.

iii) Emulsifiable concentrates (EC)

15 parts by weight of a compound I according to the invention are dissolved in 75 parts by weight of xylene with addition of calcium dodecylbenzenesulfonate and castor oil ethoxylate (in each case 5 parts by weight). Dilution with water gives an emulsion. The composition has an active substance content of 15% by weight.

iv) Emulsions (EW, EO, ES)

25 parts by weight of a compound I according to the invention are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (Ultraturrax) and made into a homogeneous emulsion. Dilution with water gives an emulsion. The composition has an active substance content of 25% by weight.

v) Suspensions (SC, OD, FS)

In an agitated ball mill, 20 parts by weight of a compound I according to the invention are comminuted with addition of 10 parts by weight of dispersants and wetting agents and 70 parts by weight of water or an organic solvent to give a fine active substance suspension. Dilution with water gives a stable suspension of the active substance. The active substance content in the composition is 20% by weight.

vi) Water-dispersible granules and water-soluble granules (WG, SG)

50 parts by weight of a compound I according to the invention are ground finely with addition of 50 parts by weight of dispersants and wetting agents and prepared as water-dispersible or water-soluble granules by means of technical appliances (e. g. extrusion, spray tower, fluidized bed). Dilution with water gives a stable dispersion or solution of the active substance. The composition has an active substance content of 50% by weight.

vii) Water-dispersible powders and water-soluble powders (WP, SP, SS, WS)

75 parts by weight of a compound I according to the invention are ground in a rotorstator mill with addition of 25 parts by weight of dispersants, wetting agents and silica gel. Dilution with water gives a stable dispersion or solution of the active substance. The active substance content of the composition is 75% by weight.

viii) Gel (GF)

In an agitated ball mill, 20 parts by weight of a compound I according to the invention are comminuted with addition of 10 parts by weight of dispersants, 1 part by weight of a gelling agent wetters and 70 parts by weight of water or of an organic solvent to give a fine suspension of the active substance. Dilution with water gives a stable suspension of the active substance, whereby a composition with 20% (w/w) of active substance is obtained.

2. Composition types to be applied undiluted

ix) Dustable powders (DP, DS)

5 parts by weight of a compound I according to the invention are ground finely and mixed intimately with 95 parts by weight of finely divided kaolin. This gives a dustable composition having an active substance content of 5% by weight.

x) Granules (GR, FG, GG, MG)

0.5 parts by weight of a compound I according to the invention is ground finely and associated with 99.5 parts by weight of carriers. Current methods are extrusion, spray-drying or the fluidized bed. This gives granules to be applied undiluted having an active substance content of 0.5% by weight.

xi) ULV solutions (UL)

10 parts by weight of a compound I according to the invention are dissolved in 90 parts by weight of an organic solvent, e.g. xylene. This gives a composition to be applied undiluted having an active substance content of 10% by weight.

The agrochemical compositions generally comprise between 0.01 and 95%, preferably between 0.1 and 90%, most preferably between 0.5 and 90%, by weight of active substance. The active substances are employed in a purity of from 90% to 100%, preferably from 95% to 100% (according to NMR spectrum).

Water-soluble concentrates (LS), flowable concentrates (FS), powders for dry treatment (DS), water-dispersible powders for slurry treatment (WS), water-soluble powders (SS), emulsions (ES) emulsifiable concentrates (EC) and gels (GF) are usually employed for the purposes of treatment of plant propagation materials, particularly seeds. These compositions can be applied to plant propagation materials, particularly seeds, diluted or undiluted. The compositions in question give, after two-to-tenfold dilution, active substance concentrations of from 0.01 to 60% by weight, preferably from 0.1 to 40% by weight, in the ready-to-use preparations. Application can be carried out before or during sowing. Methods for applying or treating agrochemical compounds and compositions thereof, respectively, on to plant propagation material, especially seeds, are known in the art, and include dressing, coating, pelleting, dusting, soaking and in-furrow application methods of the propagation material. In a preferred embodiment, the compounds or the compositions thereof, respectively, are applied on to the plant propagation material by a method such that germination is not induced, e.g. by seed dressing, pelleting, coating and dusting.

In a preferred embodiment, a suspension-type (FS) composition is used for seed treatment. Typically, a FS composition may comprise 1-800 g/l of active substance, 1-200 g/l Surfactant, 0 to 200 g/l antifreezing agent, 0 to 400 g/l of binder, 0 to 200 g/l of a pigment and up to 1 liter of a solvent, preferably water.

The active substances can be used as such or in the form of their compositions, e.g. in the form of directly sprayable solutions, powders, suspensions, dispersions, emulsions, oil dispersions, pastes, dustable products, materials for spreading, or granules, by means of spraying, atomizing, dusting, spreading, brushing, immersing or pouring. The application forms depend entirely on the intended purposes; it is intended to ensure in each case the finest possible distribution of the active substances according to the invention.

Aqueous application forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active substance concentrations in the ready-to-use preparations can be varied within relatively wide ranges. In general, they are from 0.0001 to 10%, preferably from 0.001 to 1% by weight of active substance. The active substances may also be used successfully in the ultra-low-volume process (ULV), it being possible to apply compositions comprising over 95% by weight of active substance, or even to apply the active substance without additives.

As a matter of course, compounds (A) and (B) are used in an effective and non-phytotoxic amount.

The required application rate of the active ingredients (a.i.) according to the invention i.e. compound(s) (A) of the formula I or compound(s) (B) without formulation auxiliaries depends on multiple factors, among others on the plant stand, the soil, on the development stage of the plants, on the climatic conditions at the application site and on the application method. In general, the total amount in which compounds (A) or (B) are applied is from 0.001 g to 4 kg a.i./ha.

In one embodiment according to the invention, at least one compound (A) of the formula I or an agriculturally useful salt thereof is applied to the plant and/or the locus where the plant is growing or is intended to grow in an amount of from 0.001 g to 4 kg a.i./ha.

In one embodiment, when at least one compound (A) of the formula I or an agriculturally useful salt thereof is employed for improving the health of a plant, the amounts (dose rates) applied are between 0.01 g and 2 kg a.i./ha.

In another embodiment, when employed for improving the health of a plant, the amounts (dose rates) applied are between 0.1 g and 400 g a.i./ha.

In another embodiment, when employed for improving the health of a plant, the amounts (dose rates) applied are between 0.1 g and 200 g a.i./ha.

In another embodiment, when employed for improving the health of a plant, the amounts (dose rates) applied are between 0.1 g and 100 g a.i./ha.

In another embodiment, when employed for improving the health of a plant, the amounts (dose rates) applied are between 0.1 g and 50 g a.i./ha.

In another embodiment, when employed for improving the health of a plant, the amounts (dose rates) applied are between 0.1 g and 25 g a.i./ha.

In another embodiment, when employed for improving the health of a plant, the amounts (dose rates) applied are between 0.1 g and 10 g a.i./ha.

In another embodiment, when employed for improving the health of a plant, the amounts (dose rates) applied are between 0.1 g and 5 g a.i./ha.

In another embodiment, when employed for improving the health of a plant, the amounts (dose rates) applied are between 0.1 g and 2,5 g a.i./ha

In the treatment of plant propagation material such as seed, e.g. by dusting, coating or drenching seed, amounts of the active ingredient(s) of from 0.01 g to 4 kg per 100 kg of seed are required.

In another embodiment of the invention, the treatment of plant propagation material such as seed, e.g. by dusting, coating or drenching seed, amounts of the active ingredient(s) of from 0.01 g to 200 g per 100 kg of seed are required.

In yet another embodiment, treatment of plant propagation material such as seed, e.g. by dusting, coating or drenching seed, amounts of the active ingredient(s) of from 0.1 g to 100 g per 100 kg of seed are required.

Various types of oils, welters, adjuvants, herbicides, bactericides, other fungicides and/or pesticides may be added to the active substances or the compositions comprising them, if appropriate not until immediately prior to use (tank mix). These agents can be admixed with the compositions according to the invention in a weight ratio of 1:100 to 100:1, preferably 1:10 to 10:1 with the active compounds (A) and (B) employed according to the invention.

Adjuvants which can be used are in particular organic modified polysiloxanes such as Break Thru S 240®; alcohol alkoxylates such as Atplus 245®, Atplus MBA 1303®, Plurafac LF 300® and Lutensol ON 30®; EO/PO block polymers, e.g. Pluronic RPE 2035® and Genapol B®; alcohol ethoxylates such as Lutensol XP 80®; and dioctyl sulfosuccinate sodium such as Leophen RA®.

The methods of the invention are generally carried out by bringing the plant to be treated, parts of plant, the locus where the plant is growing or is intended to grow and/or its propagules in contact with the composition of the invention or with a formulation comprising it. To this end, the mixture or the individual active components (A) and (B) are applied to the plant, parts of plant, the locus where the plant is growing or is intended to grow and/or its propagules.

In one preferred embodiment, the method according to the invention is carried out as seed treatment.

For treating the propagules, in particular the seed (seed treatment), it is possible in principle to use any customary methods for treating or dressing seed, such as, but not limited to, seed dressing, seed coating, seed dusting, seed soaking, seed film coating, seed multilayer coating, seed encrusting, seed dripping, and seed pelleting. Specifically, the treatment is carried out by mixing the seed with the particular amount desired of seed dressing formulations either as such or after prior dilution with water in an apparatus suitable for this purpose, for example a mixing apparatus for solid or solid/liquid mixing partners, until the composition is distributed uniformly on the seed. If appropriate, this is followed by a drying operation.

Treatment of the propagules is in general only suitable for seasonal, in particular annual plants, i.e. for plants which are completely harvested after one season and which have to be replanted for the next season.

For treating the locus where the plant is growing or intended to grow, especially the soil, the latter may be treated a) before the propagule is planted/sowed b) at the time of planting or sowing along with the propagule (in case of seed sowing this is called in-furrow application) and/or c) after planting/sowing or even after germination of the plant with a suitable amount of the composition of the invention either as such or after prior dilution with water.

Soil application is for example a suitable method for cereals, cotton, sunflower and trees, in particular if growing in a plantation.

The inventive method is suitable for foliar application in living crops of plants, for soil applications prior to sowing or planting, including overall soil treatment and furrow applications, as well as, in particular, for dressing applications on plant propagation material. The latter term embraces seeds of all kinds (fruit, tubers, grains), cuttings, cut shoots and the like. One particular field of application is the treatment of all kinds of seeds.

In case the plants or (overground) parts thereof are to be treated, this is preferably done by spraying the plant or parts thereof, preferably their leaves (foliar application). Here, application can be carried out, for example, by customary spray techniques using spray liquor amounts of from about 100 to 1000 l/ha (for example from 300 to 400 l/ha) using water as carrier. Application of the composition of the invention by the low-volume and ultra-low-volume method is possible, as is their application in the form of microgranules.

In case of foliar treatment, the plants are treated after emergence of the plant. The optimum time for treatment depends on the specific plant species and can easily be determined by appropriate tests.

In one preferred embodiment, in the methods of the present invention, the propagules of the plants, in particular the seeds or the soil where the plants grow or are intended to grow are treated with the composition of the invention. If the soil is treated, this is preferably carried out as in-furrow application. Seed treatment is preferably carried out by the methods described above.

More preferably, in the methods of the present invention, the propagules of the plants, in particular the seeds are treated with the composition of the invention.

Combinations of specific or preferred embodiments with other specific or preferred embodiments are within the scope of the present invention.

The invention is now further illustrated by the following non-limiting examples.

### EXAMPLES

### Example 1

### Growth-Promoting Effect on Wheat Plants After Root Treatment

Wheat plants (Triticum aestivum cv. Cubus) at the 2nd leaf stage were cultivated in 320 mL glass vessels with 300 mL half-strength Linsmaier-Skoog medium (Physiologia Plantarum 18: 100-127, 1964). The solution was aerated throughout experiment. The vessels were incubated randomized in a climate chamber under 16:8 h light:dark cycles (photon flux density 400 μmol m-2 s-1, 400-750 nm, Osram powerstar HQI-R 250W/NDL and Osram krypton 100W lamps) at 22° C. (light):20° C. (dark) and 75% relative humidity (3 plants per vessel, 9 replicate vessels). After 2 days of adaptation, plants were treated with Dimethyl-carbamic acid 2-methylcarbamoyl-2-[(1-methyl-3-trifluoromethyl-1H-pyrazole-4-carbonyl)-amino]-1-(tetrahydro-pyran-4-yl)-ethyl ester (compound 1) which was added to the medium in dimethyl sulfoxide (DMSO) solution (0.1% final concentration of DMSO). Controls received corresponding quantity of DMSO alone, with no adverse effect on the growth of the plants. Seven days after treatment, fresh weights (FW) of shoot and root, as the growth parameters, were measured and mean values±standard error (SE) of 27 plant parts was calculated. Two separate experiments were carried out and their results are shown in table 22.

TABLE 22
Effects on shoot and root fresh weight

| | Shoot- | % of | | |
|---|---|---|---|---|
| Treatments | FW ± SE | control | Root-FW ± SE | % of control |
| Experiment 1 | | | | |
| Control | 0.652 ± 0.023 | 100.0 | 0.358 ± 0.012 | 100.0 |
| Compound 1 | | | | |
| 1 μM | 0.747 ± 0.017 | 114.6 | 0.403 ± 0.015 | 112.6 |
| 3 μM | 0.733 ± 0.019 | 112.4 | 0.382 ± 0.013 | 106.7 |
| Experiment 2 | | | | |
| Control | 0.640 ± 0.019 | 100.0 | 0.325 ± 0.010 | 100.0 |
| Compound 1 | | | | |
| 1 μM | 0.736 ± 0.024 | 115.0 | 0.342 ± 0.010 | 105.2 |
| 3 μM | 0.738 ± 0.021 | 115.3 | 0.335 ± 0.012 | 103.1 |

As shown in table 22, compound 1 increased yield by stimulating shoot and root growth of treated plants reproducibly by up to 15%, relative to controls. Concomitantly, shoot tillering and adventitious root growth was promoted.

### Example 2

### Growth-Promoting Effect on Wheat Plants After Foliar Treatment

Wheat plants (Triticum aestivum cv. Monopol) were grown in soil with ten plants per pot (13 cm diameter) under greenhouse conditions. Plants at the 2nd leaf stage were sprayed with an aqueous solution of formulated Dimethyl-carbamic acid 2-methylcarbamoyl-2-[(1-methyl-3-trifluoromethyl-1H-pyrazole-4-carbonyl)-amino]-1-(tetrahydro-pyran-4-yl)-ethyl ester (compound 1). The compound was applied at three different dose rates, i.e. 31.25/15.63/7.81 g ai/ha and the spray volume was 750 l/ha. Ten replicate pots with ten plants each were used per treatment. After three weeks (21 days) plants were harvested and growth parameters were determined. The recorded parameters were fresh weight, dry weigh, leaf area (all per pot) and number of tillers (per plant). Mean values of results, standard deviation (SD) and percentage to control were calculated. The results are listed in tables 23 and 24.

TABLE 23
Effects on shoot fresh weight (FW) and dry weight (DW)

| | Shoot | ±SD | % of | Shoot | ±SD | % of |
|---|---|---|---|---|---|---|
| Treatments | FW [g] | [g] | control | DW [g] | [g] | control |
| Control | 34.8 | 1.38 | 100.0 | 3.5 | 0.14 | 100.0 |
| Compound 1 | 42.8 | 2.43 | 123.2 | 4.2 | 0.25 | 121.7 |
| 31.25 g/ha | | | | | | |
| Compound 1 | 40.5 | 3.20 | 116.5 | 4.0 | 0.34 | 115.5 |
| 15.63 g/ha | | | | | | |
| Compound 1 | 43.9 | 2.62 | 126.2 | 4.4 | 0.27 | 126.6 |
| 7.81 g/ha | | | | | | |

As shown in table 23, compound 1 increased the yield by stimulating shoot growth, indicated by an increase in fresh and dry weight by up to 26% related to control. Furthermore, the results indicate a dose response, i.e. by lowering the dose rate (reducing the application rate), the growth promoting (yield increasing effect) became more pronounced.

TABLE 24
Effects on leaf area and number of tillers

| | Leaf area | ±SD | % of | Number | | % of |
|---|---|---|---|---|---|---|
| Treatments | [cm²] | [cm²] | control | of tillers | ±SD | control |
| Control | 887.6 | 46.01 | 100.0 | 2.4 | 0.57 | 100.0 |
| Compound 1 | 1060.3 | 86.11 | 119.5 | 2.8 | 0.43 | 113.5 |
| 31.25 g/ha | | | | | | |
| Compound 1 | 1046.3 | 125.87 | 117.9 | 2.8 | 0.50 | 114.1 |
| 15.63 g/ha | | | | | | |
| Compound 1 | 1121.1 | 153.68 | 126.3 | 2.9 | 0.35 | 117.9 |
| 7.81 g/ha | | | | | | |

As shown in table 24, compound 1 also increased leaf area, Again, the lowest dose rate resulted in the highest effect. Concomitantly, shoot tillering was increased.

### Example 3

### Growth-Promoting Effect on Wheat Plants After Foliar Treatment

Wheat plants (Triticum aestivum cv. Monopol) were grown in soil with ten plants per pot (13 cm diameter) under greenhouse conditions. Plants at the 2nd leaf stage were sprayed with aqueous solutions of formulated compounds:
Compound 2: 5-Methyl-2-trifluoromethyl-furan-3-carboxylic acid (1-methylcarbamoyl-2-phenyl-ethyl)-amide
Compound 3: 1-Methyl-3-trifluoromethyl-1H-pyrazole-4-carboxylic acid (1-methylcarbamoyl-2-phenyl-ethyl)-amide
Compound 4: Methyl-carbamic acid-2-methylcarbamoyl-2-[(1-methyl-3-trifluoromethyl-1H-pyrazole-4-carbonyl)-amino]-ethyl ester
Compound 5: Methyl-carbamic acid-2-methylcarbamoyl-2-[(1-methyl-3-trifluoromethyl-1H-pyrazole-4-carbonyl)-amino]-1-(tetrahydro-pyran-4-yl)-ethyl ester
Compound 6: Methyl-carbamic acid 1,1-dimethyl-2-methylcarbamoyl-2-[(1-methyl-3-trifluoromethyl-1H-pyrazole-4-carbonyl)-amino]-ethyl ester
Compound 7: 1-Methyl-3-trifluoromethyl-1H-pyrazole-4-carboxylic acid (2-hydroxy-3-methoxy-2-methoxymethyl-1-methylcarbamoyl-propyl)-amide
Compound 8: Methyl-carbamic acid 1,1-bis-methoxymethyl-2-methylcarbamoyl-2-[(1-methyl-3-trifluoromethyl-1H-pyrazole-4-carbonyl)-amino]-ethyl ester
Compound 9: Methyl-carbamic acid (E)-1-methyl-1-{methylcarbamoyl-[(1-methyl-3-trifluoromethyl-1H-pyrazole-4-carbonyl)-amino]-methyl}-but-2-enyl ester

The compounds were applied at two different dose rates (15 and 7.5 g ai/ha) and the spray volume was 750 I/ha. Six replicate pots with ten plants each were used per treatment. After 18 days plants were harvested and growth parameters were determined. The recorded parameters were fresh weight (FW) and dry weight (DW). Mean values of results, standard deviation (SD) and percentage to control were calculated. The results are listed in table 25.

TABLE 25
Effects on shoot fresh weight (FW) and dry weight (DW)

| | Shoot | ±SD | % of | Shoot | ±SD | % of |
|---|---|---|---|---|---|---|
| Treatments | FW [g] | [g] | control | DW [g] | [g] | control |
| Control | 30.6 | 2.2 | 100.0 | 2.8 | 0.2 | 100.0 |
| Compound 2 | 38.7 | 0.7 | 126.5 | 3.7 | 0.2 | 130.9 |
| 15 g/ha | | | | | | |
| Compound 2 | 40.9 | 1.2 | 133.7 | 3.8 | 0.1 | 135.2 |
| 7.5 g/ha | | | | | | |
| Compound 3 | 39.1 | 1.7 | 127.8 | 3.8 | 0.3 | 134.9 |
| 15 g/ha | | | | | | |
| Compound 3 | 39.4 | 2.4 | 128.8 | 3.8 | 0.2 | 134.7 |
| 7.5 g/ha | | | | | | |
| Compound 4 | 40.1 | 0.8 | 131.0 | 3.8 | 0.2 | 136.6 |
| 15 g/ha | | | | | | |
| Compound 4 | 37.7 | 1.7 | 123.2 | 3.7 | 0.2 | 130.9 |
| 7.5 g/ha | | | | | | |
| Compound 5 | 39.6 | 2.3 | 129.4 | 3.7 | 0.2 | 133.2 |
| 15 g/ha | | | | | | |
| Compound 5 | 41.2 | 1.9 | 134.6 | 3.8 | 0.1 | 135.4 |
| 7.5 g/ha | | | | | | |
| Compound 6 | 38.2 | 2.3 | 124.8 | 3.6 | 0.4 | 129.5 |
| 15 g/ha | | | | | | |
| Compound 6 | 39.2 | 2.7 | 128.1 | 3.7 | 0.3 | 132.1 |
| 7.5 g/ha | | | | | | |
| Compound 7 | 40.2 | 1.9 | 131.4 | 3.7 | 0.2 | 133.2 |
| 15 g/ha | | | | | | |
| Compound 7 | 38.7 | 1.8 | 126.5 | 3.7 | 0.2 | 131.4 |
| 7.5 g/ha | | | | | | |
| Compound 8 | 39.4 | 1.3 | 120.6 | 3.7 | 0.3 | 131.3 |
| 15 g/ha | | | | | | |
| Compound 8 | 39.6 | 1.7 | 129.4 | 3.9 | 0.1 | 137.8 |
| 7.5 g/ha | | | | | | |
| Compound 9 | 39.4 | 1.9 | 128.8 | 3.8 | 0.3 | 135.1 |
| 15 g/ha | | | | | | |
| Compound 9 | 38.8 | 2.1 | 126.8 | 3.6 | 0.3 | 129.1 |
| 7.5 g/ha | | | | | | |

As shown in table 25, all tested compounds (compounds 2 to 9) significantly enhanced the yield as an indicator of plant health by stimulating shoot growth. The increase in fresh- and dry weight was found to be in the range of 20 to 40% related to the untreated control plants.

1-15. (canceled)

16. A method for improving the health of a plant, which method comprises treating a seed from which the plant is grown, the plant and/or the locus where the plant is growing or is intended to grow and/or the plant propagules with a composition comprising a plant health improving amount of at least one compound (A) of formula I, 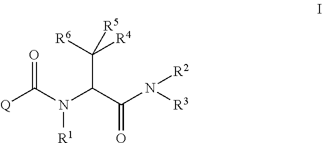
wherein:
Q is 5- or 6-membered heteroaryl having one to four nitrogen atoms, or having one to three nitrogen atoms and one oxygen or sulfur atom, or having one oxygen or sulfur atom, wherein the heteroaryl may be partially or fully halogenated and/or may carry 1 to 3 radicals selected from the group consisting of hydroxyl, cyano, C₁-C₆-alkyl, C₃-C₆-cycloalkyl, C₁-C₆-haloalkyl, C₁-C₆-alkoxy, C₁-C₆-haloalkoxy and C₁-C₆-alkoxy-C₁-C₄-alkyl;
R¹, R² are hydrogen or hydroxyl;
R³ is C₁-C₆-alkyl, C₁-C₄-cyanoalkyl or C₁-C₆-haloalkyl;
R⁴ is hydrogen, C₁-C₆-alkyl, haloalkyl or C₁-C₆-alkoxy-C₁-C₆-alkyl;
R⁵ is hydrogen, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁-C₆-cyanoalkyl, C₂-C₆-cyanoalkenyl, C₂-C₆-cyanoalkynyl, C₁-C₆-hydroxyalkyl, C₂-C₆-hydroxyalkenyl, C₂-C₆-hydroxyalkynyl, C₃-C₆-cycloalkyl, C₃-C₆-cycloalkenyl, 3- to 6-membered heterocyclyl,
wherein the cycloalkyl, cycloalkenyl or 3- to 6-membered heterocyclyl radicals mentioned above may be partially or fully halogenated and/or may carry one to three radicals selected from the group consisting of oxo, cyano, nitro, C₁-C₆-alkyl, C₁-C₆-haloalkyl, hydroxyl, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkoxycarbonyl-C₁-C₆-alkoxy, amino, C₁-C₆-alkylamino, di(C₁-C₆-alkyl)amino, C₁-C₆-alkylsulfonylamino, C₁-C₆-haloalkylsulfonylamino, aminocarbonylamino, (C₁-C₆-alkylamino)-carbonylamino, and di(C₁-C₆-alkyl)aminocarbonylamino,
C₁-C₆-alkoxy-C₁-C₄-alkyl, C₂-C₆-alkenyloxy-C₁-C₄-alkyl, C₂-C₆-alkynyloxy-C₁-C₄-alkyl, C₁-C₆-haloalkoxy-C₁-C₄-alkyl, C₂-C₆-haloalkenyloxy-C₁-C₄-alkyl, C₂-C₆-haloalkynyloxy-C₁-C₄-alkyl, C₁-C₆-alkoxy-C₁-C₄-alkoxy-C₁-C₄-alkyl, C₁-C₆-alkylthio-C₁-C₄-alkyl, C₂-C₆-alkenylthio-C₁-C₄-alkyl, C₂-C₆-alkynylthio-C₁-C₆-alkyl, C₁-C₆-haloalkyl-C -C₄-thioalkyl, C₂-C₆-haloalkenyl-C₁-C₄-thioalkyl, C₂-C₆-haloalkynyl-C₁-C₄-thioalkyl, C₁-C₆-alkylsulfinyl-C₁-C₄-alkyl, C₁-C₆-haloalkylsulfinyl-C₁-C₄-alkyl, C₁-C₆-alkylsulfonyl-C₁-C₄-alkyl, C₁-C₆-haloalkylsulfonyl-C₁-C₄-alkyl, amino-C₁-C₄-alkyl, C₁-C₆-alkylamino-C₁-C₄-alkyl, di(C₁-C₆-alkyl)amino-C₁-C₄-alkyl, C₁-C₆-alkylsulfonylamino-C₁-C₄-alkyl, C₁-C₆-alkylsulfonyl(C₁-C₆-alkyl)amino-C₁-C₄-alkyl, C₁-C₆-alkylcarbonyl, hydroxycarbonyl, C₁-C₆-alkoxycarbonyl, aminocarbonyl, C₁-C₆-alkylaminocarbonyl, di(C₁-C₆-alkyl)aminocarbonyl, formylamino-C₁-C₄-alkyl, C₁-C₆-alkoxycarbonylamino-C₁-C₄-alkyl, C₁-C₆-alkylcarbonyl-C₁-C₆-alkyl, hydroxycarbonyl-C₁-C₄-alkyl, C₁-C₆-alkoxycarbonyl-C₁-C₆-alkyl, C₁-C₆-haloalkoxycarbonyl-C₁-C₄-alkyl, C₁-C₆-alkylcarbonyloxy-C₁-C₄-alkyl, aminocarbonyl-C₁-C₄-alkyl, C₁-C₆-alkylaminocarbonyl-C₁-C₄-alkyl, di(C₁-C₆-alkyl)aminocarbonyl-C₁-C₄-alkyl, C₁-C₆-alkylcarbonylamino-C₁-C₄-alkyl, C₁-C₆-alkylcarbonyl-(C₁-C₆-alkylamino)-C₁-C₄-alkyl, (C₁-C₆-alkyl)aminocarbonyloxy-C₁-C₄-alkyl, di(C₁-C₆-alkyl)aminocarbonyloxy-C₁-C₄-alkyl, [(C₁-C₆-alkyl)aminocarbonylamino]C₁-C₄-alkyl, [di(C₁-C₆-alkyl)aminocarbonylamino]C₁-C₄-alkyl;
phenyl, phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl, phenyl-C₂-C₄-haloalkynyl, phenyl-C₁-C₄-hydroxyalkyl, phenyl-C₂-C₄-hydroxyalkenyl, phenyl-C₂-C₄-hydroxyalkynyl, phenylcarbonyl-C₁-C₄-alkyl, phenylcarbonyloxy-C₁-C₄-alkyl, phenyloxycarbonyl-C₁-C₄-alkyl, phenyloxy-C₁-C₄-alkyl, phenylthio-C₁-C₄-alkyl, phenylsulfinyl-C₁-C₄-alkyl, phenylsulfonyl-C₁-C₄-alkyl, heteroaryl, heteroaryl-C₁-C₄-alkyl, heteroaryl-C₂-C₄-alkenyl, heteroaryl-C₂-C₄-alkynyl, heteroaryl-C₁-C₄-haloalkyl, heteroaryl-C₂-C₄-haloalkenyl, heteroaryl-C₂-C₄-haloalkynyl, heteroaryl-C₁-C₄-hydroxyalkyl, heteroaryl-C₂-C₄-hydroxyalkenyl, heteroaryl-C₂-C₄-hydroxyalkynyl, heteroarylcarbonyl-C₁-C₄-alkyl, heteroarylcarbonyloxy-C₁-C₄-alkyl, heteroaryloxycarbonyl-C₁-C₄-alkyl, heteroaryloxy-C₁-C₄-alkyl, heteroarylthio-C₁-C₄-alkyl, heteroarylsulfinyl-C₁-C₄-alkyl, or heteroarylsulfonyl-C₁-C₄-alkyl,
wherein the phenyl and heteroaryl radicals mentioned above may be partially or fully halogenated and/or may carry one to three radicals selected from the group consisting of cyano, nitro, C₁-C₆-alkyl, C₁-C₆-haloalkyl, hydroxyl, C₁-C₆-hydroxyalkyl, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkoxycarbonyl-C₁-C₆-alkoxy, amino, C₁-C₆-alkylamino, di(C₁-C₆-alkyl)amino, C₁-C₆-alkylsulfonyl , C₁-C₆-alkylsulfinyl, C₁-C₆-alkylthio, C₁-C₆-alkylsulfonylamino, C₁-C₆-haloalkylsulfonylamino, (C₁-C₆-alkylamino)-carbonylamino, di(C₁-C₆-alkyl)-aminocarbonylamino, aryl and aryl(C₁-C₆-alkyl); or
R⁴ and R⁵ form together a 3-10-membered ring which may contain one to four nitrogen atoms, one to three nitrogen atoms and one oxygen or sulfur atom, one oxygen or sulfur atom, or 2 oxygen atoms whereas the ring may be partially or fully halogenated and/or may carry 1 to 3 radicals selected from the group consisting of cyano, C₁-C₆-alkyl, C₃-C₆-cycloalkyl, C₁-C₆-haloalkyl, C₁-C₆-alkoxy C₁-C₆-haloalkoxy and C₁-C₆-alkoxy-C₁-C₄-alkyl;
R⁶ is hydrogen, C₁-C₆-alkyl or OR⁷ or NR⁸R⁹
R⁷ is hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, formyl, C₁-C₆-alkylcarbonyl, C₃-C₆-cycloalkylcarbonyl, C₂-C₆-alkenylcarbonyl, C₂-C₆-alkynylcarbonyl, C₁-C₆-alkoxycarbonyl, C₃-C₆-alkenyloxycarbonyl, C₃-C₆-alkynyloxycarbonyl, aminocarbonyl, C₁-C₆-alkylaminocarbonyl, C₃-C₆-alkenylaminocarbonyl, C₃-C₆-alkynylaminocarbonyl, C₁-C₆-alkylsulfonylaminocarbonyl, di(C₁-C₆-alkyl)aminocarbonyl, N—(C₃-C₆-alkenyl)-N—(C₁-C₆-alkyl)aminocarbonyl, N—(C₃-C₆-alkynyl)-N—(C₁-C₆-alkyl)aminocarbonyl, N—(C₁-C₆-alkoxy)-N—(C₁-C₆-alkyl)aminocarbonyl, N—(C₃-C₆-alkenyl)-N—(C₁-C₆-alkoxy)aminocarbonyl, N—(C₃-C₆-alkynyl)-N—(C₁-C₆-alkoxy)aminocarbonyl, C¹-C⁶-alkylamino-thiocarbonyl, di(C₁-C₆-alkyl)aminothiocarbonyl, (C₁-C₆-alkyl)cyanoimino, (amino)cyanoimino, [(C₁-C₆-alkyl)amino]cyanoimino, di(C₁-C₆-alkyl)aminocyanoimino, C₁-C₆-alkylcarbonyl-C₁-C₆-alkyl, C₁-C₆-alkoxyimino-C₁-C₆-alkyl, N—(C₁-C₆-alkylamino)imino-C₁-C₆-alkyl, N-[di(C₁-C₆-alkyl)amino]imino-C₁-C₆-alkyl, tri-C₁-C₄-alkylsilyl,
wherein the alkyl, cycloalkyl and alkoxy radicals mentioned above may be partially or fully halogenated and/or may carry one to three groups selected from the group consisting of cyano, hydroxyl, C₃-C₆-cycloalkyl, C₁-C₆-alkoxy-C₁-C₄-alkyl, C₁-C₄-alkoxy-C₁-C₄-alkoxy-C₁-C₄-alkyl, C₁-C₄-alkoxy, C₁-C₄-alkylthio, di(C₁-C₄-alkyl)amino, C₁-C₄-alkyl-C₁-C₆-alkoxycarbonylamino, C₁-C₄-alkylcarbonyl, hydroxycarbonyl, C₁-C₄-alkoxycarbonyl, aminocarbonyl, C₁-C₄-alkylaminocarbonyl, di(C₁-C₄-alkyl)aminocarbonyl and C₁-C₄-alkylcarbonyloxy;
phenyl, phenyl-C₁-C₆-alkyl, phenylcarbonyl-C₁-C₆-alkyl, phenoxycarbonyl, phenylaminocarbonyl, phenylsulfonylaminocarbonyl, N—(C₁-C₆-alkyl)-N-(phenyl)aminocarbonyl, phenyl-C₁-C₆-alkylcarbonyl, phenylcarbonyl, wherein the phenyl radical may be partially or fully halogenated and/or may carry one to three groups selected from the group consisting of nitro, cyano, C₁-C₄-haloalkyl, C₁-C₄-alkoxy and C₁-C₄-haloalkoxy;
R⁸ is hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, formyl, C₁-C₆-alkylcarbonyl, C₃-C₆-cycloalkylcarbonyl, C₂-C₆-alkenylcarbonyl, C₂-C₆-alkynylcarbonyl, C₁-C₆-alkoxycarbonyl, C₃-C₆-alkenyloxycarbonyl, C₃-C₆-alkynyloxycarbonyl, aminocarbonyl, C₁-C₆-alkylaminocarbonyl, C₃-C₆-alkenylaminocarbonyl, C₃-C₆-alkynylaminocarbonyl, C₁-C₆-alkylsulfonylaminocarbonyl, di(C₁-C₆-alkyl)aminocarbonyl, N—(C₃-C₆-alkenyl)-N—(C₁-C₆-alkyl)aminocarbonyl, N—(C₃-C₆-alkynyl)-N—(C₁-C₆-alkyl)aminocarbonyl, N—(C₁-C₆-alkoxy)-N—(C₁-C₆-alkyl)aminocarbonyl, N—(C₃-C₆-alkenyl)-N—(C₁-C₆-alkoxy)aminocarbonyl, N—(C₃-C₆-alkynyl)-N—(C₁-C₆-alkoxy)aminocarbonyl, di(C₁-C₆-alkyl)aminothiocarbonyl, (C₁-C₆-alkyl)cyanoimino, (amino)cyanoimino, [(C₁-C₆-alkyl)amino]cyanoimino, di(C₁-C₆-alkyl)aminocyanoimino, C₁-C₆-alkylcarbonyl-C₁-C₆-alkyl, C₁-C₆-alkoxyimino-C₁-C₆-alkyl, N—(C₁-C₆-alkylamino)imino-C₁-C₆-alkyl, N-[di(C₁-C₆-alkyl)amino]imino-C₁-C₆-alkyl
wherein the alkyl, cycloalkyl and alkoxy radicals mentioned may be partially or fully halogenated and/or may carry one to three groups selected from the group consisting of cyano, hydroxyl, C₃-C₆-cycloalkyl, C₁-C₆-alkoxy-C₁-C₄-alkoxy-C₁-C₄-alkoxy-C₁-C₄-alkyl, C₁-C₄-alkoxy, C₁-C₄-alkylthio, di(C₁-C₄-alkyl)amino, C₁-C₄-alkyl-C₁-C₆-alkoxycarbonylamino, C₁-C₄-alkylcarbonyl, hydroxycarbonyl, C₁-C₄-alkoxycarbonyl, aminocarbonyl, C₁-C₄-alkylaminocarbonyl, di(C₁-C₄-alkyl)aminocarbonyl and C₁-C₄-alkylcarbonyloxy;
phenyl, phenyl-C₁-C₆-alkyl, phenylcarbonyl-C₁-C₆-alkyl, phenoxycarbonyl, phenylaminocarbonyl, phenylsulfonylaminocarbonyl, N—(C₁-C₆-alkyl)-N-(phenyl)aminocarbonyl, phenyl-C₁-C₆-alkylcarbonyl, phenylcarbonyl wherein the phenyl radical may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy;
R⁹ is hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, hydroxyl or C₁-C₆-alkoxy;
or an agriculturally useful salt thereof.

17. The method according to claim 16, wherein the compound (A) of formula I is selected from the group of heteroaroyl-substituted alanines consisting of
Dimethyl-carbamic acid 2-methylcarbamoyl-2-[(1-methyl-3-trifluoromethyl-1H-pyrazole-4-carbonyl)-amino]-1-(tetrahydro-pyran-4-yl)-ethyl ester,
5-Methyl-2-trifluoromethyl-furan-3-carboxylic acid (1-methylcarbamoyl-2-phenyl-ethyl)-amide,
1-Methyl-3-trifluoromethyl-1H-pyrazole-4-carboxylic acid (1-methylcarbamoyl-2-phenyl-ethyl)-amide,
Methyl-carbamic acid-2-methylcarbamoyl-2-[(1-methyl-3-trifluoromethyl-1H-pyrazole-4-carbonyl)-amino]-ethyl ester,
Methyl-carbamic acid 2-methylcarbamoyl-2-[(1-methyl-3-trifluoromethyl-1H-pyrazole-4-carbonyl)-amino]-1-(tetrahydro-pyran-4-yl)-ethyl ester,
Methyl-carbamic acid 1,1-dimethyl-2-methylcarbamoyl-2-[(1-methyl-3-trifluoromethyl-1H-pyrazole-4-carbonyl)-amino]-ethyl ester,
1-Methyl-3-trifluoromethyl-1H-pyrazole-4-carboxylic acid (2-hydroxy-3-methoxy-2-methoxymethyl-1-methylcarbamoyl-propyl)-amide,
Methyl-carbamic acid 1,1-bis-methoxymethyl-2-methylcarbamoyl-2-[(1-methyl-3-trifluoromethyl-1H-pyrazole-4-carbonyl)-amino]-ethyl ester,
Methyl-carbamic acid (E)-1-methyl-1-{methylcarbamoyl-[(1-methyl-3-trifluoromethyl-1H-pyrazole-4-carbonyl)-amino]-methyl}-but-2-enyl ester,
2-Methyl-5-trifluoromethyl-oxazole-4-carboxylic acid (1-methylcarbamoyl-2-phenyl-ethyl)-amide,
2-Trifluoromethyl-thiophene-3-carboxylic acid (1-methylcarbamoyl-2-phenyl-ethyl)-amide,
Methyl-carbamic acid 1-methyl-2-methylcarbamoyl-2-[(5-methyl-2-trifluoromethyl-furan-3-carbonyl)-amino]-ethyl ester,
Methyl-carbamic acid 1-methyl-2-methylcarbamoyl-2-[(1-methyl-3-trifluoromethyl-1H-pyrazole-4-carbonyl)-amino]-ethyl ester and
Methyl-carbamic acid 1,1-dimethyl-2-methylcarbamoyl-2-[(5-methyl-2-trifluoromethyl-furan-3-carbonyl)-amino]-ethyl ester.

18. The method according to claim 16, wherein the yield of a plant is increased.

19. The method according to claim 16, wherein the number of tillers is increased.

20. The method according to claim 16, wherein the shoot and/or root growth is increased.

21. The method according to claim 16, wherein at least one compound (A) of the formula I or an agriculturally useful salt thereof is applied to the plant and/or the locus where the plant is growing or is intended to grow in an amount of from 0.001 g to 4 kg a.i./ha.

22. The method according to claim 16, wherein at least one compound (A) of the formula I or an agriculturally useful salt thereof is applied to plant propagules.

23. The method according to claim 16, wherein the composition further comprises at least one agriculturally active compound (B) or an agriculturally useful salt thereof.

24. The method of claim 23, wherein the at least one agriculturally active compound (B) or an agriculturally useful salt thereof is selected from the group consisting of N-(3′,4′,5′-trifluorobiphenyl-2-yl)-1-methyl-3-difluoromethyl-1H-pyrazole-4-carboxamide, azoxystrobin, dimoxystrobin, fluoxastrobin, kresoxim-methyl, orysastrobin, picoxystrobin, pyraclostrobin, trifloxystrobin, bixafen, boscalid, isopyrazam, isotianil, metalaxyl, metalaxyl-M (mefenoxam), penthiopyrad, dimetomorph, zoxamide, mandipropamid, silthiofarm, cyproconazol, difenoconazole, epoxiconazole, fluquinconazole, flusilazole, flutriafol, metconazole, propiconazole, prothioconazole, tebuconazole, triticonazole, prochloraz, cyprodinil, pyrimethanil, fludioxonil, fenpropimorph, fenpropidin, iprodione, famoxadone, probenazole, proquinazid, quinoxifen, acibenzolar-S-methyl, metiram, thiram, guazatine, dithianon, fosetyl-aluminium, chlorothalonil, phthalide, thiophanate-methyl, cymoxanil, metrafenone, spiroxamine, chlormequat (chlormequat chloride), cyclanilide, ethephon, mepiquat (mepiquat chloride), N-6-benzyladenine, prohexadione (prohexadione-calcium), trinexapac-ethyl, 1-naphtaleneacetamide, dimethachlor, dimethenamid, flufenacet, mefenacet, metazachlor; glyphosate, glufosinate, fenoxaprop, fluazifop, propaquizafop, quizalofop-P-tefuryl, desmedipham, phenmedipham, prosulfocarb, clethodim, cycloxidim, sethoxidim, tepraloxidim, tralkoxydim, pendimethalin, acifluorfen, bifenox, bromoxynil, ioxynil, imazamethabenz, imazamox, imazapic, imazapyr, imazaquin, imazethapyr, 2,4 D, dichlorprop, MCPA, mecoprop, chloridazon, clopyralid, diflufenican, fluroxypyr, picloram, picolinafen, amidosulfuron, flupyrsulfuron, foramsulfuron, iodosulfuron, metsulfuron-methyl, nicosulfuron, prosulfuron, rimsulfuron, sulfosulfuron, thifensulfuron, triasulfuron, tribenuron, tritosulfuron, atrazine, metamitron, metribuzin, simazine, terbuthylazine, chlortoluron, isoproturon, florasulam, bentazone, clomazone, dicamba, diflufenzopyr, ethofumesate, isoxaflutole, propyzamide, quinclorac, quinmerac, mesotrione, sulcotrione, topremazone, triclopyr, pyridat, thiencarbazone, tefluryltrione, dimethoate, methiocarb, pirimicarb, bifenthrin, cyfluthrin, cyhalothrin, lambda-cyhalothrin, cypermethrin, alpha-cypermetrin, beta-cypermethrin, zeta-cypermethrin, deltamethrin, esfenvalerate, tefluthrin, clothianidin, imidacloprid, thiamethoxam, acetamiprid, thiacloprid, ethiprole, fipronil, metaflumizone, flonicamid, pymetrozine, chlorantriliprole, derivatives of vinylglycine, hydroxylamines, oxime ether derivatives, n-propyl gallate, polyamines such as putrescine, spermine, spermidine, salicylic acid including its synthetic analogon acibenzolar-S-methyl, and also triazolyl compounds, cyclopropene and its derivatives, 1-methylcyclopropene (1-MCP), 2,5-norbornadiene, 3-amino-1,2,4-triazole and Ag+ ions.

25. The method according to claim 16, wherein the plant is selected from agricultural, silvicultural and horticultural plants, each in natural or genetically modified form.

26. The method according to claim 16, wherein the plant is selected from the group consisting of alfalfa, annual grass, bamboo, barley, cacao, canola, castor-oil plant, coconut, coffee, cotton, eggplant, eucalyptus, flax, forage crops, grape, Jatropha, linseed, maize, manihot, manioc/cassava, Miscanthus species, oat, oil palm, Panicum species, pea, peanut, perennial grass, pine, poplar/cottonwood, potato, rapeseed, rice, rye, safflower, Salix species, silver maple, soybean, spruce, sugar beet, sugarcane, sunflower, sweet potato, tea, tobacco, tomato, triticale, Vicia species, grapes and wheat.

27. A fruit produced according to the method of claim 16.

28. A seed treated according to the method of claim 16.
